SEQUENCE LISTING (14 sequences)

SEQ ID NO: 1 - DNA, 22 bp, Artificial Sequence
  source: /note=“Description of Artificial Sequence Synthetic oligonucleotide”
gtgcactttgagctcgaatgag

SEQ ID NO: 2 - protein, 6 aa, Artificial Sequence
  source: /note=“Description of Artificial Sequence Synthetic peptide”
VHFELE

SEQ ID NO: 3 - protein, 587 aa, Artificial Sequence
  source: /note=“Description of Artificial Sequence Synthetic polypeptide”
MVTAAMLLQCCPVLARGPTSLLGKVVKTHQFLFGIGRCPILATQGPNCSQIHLKATKAGGDSPSWAKGHCPFMLSELQDGKSKIVQKAAPEVQEDVKAFKTDLPSSLVSVSLRKPFSGPQEQEQISGKVTHLIQNNMPGNYVFSYDQFFRDKIMEKKQDHTYRVFKTVNRWADAYPFAQHFSEASVASKDVSVWCSNDYLGMSRHPQVLQATQETLQRHGAGAGGTRNISGTSKFHVELEQELAELHQKDSALLFSSCFVANDSTLFTLAKILPGCEIYSDAGNHASMIQGIRNSGAAKFVFRHNDPDHLKKLLEKSNPKIPKIVAFETVHSMDGAICPLEELCDVSHQYGALTFVDEVHAVGLYGSRGAGIGERDGIMHKIDIISGTLGKAFGCVGGYIASTRDLVDMVRSYAAGFIFTTSLPPMVLSGALESVRLLKGEEGQALRRAHQRNVKHMRQLLMDRGLPVIPCPSHIIPIRVGNAALNSKLCDLLLSKHGIYVQAINYPTVPRGEELLRLAPSPHHSPQMMEDFVEKLLLAWTAVGLPLQDVSVAACNFCRRPVHFELMSEWERSYFGNMGPQYVTTYA

SEQ ID NO: 4 - DNA, 20 bp, Artificial Sequence
  source: /note=“Description of Artificial Sequence Synthetic oligonucleotide”
aaaagcaccgactcggtgcc

SEQ ID NO: 5 - DNA, 180 bp, Artificial Sequence
  source: /note=“Description of Artificial Sequence Synthetic polynucleotide”
ctggcctggactgaggtggggctgcccctccaagatgtgtctgtggctgcatgcaacttctgtcatcgtcctgtgcactttgagctcgaatgagtgggagcgatcctactttgggaacatgggaccccaatatgttaccacctatgcttaaggagccagctgccttggatgccagctcca

SEQ ID NO: 6 - RNA, 22 nt, Artificial Sequence
  source: /note=“Description of Artificial Sequence Synthetic oligonucleotide”
ugaaaaauuggucauaaccgaa

SEQ ID NO: 7 - protein, 16 aa, Artificial Sequence
  source: /note=“Description of Artificial Sequence Synthetic peptide”
DPDHLKKLLEKSNPKI

SEQ ID NO: 8 - DNA, 797 bp, Artificial Sequence
  source: /note=“Description of Artificial Sequence Synthetic polynucleotide”
gtgaggctgggcacagtagctcatacctgtaatcccagcactttgggaggctgaagcgggagaagatcacttgaggccaggagtttgaggccagccttggcaacatagcaagacactatctctaccaaaacagtttttttaattagccaggcatgatggcttgcatctatagtcccagctactcaggaggctaaggtaggaagattgcttaagcccaagacttcaaggttcagtgagctatgatcacgctattgcactccagcctgaatgacagacagagactcagtttctaggaaaaacaaaaaaatgtatggtgagttgaggcttgaaagccatatcccttgcttgcgtgagcaggtgcttttggttgtggtgactacaggtgctgttggtagcctgccttctgtcctttaatacttaccgtctacacatggcactgagccaaacacgacatacagtcttatttacttacaagatgaggacatcaaataacctgcccaggatctccctcagttgccaagctgagacttgttgtatttcaccaaaatgcagctgtgtttcacaaccaccattctgtactgtcttttgttcaatttttagctgtttccacttttcagtatgatcgtttctttgagaaaaaaattgatgagaaaaagaatgaccacacctatcgagtttttaaaactgtgaaccggcgagcacacatcttccccatggcagatgactattcagactccctcatcaccaaaaagcaagtgtcagtctggtgcagtaatgactacctaggaatgagtcgc

SEQ ID NO: 9 - DNA, 794 bp, Artificial Sequence
  source: /note=“Description of Artificial Sequence Synthetic polynucleotide”
tgtggggcagttatgtaagtagcccttggctttcaaatattactgttgttatttggcaagccaatgatgatgtataggggttggatcttttatggagggaacattcagtagctgaaagtgtgccatagcaaaatactattcttagcttctgaaaaatatctacagattacttttaaaggaactctaatatgcaggtagctgctggagccccttaacttgtgagggttcaagcttataggctcaatgaccacacacgtcagtccactttctatcactctgcaagtgagtgtgcctttaacaccaggcagtctttcacagagacatccttggttgtggctgtgagtggagaaatacgaatctagcagcgctagagaaaaagctactccaaaagacatgttaaaaaggatggtagatcccatcactcgtccttcaggaggctcaccatcctagggatgctggagagagttgctggccagattctccaaaggcttgctaaagagggtcccggtttgaggtttaatgatagtgatatatccaaacattacatgactggcttgcagagggtaaccaccatccaggatgtccctctagagtttgctttttcttaagctaacatgttattggaaagaataatgtttctcagaaaataaaccttagtttcttagaaaagaaactcagctaataccaagcacttactgactgttaaatgaacacttgttcttgatgattcctggaggtatcatcaccctagcattgacaccttctcccacctagtctgaagatgaaatcagttg

SEQ ID NO: 10 - DNA, 20 bp, Artificial Sequence
  source: /note=“Description of Artificial Sequence Synthetic oligonucleotide”
gatggcacacagcttccgtc

SEQ ID NO: 11 - DNA, 21 bp, Artificial Sequence
  source: /note=“Description of Artificial Sequence Synthetic oligonucleotide”
gaccatattgaaacgctgtat

SEQ ID NO: 12 - DNA, 40 bp, Artificial Sequence
  source: /note=“Description of Artificial Sequence Synthetic oligonucleotide”
ttaatacgactcactataggctttgaacttatgagcgagt

SEQ ID NO: 13 - DNA, 24 bp, Artificial Sequence
  source: /note=“Description of Artificial Sequence Synthetic oligonucleotide”
gtgcactttgaacttatgagcgag

SEQ ID NO: 14 - protein, 8 aa, Artificial Sequence
  source: /note=“Description of Artificial Sequence Synthetic peptide”
VHFELMSE

(19) United States
(12) Patent Application Publication
(10) Pub. No.: US 2024/0276957 A1
(43) Pub. Date: Aug. 22, 2024

(54) METHODS OF TREATMENT WITH AMINOLEVULINIC ACID SYNTHASE 2 (ALAS2) MODULATORS

(71) Applicant: Agios Pharmaceuticals, Inc., Cambridge, MA (US)

(72) Inventors: Giovanni Cianchetta, Boxford, MA (US); Peng Liu, Belmont, MA (US); Shengfang Jin, Newton, MA (US); Brian Christopher Shook, Cambridge, MA (US); Zhihua Sui, Audubon, PA (US); Kai Lu, Irvine, CA (US); Barden Chan, Brookline, MA (US); Gavin Whissell, Cambridge, MA (US)

(21) Appl. No.: 17/616,790

(22) PCT Filed: Jun. 5, 2020
(86) PCT No.: PCT/US2020/036424

§ 371 Date: Dec. 6, 2021

Related U.S. Application Data
(60) Provisional application No. 62/893,942, filed on Aug. 30, 2019.
(60) Provisional application No. 62/858,699, filed on Jun. 7, 2019.

(51) Int. Cl. A01K 67/0275; C12N 9/10; C12N 9/22; C12N 15/11; C12N 15/85

(52) U.S. Cl. CPC A01K 67/0275; C12N 9/1029; C12N 9/22; C12N 15/11; C12N 15/8509; C12Y 203/01037; A01K 2217/054; A01K 2217/075; A01K 2227/105; A01K 2267/0306; C12N 2015/8536; C12N 2310/20; C12N 2800/80

(57) ABSTRACT

Described herein is a compound of Formula I or a pharmaceutically acceptable salt thereof: wherein Ring A R¹, R², a, b, and n are as defined herein. Also described is a method of treating a subject having a disorder in need of treatment, comprising inhibiting aminolevulinic acid synthase 2 (ALAS2) in the subject by administering a compound of Formula (I) or a pharmaceutically acceptable salt thereof. Disorders that are of particular interest are blood disorders, such as porphyria and anemia.

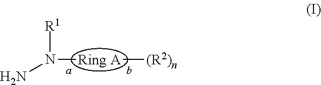

### REFERENCE TO RELATED APPLICATIONS

This application claims the benefit of the filing dates of U.S. Provisional Patent Application Nos. 62/858,699, filed on Jun. 7, 2019, and 62/893,942, filed on Aug. 30, 2019, the entire contents of each of which are incorporated herein by reference.

### BACKGROUND

Heme is an essential molecule to almost all organisms. Heme functions as a prosthetic group on several types of proteins, including cytochromes, catalases, hemoglobin, and myoglobin. Moreover, it has been reported that heme is also involved in numerous regulatory systems in mammals (J. Bio. Chem., 2016, 20516). Production of heme and heme precursors are regulated by 5-aminolevulinatye synthases (e.g. ALAS2). Inhibition of ALAS2 or reduction in ALAS2 protein level may reduce heme pathway flux and suppress the production of toxin metabolites in the heme pathway.

### SUMMARY

Inhibition of ALAS2 can reduce levels of metabolites of the heme biosynthesis pathway in animal models (see Example 36). One aspect of the present invention provides compounds of Formula (I) as described herein as inhibitors of ALAS2 (see Example 38). Based on these results, novel ALAS2 inhibitors and methods of treating disorders characterized by accumulation of metabolites of the heme pathway are disclosed.

The present disclosure is directed to a compound of Formula (I) or a pharmaceutically acceptable salt thereof:

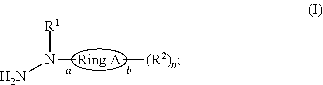

wherein:
Ring A is a 5-membered heteroaryl;
R¹ is hydrogen, C₁-C₆ alkyl, C₁-C₆ haloalkyl, —CO₂R³, -C₁-C₆ acyl, or C₃-C₆ cycloalkyl; wherein each of C₁-C₆ alkyl, C₁-C₆ haloalkyl, —C₁-C₆ acyl, and C₃-C₆ cycloalkyl is independently substituted with 0-3 instances of halogen or hydroxyl;
R² is hydrogen, —CN, hydroxyl, C₁-C₆ alkyl, C₃-C₈ cycloalkyl, 3-14 membered heterocyclyl, 8-12 membered bicyclic heteroaryl, 5- or 6-membered monocyclic heteroaryl, phenyl, or naphthyl; wherein each of C₁-C₆ alkyl, C₃-C₈ cycloalkyl, 3-14 membered heterocyclyl, 8-12 membered bicyclic heteroaryl, 5- or 6-membered monocyclic heteroaryl, phenyl, and naphthyl is independently substituted with 0-4 instances halogen, hydroxyl, C₁-C₆ alkyl, C₁-C₆ alkoxy, phenyl, N(R^{a})₂, or —CO₂R^{b}; each of R³, R^{a} and R^{b} is independently C₁-C₆ alkyl;
n is 0, 1, 2, 3, 4, or 5; and
a and b each indicate points of attachment of Ring A.

Another aspect of the invention provides methods of treating a subject in need of treatment of a disorder treatable by inhibiting aminolevulinic acid synthase (ALAS2), the method comprising administering to the subject a compound of Formula (I) or a pharmaceutically acceptable salt thereof.

A further aspect of the invention provides a mouse model of human XLP (X-linked dominant protoporphyria), which may be useful, among others, for testing efficacy of candidate compounds and effective doses thereof for treating XLP.

### BRIEF DESCRIPTION OF THE DRAWINGS

FIG. 1A and B depict that ALAS2 is the rate limiting step in PPIX accumulation. Mass isotope distribution was used to empirically determine ALAS2 was the rate limiting step in PPIX accumulation in an EPP cell model using ¹³C₂-glycine (See Example 36.1). PPIX is a polymer of ALA (product of ALAS2). One PPIX molecule is derived from 8 molecules of ALA. Thus, when ¹³C₂-glycine was introduced to the cells, the pool of PPIX generated would be composed of molecules containing different number of ¹³C carbon, ranging from 0 to 8. At earlier time points, since the % of ¹³C-ALA was low, the isotopomer distribution trended to have PPIX molecules carrying lower number of labeled carbons. % ¹³C-ALA increased as labeling time increased, leading to a shift in the isotopomer distribution to have PPIX molecules with more labeled carbons. Since PPIX isotopomer distribution was mathematically defined by % ¹³C-ALA, by directly measuring ¹³C-labeled PPIXs and their mass distribution, one could predict what the % ALA should have been at each time point. At the same time, the actual % ALA in cells at each time point was also measured directly by LC-MS. As shown in the diagram on the right, these two curves were statistically identical, indicating there was no slow step between ALA and PPIX and thus demonstrating ALAS2 was the rate limiting step in the production of PPIX in EPP cells.

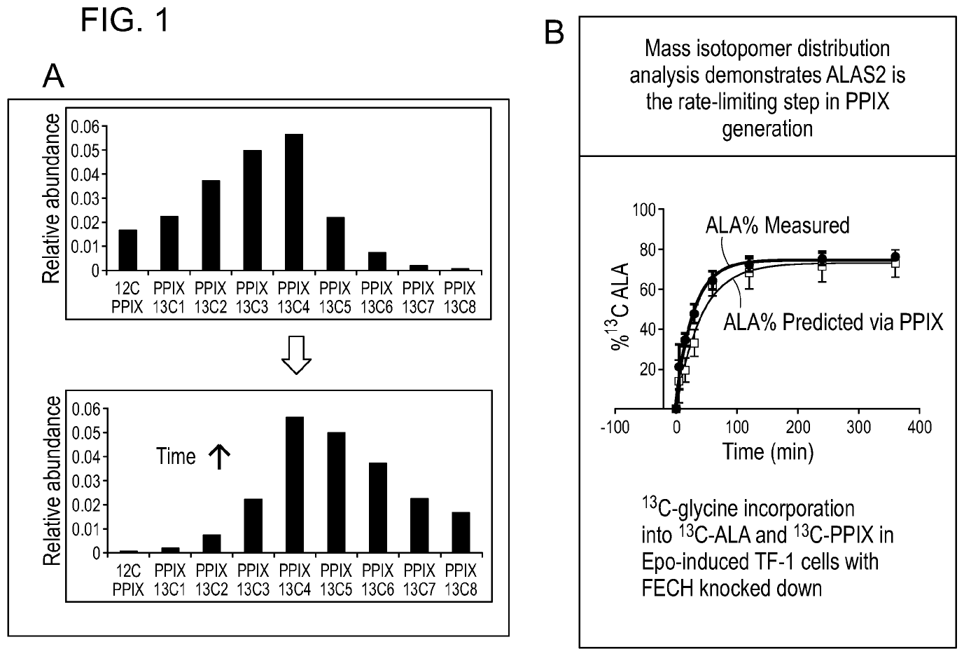

FIG. 2 depicts the mouse models for ALAS2 target validation to treat erythroid porphyria. XLP mouse model with ALAS2-delAT mutation recapitulates human disease markers. To create an XLP mouse model, an ALAS2 C-terminal deletion (SEQ ID NO:1 and SEQ ID NO:2) was provided in mice similar to that in human XLP disease by knocking in nucleotide deletions and mutation via CRISPR/CAS9 (Example 36.2). Indeed, these mutations resulted in a shorter form of the protein compared to full-length ALAS2 as visualized by western blot. Significantly, these mice showed elevated level of blood PPIX and Zn-PPIX, two characteristic disease markers seen in human XLP patients.

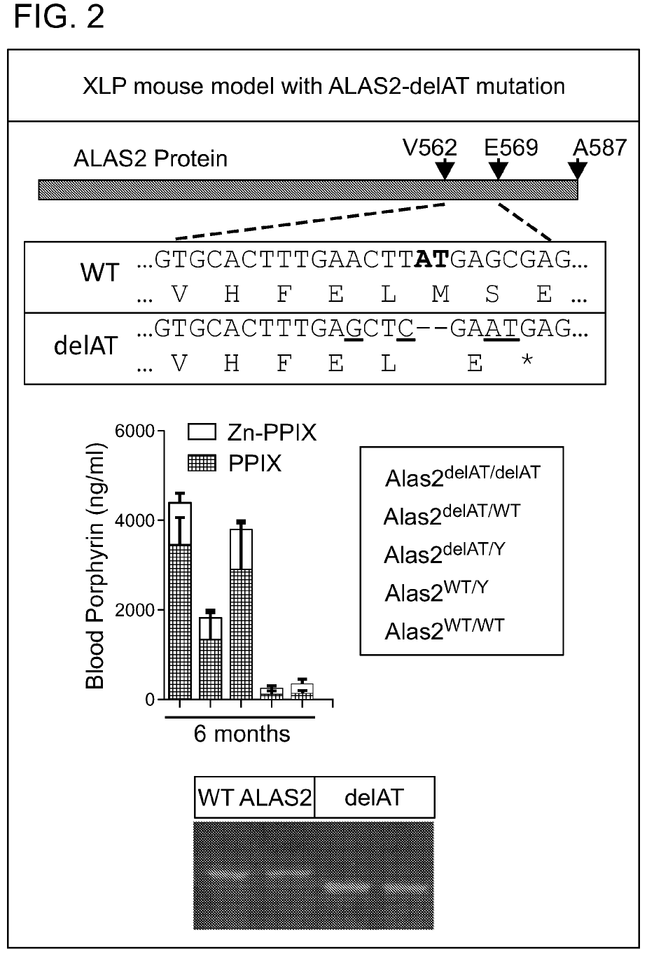

FIG. 3 depicts mouse models for ALAS2 target validation to treat erythroid porphyria. ALAS2 protein level could be knocked down by shRNA in mice. The expression cassettes for rtTA and a shRNA against mouse ALAS2 were introduced into C₅₇B6 (Example 36.3). Homozygous rtTa^{+/+}/ShALAS2^{+/+} mice were tested and showed that ALAS2 could be significantly knocked down upon doxycycline treatment for as short as 4 days. Heterozygous rtTA^{+/+}/shALAS2^{+/-} mice were tested to confirm if doxycycline treatment of these mice would have lesser extent of protein knockdown as these mice only carried one copy of the shRNA expression cassette. Indeed, quantification of ALAS2 protein blot showed that about 50% of ALAS2 was knocked down in heterozygous rtTA^{+/-}/shALAS2^{+/-} mice fed with doxycycline-containing chow for 16 days.

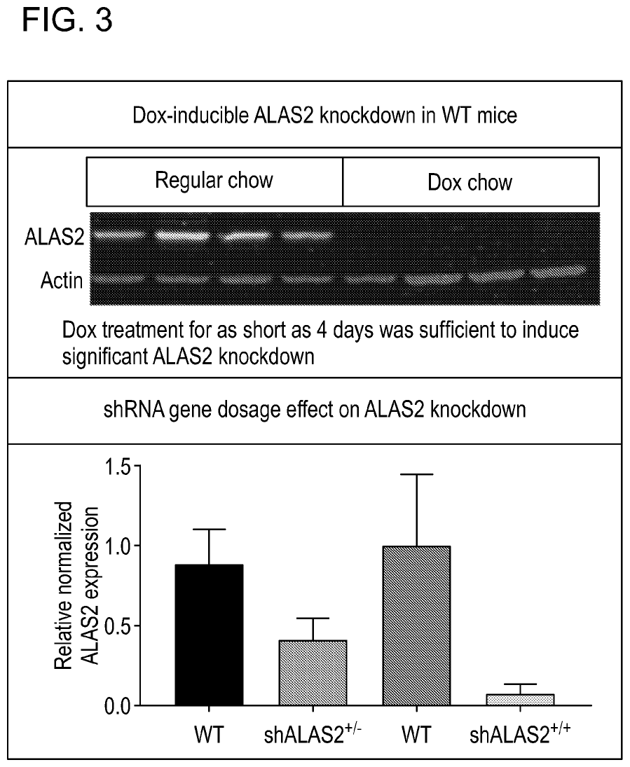

FIG. 4 depicts genetic validation of ALAS2 inhibition strategy. The delAT mice carrying one copy of an ALAS2 shRNA cassette was tested to validate ALAS2 inhibition strategy in treating XLP (Example 36.4). These mice were fed with doxycycline to induce the expression of an shRNA against ALAS2. Consistent with previous results (see FIG. 3), mice with one copy of the shALAS2 gene, upon doxycycline treatment, had ALAS2-delAT protein level reduced by about 50%. This reduction in ALAS2-delAT protein level was sufficient to reduce ALA in the bone marrow to WT level. Significantly, blood PPIX level was normalized to a level statistically indistinguishable from WT mice. Thus, the data indicated that inhibition of ALAS2 is a valid strategy to treat erythroporphyrias.

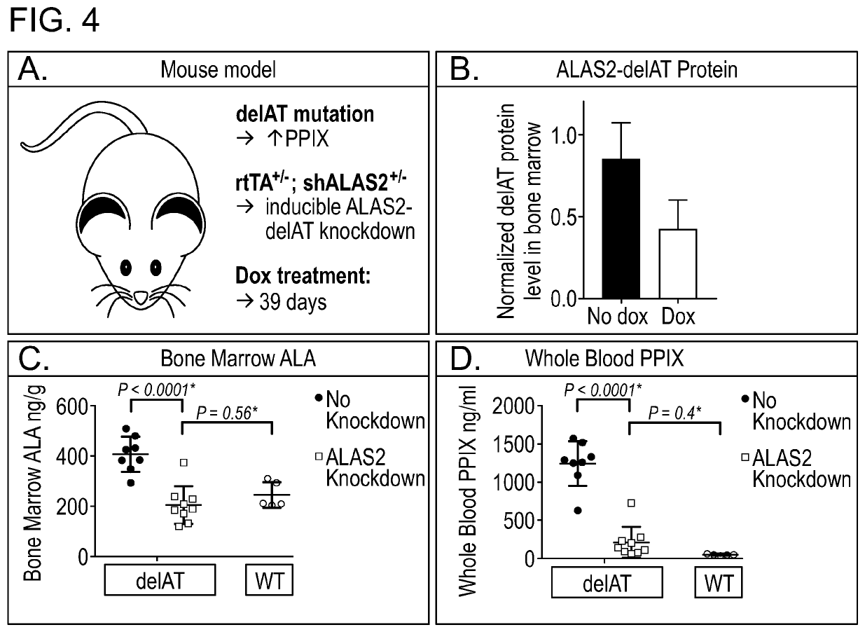

FIG. 5 depicts the exemplified compounds that inhibited ALAS2 in erythroporphyria cell models in vitro. The exemplified compounds blocked PPIX production in an engineered cell model mimicking EPP (Example 36.5). The exemplified compounds dose-dependently reduced PPIX fluorescence without affecting cell viability at concentrations tested. Compound E1 was also tested in an in vitro XLP cell model. Compound E1 blocked PPIX production in ex vivo differentiating erythroprogenitor cells isolated from XLP mice with ALAS2-delAT mutation. Compound E1 dose-dependently reduced PPIX fluorescence without affecting cell viability at the concentrations tested. Thus, the data suggest the compounds as provided herein can be used to treat erythroid porphyrias by normalizing PPIX level.

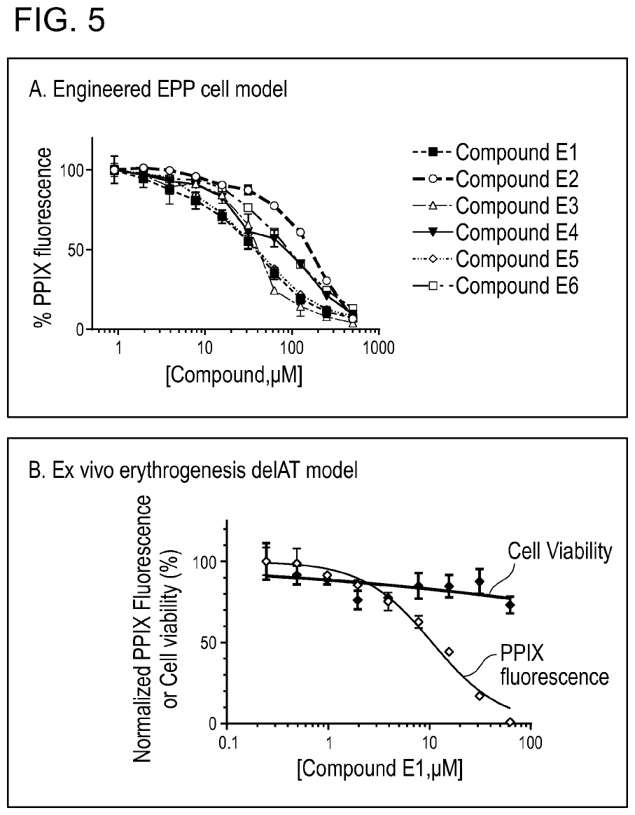

FIG. 6 depicts pharmacological proof of mechanism of ALAS2 inhibition strategy in vivo (Example 36.6). Compound E1 potently inhibited ALAS2 with delAT mutation in the XLP mouse model leading to reduction in ALA and PPIX in the bone marrow. Compound E1 was dosed in the delAT mice by oral gavage and observed compound accumulation in the bone marrow compartment and potent reduction in bone marrow ALA 30 mins after dosing. Consequently, a reduction of total bone marrow PPIX was also observed. Thus, the data provided pharmacological proof of mechanism of ALAS2 inhibition strategy in vivo to treat a erythroid porphyria.

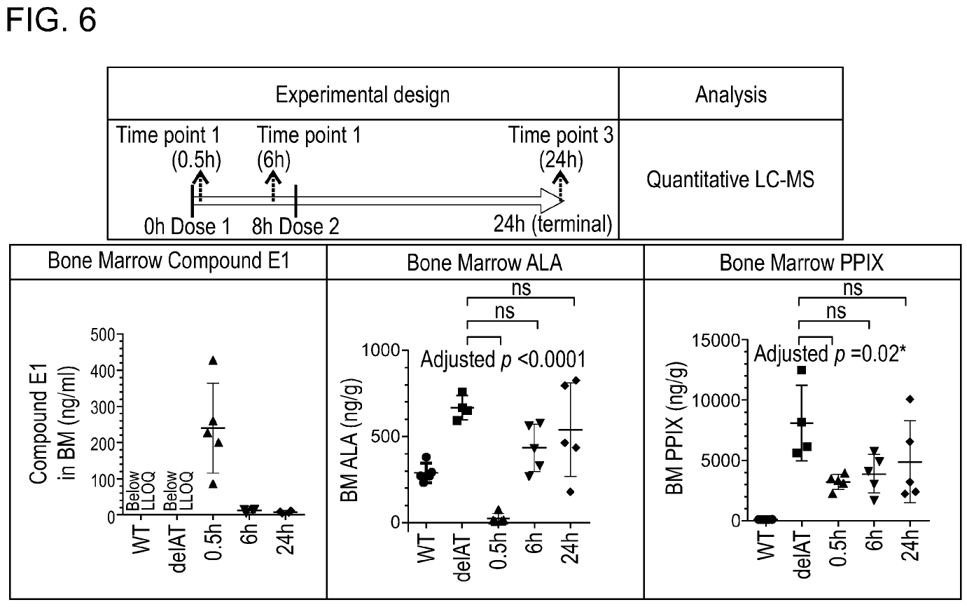

FIG. 7 depicts the structures of the exemplified compounds tested in Example 36, FIGS. 5 and 6.

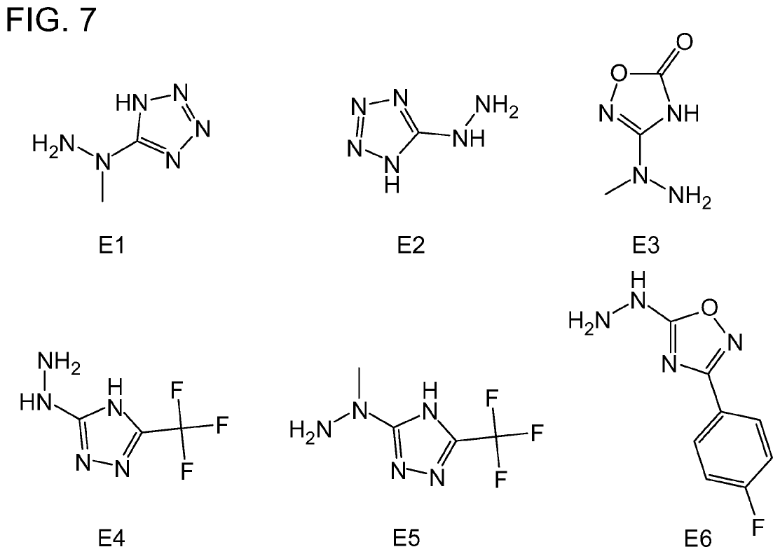

FIG. 8 depicts the sequence of ALAS2 (SEQ ID NO:3).

### DETAILED DESCRIPTION

### Compounds

Described herein are compounds that are useful for treating various diseases, disorders and medical conditions, including those characterized by defects in the heme pathway that lead to accumulation of one or more metabolites of the pathway. Inhibition of the enzyme in the pathway, namely ALAS2, as shown in Example 36, may reduce the level of accumulated metabolites, thereby alleviating the symptoms of the disorder. Examples of disorders treatable with the compounds of the invention are but not limited to X-linked protoporphyria, erythropoietic protoporphyria, congenital erythropoietic protoporphyria, myelodysplastic syndrome associated with isolated del(5q) (Del5q MDS), and Diamond-Blackfan anemia (DBA).

In a first embodiment, a compound of the present invention is a compound of Formula (I) or a pharmaceutically acceptable salt thereof as described in the summary.

In a second embodiment, in a compound of Formula (I) or a pharmaceutically acceptable salt thereof, Ring A is a 5-membered heteroaryl containing 1-4 instances of N; wherein the remaining variables are as defined in the first embodiment.

In a third embodiment, in a compound of Formula (I) or a pharmaceutically acceptable salt thereof, Ring A is a 5-membered heteroaryl containing 1-2 instances of N and one instance of S or O; wherein the remaining variables are as defined in the first or second embodiments.

In a fourth embodiment, in a compound of Formula (I) or a pharmaceutically acceptable salt thereof, Ring A is 1,2,4-oxadiazolyl, pyrazolyl, 1,2,4-triazolyl, tetrazolyl, 1,2,3-thiadiazolyl, thiazolyl, 1,3,4-thiadiazolyl, 1,2,4-oxadiazolyl-5(4H)-one, 1,2,3-triazolyl, 1,2,4-triazolyl-3-one, ¹H-imidazolyl, 1,3,4-oxadiazolyl, or oxazolyl; wherein the remaining variables are as defined in the first, second, or third embodiments.

In a fifth embodiment, in a compound of Formula (I) or a pharmaceutically acceptable salt thereof, Ring A is selected from one of the following:

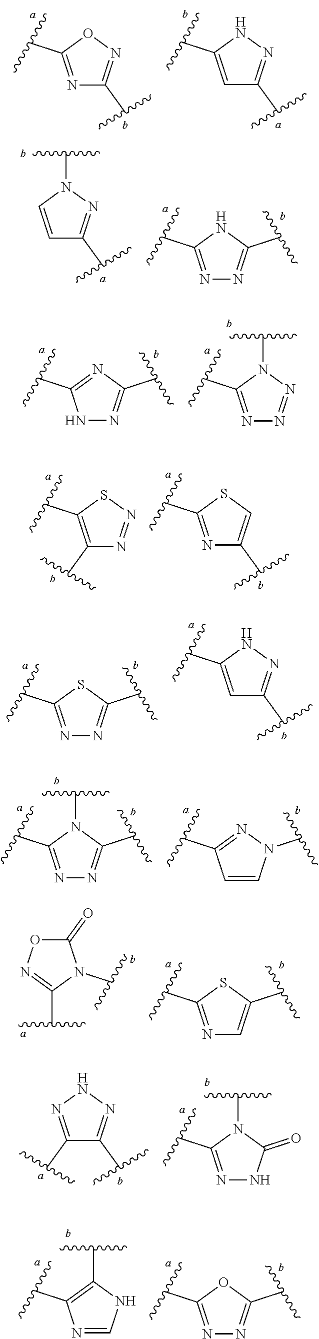

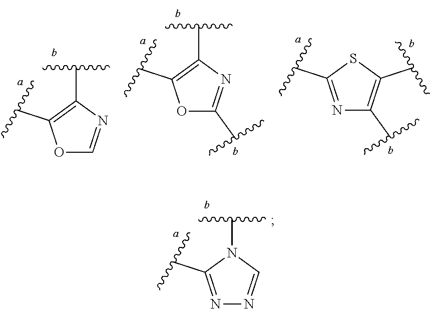 [SMILES: *c1ncoc1* ; *c1nc(*)c(*)o1 ; *c1nc(*)c(*)s1 ; *c1nncn1*]

wherein the remaining variables are as defined in the first, second, third, or fourth embodiments.

In a sixth embodiment, in a compound of Formula (I) or a pharmaceutically acceptable salt thereof, R¹ is hydrogen or C₁-C₆ alkyl; wherein the remaining variables are as defined in the first, second, third, fourth, or fifth embodiments.

In a seventh embodiment, in a compound of Formula (I) or a pharmaceutically acceptable salt thereof, R¹ is hydrogen, methyl, ethyl, propyl, i-propyl, cyclopropyl, —C(═O)CF3, or —CH₂CH₂OH; wherein the remaining variables are as defined in the first, second, third, fourth, or fifth embodiments.

In an eighth embodiment, in a compound of Formula (I) or a pharmaceutically acceptable salt thereof, R² is hydrogen, —CN, hydroxyl, C₁-C₆ alkyl, C₁-C₆ haloalkyl, —CH₂OCH₃, —CH₂-phenyl, phenyl, o-methylphenyl, p-aminophenyl, p-methoxylphenyl, p-fluorophenyl, naphthalyl, cyclopropyl, —CO₂H, or —CO₂Et; wherein the remaining variables are as defined in the first, second, third, fourth, fifth, sixth, or seventh embodiments.

In a ninth embodiment, in a compound of Formula (I) or a pharmaceutically acceptable salt thereof, R² is 5- or 6-membered monocyclic heteroaryl, 8-12 membered bicyclic heteroaryl, or 3-14 membered heterocyclyl; wherein the remaining variables are as defined in the first, second, third, fourth, fifth, sixth, or seventh embodiments.

In a tenth embodiment, in a compound of Formula (I) or a pharmaceutically acceptable salt thereof, R² is pyridinyl, furanyl, or morpholinyl; wherein the remaining variables are as defined in the first, second, third, fourth, fifth, sixth, seventh, or ninth embodiments.

In an eleventh embodiment, in a compound of Formula (I) or a pharmaceutically acceptable salt thereof, n is 0, 1 or 2; wherein the remaining variables are as defined in the first, second, third, fourth, fifth, sixth, seventh, eighth, ninth, or tenth embodiments.

In a twelfth embodiment, a compound of the present invention or a compound of Formula (I) is compound of Table lor a pharmaceutically acceptable salt thereof:

TABLE 1
Exemplary compounds of Formula (I)

| Cpd | |
|---|---|
| Nr | Structure |
| 1 | 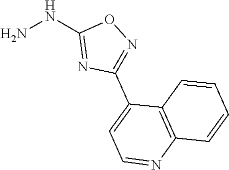 [SMILES: NNc1nc(-c2ccnc3ccccc23)no1] |
| 2 | 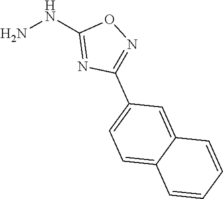 [SMILES: NNc1nc(-c2ccc3ccccc3c2)no1] |
| 3 | 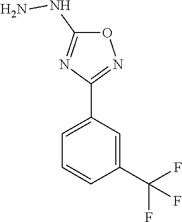 [SMILES: NNc1nc(-c2cccc(C(F)(F)F)c2)no1] |
| 4 | 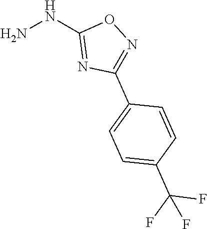 [SMILES: NNc1nc(-c2ccc(C(F)(F)F)cc2)no1] |
| 5 | 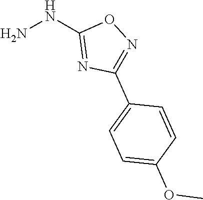 |
| 6 | 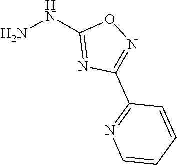 [SMILES: NNc1nc(-c2ccccn2)no1] |
| 7 | 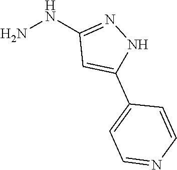 [SMILES: NNc1cc(-c2ccncc2)[nH]n1] |
| 8 | 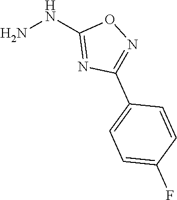 [SMILES: NNc1nc(-c2ccc(F)cc2)no1] |
| 9 | 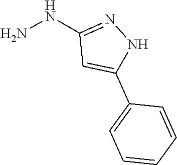 [SMILES: NNc1cc(-c2ccccc2)[nH]n1] |
| 10 | 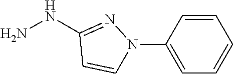 [SMILES: NNc1ccn(-c2ccccc2)n1] |
| 11 | 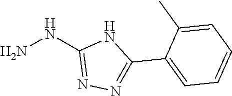 |
| 12 | 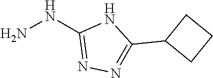 [SMILES: NNc1nnc(C2CCC2)[nH]1] |
| 13 | 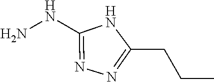 |
| 14 | 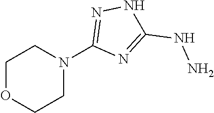 [SMILES: NNc1nc(N2CCOCC2)n[nH]1] |
| 15 | 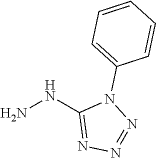 [SMILES: NNc1nnnn1-c1ccccc1] |
| 16 | 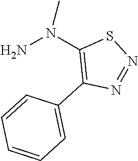 |
| 17 | 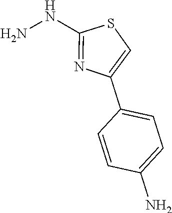 [SMILES: NNc1nc(-c2ccc(N)cc2)cs1] |
| 18 | 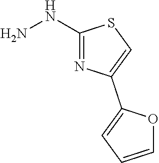 [SMILES: NNc1nc(-c2ccco2)cs1] |
| 19 | 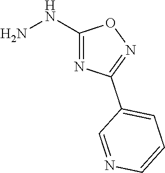 [SMILES: NNc1nc(-c2cccnc2)no1] |
| 20 | 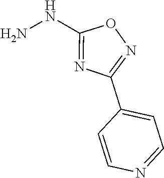 [SMILES: NNc1nc(-c2ccncc2)no1] |
| 21 | 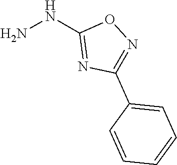 [SMILES: NNc1nc(-c2ccccc2)no1] |
| 22 | 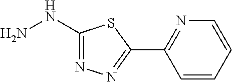 [SMILES: NNc1nnc(-c2ccccn2)s1] |
| 23 | 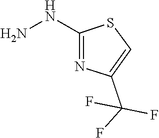 [SMILES: NNc1nc(C(F)(F)F)cs1] |
| 24 | 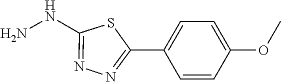 |
| 25 | 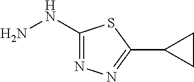 |
| 26 | 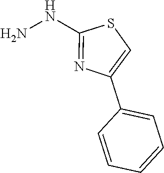 [SMILES: NNc1nc(-c2ccccc2)cs1] |
| 27 | 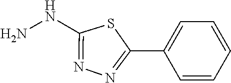 [SMILES: NNc1nnc(-c2ccccc2)s1] |
| 28 | 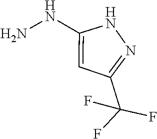 [SMILES: NNc1cc(C(F)(F)F)n[nH]1] |
| 29 | 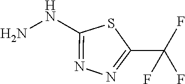 [SMILES: NNc1nnc(C(F)(F)F)s1] |
| 30 | 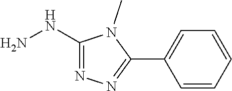 |
| 31 | 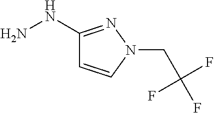 [SMILES: NNc1ccn(CC(F)(F)F)n1] |
| 32 | 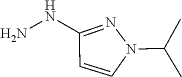 |
| 33 | 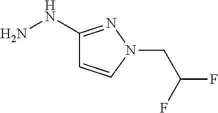 [SMILES: NNc1ccn(CC(F)F)n1] |
| 34 | 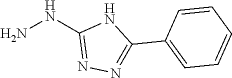 [SMILES: NNc1nnc(-c2ccccc2)[nH]1] |
| 35 | 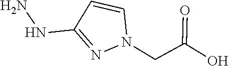 [SMILES: NNc1ccn(CC(=O)O)n1] |
| 36 | 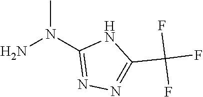 |
| 37 | 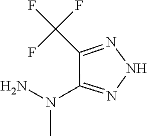 |
| 38 | 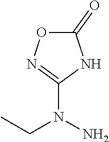 |
| 39 | 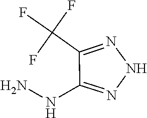 [SMILES: NNc1n[nH]nc1C(F)(F)F] |
| 40 | 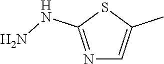 |
| 41 | 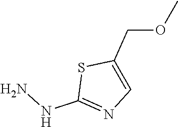 |
| 42 | 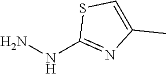 |
| 43 | 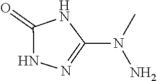 |
| 44 | 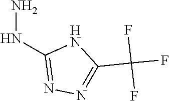 [SMILES: NNc1nnc(C(F)(F)F)[nH]1] |
| 45 | 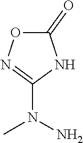 |
| 46 | 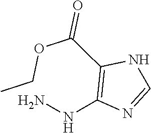 |
| 47 | 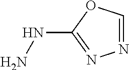 |
| 48 | 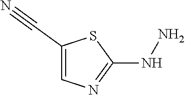 |
| 49 | 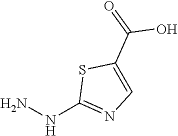 |
| 50 | 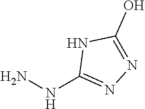 |
| 51 | 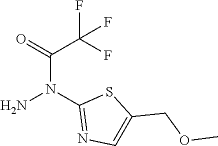 [SMILES: COCc1cnc(N(N)C(=O)C(F)(F)F)s1] |
| 52 | 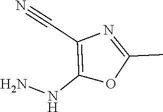 |
| 53 | 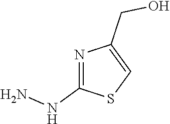 |
| 54 | 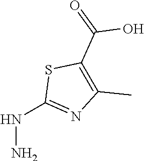 |
| 55 | 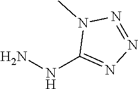 |
| 56 | 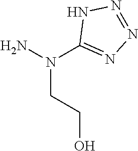 |
| 57 | 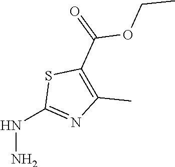 |
| 58 | 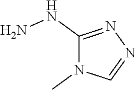 |
| 59 | 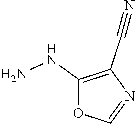 |
| 60 | 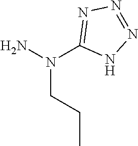 |
| 61 | 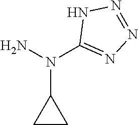 |
| 62 | 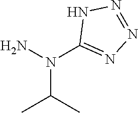 |
| 63 | 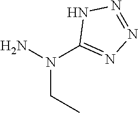 |
| 64 | 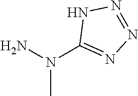 |
| 65 | 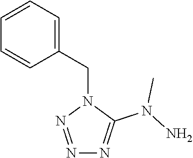 |
| 66 | 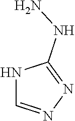 |
| 67 | 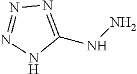 |

The exemplified compounds in Table I were tested in vitro against the wild type ALAS2. As described in Example 38, two concentrations of ALAS2 were used: 5 ug/mL ALAS2 (for the version I assay) and 2 ug/mL (for the version 2 assay). “A” refers to an IC₅₀ less than 10.0 μM; “B” refers to an ICso from 10.0 μM to 50.0 μM; and “C” refers to an AC₅₀ greater than 50.0 μM.

TABLE 2
Biochemical activities of the exemplified compounds in Table 1.

| | IC50 (uM) | IC50 (uM) |
|---|---|---|
| Compound No. | ALAS2 v1; 0 uM PLP | ALAS2 v2; 0 uM PLP |
| 1. | | A |
| 2. | | A |
| 3. | | C |
| 4. | | B |
| 5. | | B |
| 6. | B | |
| 7. | B | |
| 8. | B | |
| 9. | B | |
| 10. | B | |
| 11. | A | |
| 12. | C | |
| 13. | B | |
| 14. | B | |
| 15. | C | |
| 16. | No Fit | |
| 17. | C | |
| 18. | C | |
| 19. | A | |
| 20. | A | |
| 21. | A | |
| 22. | A | |
| 23. | C | |
| 24. | B | |
| 25. | C | |
| 26. | C | |
| 27. | A | |
| 28. | B | |
| 29. | A | |
| 30. | C | |
| 31. | C | |
| 32. | A | |
| 33. | B | |
| 34. | A | |
| 35. | A | |
| 36. | A | |
| 37. | B | |
| 38. | A | |
| 39. | A | |
| 40. | A | |
| 41. | C | |
| 42. | C | |
| 43. | A | |
| 44. | A | |
| 45. | A | |
| 46. | A | |
| 47. | C | |
| 48. | B | |
| 49. | C | |
| 50. | C | |
| 51. | C | |
| 52. | C | |
| 53. | B | |
| 54. | B | |
| 55. | C | |
| 56. | C | |
| 57. | B | |
| 58. | C | |
| 59. | C | |
| 60. | C | |
| 61. | C | |
| 62. | No Fit | |
| 63. | C | |
| 64. | A | |
| 65. | C | |
| 66. | A | |
| 67. | A | |

The term “alkyl” refers to a radical of a straight-chain or branched saturated hydrocarbon group having from 1 to 12 carbon atoms, e.g., 1 to 6 carbon atoms (“C₁-C₆ alkyl”). Examples of C₁-C₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl).

The term “halo” or “halogen” refers to fluorine, chlorine, bromine, or iodine.

The term “haloalkyl” refers to an alkyl group as defined above, such as a C₁-C₆ alkyl (“C₁-C₆ haloalkyl”), wherein one or more of the hydrogen atoms are independently replaced by a halogen, e.g., fluoro, bromo, chloro, or iodo and includes alkyl moieties in which all hydrogens have been replaced by halo (e.g., perfluoroalkyl).

The term “alkoxy” refers to an —O-alkyl radical group, wherein alkyl is as defined above, e.g., with between 1 and 6 carbon atoms (“C₁-C₆ alkoxy”).

The term “acyl” refers to a radical group having the general formula —C(═O)R^{X1}, wherein R^{X1} is an alkyl group as defined above.

The term “cycloalkyl” as employed herein includes saturated cyclic, bicyclic, tricyclic, or polycyclic (e.g., a fused, bridged or spiro ring system) hydrocarbon groups having 3 to 14 carbons containing the indicated number of rings and carbon atoms (e.g., a C₃-C₈ cycloalkyl).

The term “heterocyclyl” or “heterocyclic” refers to a radical of a 3- to 14-membered saturated or unsaturated, and non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”), and can be saturated or can contain one or more carbon-carbon double bonds. Heterocyclyl polycyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). Exemplary heterocyclyl groups include aziridinyl, oxiranyl, thiiranyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, pyrrolyl-2,5-dine, dioxolanyl, oxathiolanyl, dithiolanyl, triazolinyl, oxadiazolinyl, thiadiazolinyl,piperidinyl, tetrahydropyranyl, dihydropyridinyl, thianyl, piperazinyl, morpholinyl, dithianyl, dioxanyl, triazinanyl, azepanyl, oxepanyl, thiepanyl, azocanyl, oxecanyl, thiocanyl, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, ¹H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6, 7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “heteroaryl” refers to a radical of a 5-14 membered monocyclic or polycyclic (e.g., bicyclic, tricyclic, etc.) 4n+2 aromatic ring system (e.g., having 6, 10, or 14 x electrons shared in a cyclic array) having ring carbon atoms and 1-4 ring heteroatoms provided in the aromatic ring system, wherein each heteroatom is independently selected from nitrogen, oxygen, and sulfur. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continues to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). In certain embodiments, the heteroaryl is a monocyclic heteroaryl, such as a 5- or 6-membered monocyclic heteroaryl. Exemplary monocyclic heteroaryl groups include pyrrolyl, furanyl, thiophenyl, imidazolyl, pyrazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, triazinyl, tetrazinyl, azepinyl, oxepinyl, thiepinyl, 1,2,4-oxadiazolyl, 1,2,4-triazolyl, tetrazolyl, 1,2,3-thiadiazolyl, 1,3,4-thiadiazolyl, 1,2,4-oxadiazolyl-5(4H)-one, 1,2,3-triazolyl, 1,2,4-triazolyl-3-one, ¹H-imidazolyl, or 1,3,4-oxadiazolyl etc. In certain embodiments, the heteroaryl is a bicyclic heteroaryl, such as a 8-12 membered bicyclic heteroaryl. Exemplary bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzothiazolyl, benzisothiazolyl, benzthiadiazolyl, indolizinyl, purinyl, naphthyridinyl, pteridinyl, quinolinyl, isoquinolinyl, cinnolinyl, quinoxalinyl, phthalazinyl, etc .

The term “aryl” refers to a radical of a monocyclic or polycyclic (e.g., bicyclic or tricyclic, etc.) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more cycloalkyl or aryl groups. A phenyl substituted with methyl, amine, methoxy, or fluoro is described herein as methylphenyl, aminophenyl, methoxyphenyl, or fluorophenyl, respectively.

The term “fused” referring to a bicyclic, tricyclic or polycyclic ring system as used herein, is a bicyclic, tricyclic or polycyclic ring system wherein at least two rings share two adjacent ring atoms selected from C, N, O and S.

The term “spiro” referring to a bicyclic, tricyclic or polycyclic ring system as used herein, is a bicyclic, tricyclic or polycyclic ring system wherein at least two rings share one ring atom that is selected from C, N, O and S.

The term “bridged” referring to a bicyclic, tricyclic or polycyclic ring system as used herein, is a bicyclic, tricyclic or polycyclic ring system wherein at least two rings are two non-adjacent ring atoms selected from C, N, O and S.

The term “pharmaceutically acceptable carrier or adjuvant” refers to a carrier or adjuvant that may be administered to a patient, together with a compound provided herewith, and which does not destroy the pharmacological activity thereof and is nontoxic when administered in doses sufficient to deliver a therapeutic amount of the compound.

### Treatment of Disorders

In another aspect, the present disclosure is directed to a method of treating a subject having a disorder in need of treatment, comprising inhibiting aminolevulinic acid synthase 2 (ALAS2) in the subject by administering a compound of Formula (I) or a pharmaceutically acceptable salt thereof as defined above in the first through twelfth embodiments; use of a compound of Formula (I) or a pharmaceutically acceptable salt thereof as defined above in the first through twelfth embodiments for the manufacture of a medicament for treating a disorder; and a compound of Formula (I) or a pharmaceutically acceptable salt thereof as defined above in the first through twelfth embodiments for use in treating a disorder.

Disorders that can be treated with a compound of Formula (I) or a pharmaceutically acceptable salt thereof are characterized by defects in the heme pathway that lead to accumulation of one or more physiological or non-physiological metabolites of the pathway. Examples of these metabolites of the heme pathway are 5-aminolevulinic acid, 8-aminolevulinic acid (δALA), porphobilinogen (BPG), hydroxymethylbilane (HMB), uroporphyrinogen I, uroporphyrinogen III (UROgen III), coproporphyrinogen I, coproporphyrinogen III (CPgenIII), protoporphyrinogen IX, uroporphyrin I, coproporphyrin I, heme, and protoporphyrin IX (PPIX).

The defects in the heme pathway are attributed to the deregulation of one or more enzymes in the heme pathway. The enzymes in the heme pathway include 8-aminolevulinic acid synthase (ALAS2), ALA dehydratase (ALAD), hydroxymethylbilane synthase (HMBS), uroporphyrinogen III synthase (UROS), uroporphyrinogen decarboxylase (UROD), coproporphyrinogen oxidase (CPOX), protoporphyrinogen oxidase (PPIOX), and ferrochelatase (FECH). Examples of metabolites of the heme pathway are 5-aminolevulinic acid, 8-aminolevulinic acid (δALA), porphobilinogen (BPG), hydroxymethylbilane (HMB), uroporphyrinogen I, uroporphyrinogen III (UROgen III), coproporphyrinogen I, coproporphyrinogen III (CPgenIII), protoporphyrinogen IX, uroporphyrin I, coproporphyrin I, heme, and protoporphyrin IX (PPIX).

In one embodiment, the disorder treated in a method of invention is associated with deregulated wild-type ALAS2. In some embodiments, the deregulated ALAS2 is associated with an increase in the amount of ALAS2, and/or at least one metabolite from the heme pathway as described above, for example at least one of uroporphyrin I, coproporphyrin I, heme, and protoporphyrin IX (PPIX).

Alternatively, the deregulated ALAS2 is a mutated ALAS2 that is associated with increased ALAS2 activity. To determine if a subject's disorder is associated with a wild-type ALAS2 or a mutated ALAS2 with increased activity, a method of the invention further comprises a step of determining the nucleic acid and/or amino acid sequence the subject's ALAS2 and comparing to the wild-type enzyme.

Additional disorders that can be treated with a compound of Formula (I) or a pharmaceutically acceptable salt thereof include disorders that are caused by mutations in one or more enzymes in the heme pathway (see examples of the enzymes given above). In one embodiment, the mutated enzyme is ALAS2. In another embodiment, the mutated enzyme is FECH. In yet another embodiment, the mutated enzyme is UROS. One class of such disorders are blood disorders known as “porphyria”, “erythroporphyria” or “erythoid porphyria”. Porphyrias are characterized by acute photosensitivity resulting in painful attacks, due to pathologically elevated or accumulated erythrocyte porphyrins. At least three subtypes of porphyria are known: X-linked protoporphyria, erythropoietic protoporphyria, and congenital erythropoietic porphyria.

“X-linked proporphyria” (XPP) is caused by gain-of-function mutations in δ-aminolevulinic acid synthase isoform 2 ALAS2 (first enzyme of the heme pathway). These mutations increase ALAS2 enzymatic activity, leading to an increased pathway flux to such an extend that overwhelms the capacity of the last enzyme in the pathway which is ferrochelatase (FECH). As a result, the penultimate pathway intermediate, protoporphyrin IX (PPIX), cannot be processed and thus accumulates in the body.

“Erythropoietic proporphyria” (EPP) is caused by loss-of-function mutations in FECH. Reduction in FECH activity creates a bottleneck that also results in accumulation of PPIX.

PPIX, uroporphyrin I, and coproporphyrin I can be the molecular culprits of these erythroporphyrias. These molecules are photo-excited when exposed to light in the skin, generating reactive species that cause intense pain and other features characteristics of these diseases. Therefore, reduction of these metabolites may help ameliorate the erythroporphyrias.

Myelodysplastic syndrome associated with isolated del(5q) (Del5q MDS) or Diamond-Blackfan anemia (DBA) are rare disorders caused by defects in ribosomal proteins, which are important in protein translation. Heme production and globin protein synthesis are highly coordinated events during red cell maturation. However, due to the defects in ribosomal protein functions, the translation of globin proteins in Del5q MDS and DBA can be outpaced by the synthesis of heme, resulting in accumulation of toxic level of heme, which in turn inhibits erythropoiesis. Thus, the methods provided herein inhibiting heme production may help treating Del5q MDS or DBA.

“Congenital erythropoietic protoporphyria” (CEP) is caused by loss-of-function mutations in uroporphyrinogen III synthase (UROS). Normally, UROS converts hydroxymethylbilane to uroporphyrinogen III, which is a physiological and direct product that can be further metabolized. Mutations of UROS cause a blockade at this enzymatic step, leading to accumulation of hydroxymethylbilane. Accumulated hydroxymethylbilane can undergo a non-enzymatic reaction to form uroporphyrinogen I and eventually uroporphyrin I or coproporphyrin I, which are both non-physiological and dead-end products that cannot be further metabolized, thereby resulting in their accumulation in the body.

Another class of disorders that can be treated with a compound of Formula (I) or a pharmaceutically acceptable salt thereof is “anemia”, which is a disorder associated with a deficiency of red blood cells (RBCs) and/or hemoglobin. In one embodiment, the anemia is further associated with deregulated ALAS2, examples of which include Diamond-Blackfan anemia and X-sideroblastic anemia.

Yet another group of disorders that can be treated with a compound of Formula (I) or a pharmaceutically acceptable salt thereof is are disorders that are caused by defects in ribosomal proteins. Due to defects in ribosomal protein functions, the translation of globin proteins these disorders is outpaced by heme synthesis, thereby resulting in accumulation of a toxic levels of heme, which in erythropoiesis (i.e., production of RBCs). Examples of disorders that are caused by the defects in ribosomal proteins are in myelodysplastic syndrome (MDS) with isolated del(5q) and Diamond-Blackfan anemia.

### Combining ALAS2 Inhibitor Treatment with Standard Therapies

In certain embodiments, the subject is concomitantly receiving or has received β-carotene therapy, porphyrin absorbent therapy, afamelanotide treatment, or blood transfusion.

Oral beta-carotene therapy includes Lumitene and Tishcon to improve an affected individual's tolerance of sunlight. Porphyrin absorbent therapy includes cholestyramine and activated charcoal.

The disclosed methods can also be used in combination with Scenesse (afamelanotide), which has been approved for the treatment of erythropoietic protoporphyria in Europe and works by increasing skin pigmentation to provide protection and improves sun tolerance.

To treat iron deficiency, the disclosed methods can be combined with iron supplements. In addition, the disclosed methods can be combined with cholestyramine or activated charcoal to interrupt the circulation of protoporphyrin through the liver and intestines in patients with liver disease.

Individuals with high levels of protoporphyrin in the plasma and red blood cells are at risk of liver malfunction that could eventually lead to liver failure. Liver transplantation has been performed as a life-saving measure in patients with EPP and XLP related liver failure. Bone marrow transplant can also be performed after liver transplant to prevent further damage to the liver. The disclosed methods can be used with subjects who have undergone liver and/or bone marrow transplants.

### Animal (Mouse) Model of XLP

X-linked protoporphyria (XLP) (see NCBI MIM 300752, incorporated herein by reference) is an erythropoietic porphyria due to gain-of-function mutations in the erythroid-specific aminolevulinate synthase gene (ALAS2). Two previously identified exon 11 small deletions, namely c. 1699_1670ΔAT (ΔAT) and c.1706_1709ΔAGTG (AAGTG), have been identified to prematurely truncate or elongate the wild-type ALAS2 polypeptide, leading to increased ALAS2 enzymatic activity of about 20- to 40-fold, thus causing the erythroid accumulation of protoporphyrins, cutaneous photosensitivity, and liver disease. Three additional mutations, a frameshift mutation cause by 26 bp deletion (c. 1651-1677del26bp), c. 1734ΔG (ΔG), and c. 1642C>T (p.Q548X, a nonsense mutation), as well as an engineered deletion mutation, c. 1670-1671TC>GA p.F557X, were also expressed and characterized (Ducamp et al., Human Molecular Genetics 22(7): 1280-1288, 2013; Bishop et al., Mol. Med. 19(1): 18-25, 2013). According to Bishop, compared to the purified wild-type enzyme, ΔAT, ΔAGTG and Q548X enzymes had increased specific activities that were 1.8-, 3.1- and 1.6-fold, respectively. Meanwhile, the elongated ΔG enzyme had wild-type specific activity, kinetics and thermostability; but twice the wild-type purification yield (56 versus 25%); suggesting greater stability in vivo. On the basis of studies of mutant enzymes, the maximal gain-of function region spanned 57 amino acids between 533 and 580. Overall, these ALAS2 gain-of-function mutations increased the specific activity (ΔAT, ΔAGTG and p.Q548X) or stability (ΔG) of the enzyme, thereby leading to the increased erythroid protoporphyrin accumulation causing XLP.

Thus, another aspect of the invention provides a mouse (Mus musculus) as an animal model for X-linked protoporphyria (XLP), wherein the mouse comprises a genomic ALAS2 (5-AminoLevulinic Acid Synthase 2)-gain-of-function (gof) mutation at the mouse ALAS2 locus, wherein the genomic ALAS2-gof mutation encodes a mutant ALAS2 protein with increased activity and/or stability over a wild-type ALAS2 protein, and corresponds to or recapitulates a human ALAS2-gof mutation in an XLP human patient.

In certain embodiments, the genomic ALAS2-gof mutation corresponds to or recapitulates a human ALAS2-gof mutation selected from the group consisting of: c. 1651-1677del26bp, c. 1699_1670ΔAT (ΔAT); c.1706_1709ΔAGTG (ΔAGTG); c. 1734AG (AG); c. 1642C>T (p.Q548X); and c. 1670-1671TC>GA p.F557X.

In certain embodiments, the genomic ALAS2-gof mutation corresponds to or recapitulates a human ALAS2-gof nonsense mutation selected from the group consisting of: p.G544X and p.G576X.

According to this aspect of the invention, the endogenous mouse ALAS2 gene is modified to mimic the genetic gof mutation found in a human XLP patient, such as the human ALAS2-delAT (c.1699_1670ΔAT) mutation in an XLP human patient, in which the dinucleotides AT at nt. 1699 and 1670 are deleted. The mutation can be introduced into a mouse zygote using any art-recognized means, such as CRISPR/Cas-mediated gene editing, or introduced into a mouse ES (embryonic stem) cell via traditional homologous recombination.

Specifically, in one embodiment, the mouse (Mus musculus) is an animal model for X-linked protoporphyria (XLP), wherein the mouse comprises a genomic ALAS2 (5-AminoLevulinic Acid Synthase 2)-delAT mutation at the mouse ALAS2 locus, wherein the genomic ALAS2-delAT mutation encodes a mutant ALAS2 protein with a C-terminal deletion, and corresponds to or recapitulates the human ALAS2-delAT (c.1699_1670AAT) mutation in an XLP human patient (e.g., the mouse has the identical C-terminal protein deletion seen in human XLP patients with the delAT mutation).

In certain embodiments, the mouse has elevated level of blood protoporphyrin IX (PPIX) and Zn-PPIX compared to a syngeneic wild-type mouse.

In certain embodiments, the mouse is an inbred strain of mouse, such as C₅₇BL/6 mouse.

In certain embodiments, the mouse is a male, which is hemizygous for the mutation on the X chromosome (ALAS2^{delAT/Y}).

In certain embodiments, the mouse is a female, which can be heterozygous or homozygous for the mutation. In particular, in certain embodiments, the mouse is homozygous for the genomic ALAS2-delAT mutation (ALAS2^{delAT/delAT}). In certain other embodiments, the mouse is heterozygous for the genomic ALAS2-delAT mutation (i.e., the female mouse comprises one wild-type allele of ALAS2) (ALAS2^{delAT/WT}).

In certain embodiments, the mouse comprises the genomic DNA sequence of SEQ ID NO: 5, which comprises silent point mutations in addition to the mutations responsible for the gof mutant phenotype.

In certain embodiments, the mouse is generated by CRISPR/Cas9-mediated homology-directed repair (HDR) that deletes the AT dinucleotide in the genomic ALAS2-delAT mutation.

In certain embodiments, the CRISPR/Cas9-mediated HDR is carried out by microinjecting into the pronucleus of a mouse zygote an mRNA encoding Cas9, an sgRNA, and a single-stranded DNA (ssDNA) (e.g., all 10 ng/μL).

In particular, a CRISPR/Cas9 coding sequence (e.g., Cas9 mRNA), its single guide RNA (sgRNA) targeting an ALAS2 sequence for generating a double-stranded break (DSB) to facilitate homology-directed repair (HDR) using a donor DNA (e.g., a single-stranded donor DNA) harboring the desired sequence change to introduce the mutation (e.g., the AT dinucleotide deletion), and the donor DNA, can be introduced together into the nucleus of a mouse zygote by, for example, microinjection or eletroporation. Upon synthesis of the Cas9 enzyme in the zygote, the sgRNA is loaded onto the Cas9 effector enzyme to generate a DSB, which can be repaired by HDR using the sequence in the donor DNA.

The zygote (e.g., a zygote for a male mouse) having such mutation on the X chromosome is then allowed to develop to term in a surrogate female to generate the founder male mouse and the female mouse (which can be homozygous or heterozygous for the mutation). Subsequent crossing of the male and/or female founders to the background strain produces female progenies that are homozygous or heterozygous and male progenies that are hemizygous for the mutation. The presence of mutations can be verified through genotyping using standard technology, such as PCR using genomic DNA isolated from tails and toes.

In certain embodiments, the single guide RNA (sgRNA) for CRISPR/Cas9 comprises the nucleotide sequence of SEQ ID NO: 4.

In certain embodiments, the CRISPR/Cas9-mediated HDR utilizes a donor DNA having the polynucleotide sequence of SEQ ID NO: 5.

In certain embodiments, the CRISPR/Cas9-mediated HDR utilizes a donor DNA from an XLP patient (e.g., a mouse or a human patient).

### Other Definitions

The terms “effective amount” and “therapeutically effective amount” are used interchangeably throughout the present disclosure, when referring to a compound of Formula (I) or a pharmaceutically acceptable salt thereof.

An “effective amount” of a compound of Formula (I) or a pharmaceutically acceptable salt thereof is an amount sufficient to provide a therapeutic benefit in the treatment of a disorder, such a disorder associated with deregulated ALAS2. Additionally or alternatively, an “effective amount” of a compound of Formula (I) or a pharmaceutically acceptable salt thereof is an amount sufficient to delay or reduce one or more effects or symptoms associated with these disorders. In one aspect, an “effective amount” of a compound of Formula (I) or a pharmaceutically acceptable salt thereof, alone or in combination with other therapies, which provides a therapeutic benefit in the treatment of the disorder. The term “effective amount” can encompass an amount that improves overall therapy, reduces or avoids effects, symptoms, signs, or causes of the condition, and/or enhances the therapeutic efficacy of another therapeutic agent. In certain embodiments, an “effective amount” of a compound of Formula (I) or a pharmaceutically acceptable salt thereof is an amount sufficient for eliciting measurable inhibition of ALAS2. In certain embodiments, an “effective amount” of a compound of Formula (I) or a pharmaceutically acceptable salt thereof is an amount sufficient for degrading or inhibiting ALAS2 in a subject in need thereof. In certain embodiments, an “effective amount” of a compound of Formula (I) or a pharmaceutically acceptable salt thereof is an amount sufficient for reducing the amount of at least one metabolite selected from the group consisting of 5-aminolevulinic acid, 8-aminolevulinic acid (8ALA), porphobilinogen (BPG), hydroxymethylbilane (HMB), uroporphyrinogen I, uroporphyrinogen III (UROgen III), coproporphyrinogen I, coproporphyrinogen III (CPgenIII), protoporphyrinogen IX, uroporphyrin I, coproporphyrin I, heme, and protoporphyrin IX (PPIX). In one aspect, the effective amount of a compound of Formula (I) or a pharmaceutically acceptable salt thereof is between about 0.01-100 mg/kg body weight/day of the compound.

The terms “treatment,” “treat,” and “treating” refer to reversing, alleviating, reducing the likelihood of developing, or inhibiting the progress of a disease or disorder, or one or more effects or symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), i.e., prophylactic treatment. Treatment may also be continued after symptoms have resolved, for example to reduce the likelihood of or delay their recurrence.

As used herein the terms “subject” and “patient” may be used interchangeably, and means a mammal or a human in need of treatment with a compound of Formula (I) or a pharmaceutically acceptable salt thereof.

The terms “composition” and “formulation” are used interchangeably.

The compositions delineated herein include the compounds delineated herein (e.g., a compound of Formula (I) or a pharmaceutically acceptable salt thereof described herein), as well as additional therapeutic agents if present, in amounts effective for achieving a modulation of disease or disease symptoms, including those described herein.

Pharmaceutically acceptable carriers, adjuvants and vehicles that may be used in the pharmaceutical compositions provided herewith include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions provided herewith may be administered orally, parenterally, by inhalation spray, topically, rectally, nasally, buccally, vaginally or via an implanted reservoir, preferably by oral administration or administration by injection. The pharmaceutical compositions provided herewith may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions provided herewith may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

When the compositions provided herewith comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds provided herewith. Alternatively, those agents may be part of a single dosage form, mixed together with the compounds provided herewith in a single composition.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

### EXEMPLIFICATIONS

### Abbreviations List

| Abbrv. | Full Name | Abbrv. | Full Name |
|---|---|---|---|
| anhy. | anhydrous | aq. | aqueous |
| min | minute(s) | satd. | saturated |
| mL | milliliter | h or hrs | hours |
| mmol | millimole(s) | mol | mole(s) |
| MS | mass spectrometry | NMR | nuclear magnetic |
| | | | resonance |
| TLC | thin layer | HPLC | high-performance |
| | chromatography | | liquid chromatography |
| LCMS | Liquid chromatography- | nBuLi | n-butyllithium |
| | mass spectrometry | | |
| DCE | 1,2-dichloroethane | CHCl₃ | chloroform |
| DCM | dichloromethane | DMF | dimethylformamide |
| Et2O | diethyl ether | EtOH | ethyl alcohol |
| EtOAc | ethyl acetate | MeOH | methyl alcohol |
| MeCN | acetonitrile | PE | petroleum ether |
| THF | tetrahydrofuran | DMSO | dimethyl sulfoxide |
| AcOH | acetic acid | HCl | hydrochloric acid |
| H₂SO₄ | sulfuric acid | NH₄Cl | ammonium chloride |
| KOH | potassium hydroxide | NaOH | sodium hydroxide |
| K₂CO₃ | potassium carbonate | Na₂CO₃ | sodium carbonate |
| TFA | trifluoroacetic acid | Na₂SO₄ | sodium sulfate |
| NaBH₄ | sodium borohydride | NaHCO₃ | sodium bicarbonate |
| LiHMDS | lithium | NaBH₄ | sodium borohydride |
| | hexamethyldisilylamide | | |
| Et₃N or | Triethylamine | Py or Pyr | pyridine |
| TEA | | | |
| DMAP | 4-(dimethyl- | DIPEA | N,N- |
| | amino)pyridine | | diisopropylethylamine |
| CDI | 1,1′-Carbonyldiimidazole | NaNO₂ | Sodium nitrite |
| SnCl₂ | Stannous chloride | | |

### General Experimental

In the following examples, the chemical reagents were purchased from commercial sources (such as Alfa, Acros, Sigma Aldrich, TCI and Shanghai Chemical Reagent Company), and used without further purification. Flash chromatography was performed on an Ez Purifier III via column with silica gel particles of 200-300 esh. Analytical and preparative thin layer chromatography plates (TLC) were HSGF 254 (0.15-0.2 mm thickness, Shanghai Anbang Company, China). Nuclear magnetic resonance (NMR) spectra were recorded using Brucker AMX-300 or AMX-400 NMR (Brucker, Switzerland) at around 20-30° C. unless otherwise specified. The following abbreviations are used: s, singlet; d, doublet; t, triplet; q, quartet; m, multiplet; dd, doublet of doublets; ddd, doublet of doublet of doublet; dt, doublet of triplets; bs, broad signal. Chemical shifts were reported in parts per million (ppm, δ) downfield from tetramethylsilane. Mass spectra were run with electrospray ionization (ESI) from a Waters LCT TOF Mass Spectrometer (Waters, USA). Compound purification was carried out as needed using a variety of traditional methods including, but not limited to, preparative chromatography under acidic, neutral, or basic conditions using either normal phase or reverse phase HPLC or flash columns or Prep-TLC plates.

Preparative HPLC (Prep-HPLC): Unless otherwise described, the compounds were purified using a WATERS Fractionlynx system equipped with a YMC Pack Pro ds Column (5 μm, 120A, 50×20 mm) and the following solvent system: H₂O, MeCN, and 2% TFA in H₂O. Specific elution gradients were based on the retention times obtained with an analytical LC-MS, however, in general all elution gradients of H₂O and MeCN were run over a 7 minute run time with a flow rate of 35 mL/min. An autoblend method was used to ensure a concentration of 0.1% TFA throughout each run. Specific elution gradients were based on the retention times obtained with an analytical LC-MS, however, in general, all elution gradients of H₂O and MeCN were run over an 8 minute run time with a flow rate of 50 mL/min.

Analytical LC-MS: Analytical LC-MS was performed on a WATERS Acquity UPLC-MS instrument equipped with a ACQUITY UPLC BEH Ci₈ Column (2.1×50 mm, 1.7 μm), a column temperature of 45° C. and using the following solvent system: Solvent A: 0.1% HCOOH in H₂O; and Solvent B: 0.1% HCOOH in AcCN. All compounds were run using the same elution gradient, i.e., 5% to 95% Solvent B over a 1.5 min run time with a flow rate of 0.6 mL/min.

Examples 1-35 show the exemplified synthesis of the compounds as described herein.

### Example 1

### Synthesis of 5-hydrazinyl-3-(2-(pyridin-4-vl)propan-2-vl)-1,2,4-oxadiazole

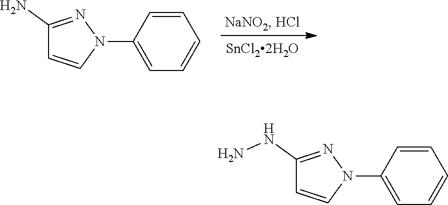 [SMILES: Nc1ccn(-c2ccccc2)n1 ; NNc1ccn(-c2ccccc2)n1]

To a solution of 1-phenyl-1H-pyrazol-3-amine (200 mg, 1.26 mmol) in HCl (2 M, 3 mL) was added NaNO₂ (174 mg, 2.52 mmol) dropwise over 5 min at 0° C. The mixture was stirred at 0° C.for 40 min and SnCl₂.2H₂O (853 mg, 3.78 mmol) was added dropwise. The reaction was stirred at 0° C.for 3 h. The mixture was concentrated under reduced pressure and the residue was purified by Pre-HPLC to afford 3-hydrazinyl-1-phenyl-1H-pyrazole (22 mg, 10% yield) as a white solid. ¹H NMR (400 MHZ, Methanol-d4) δ 8.03 (d, J=2.8 Hz, 1H), 7.67 (d, J=8.0 Hz, 2H), 7.44-7.40 (m, 2H), 7.23-7.19 (m, 1H), 6.01 (s, 1H); LCMS (M+H)⁺: 175.

### Example 2

### Synthesis of 3-hydrazinyl-5-(o-tolyl)-4H-1,2,4-triazole

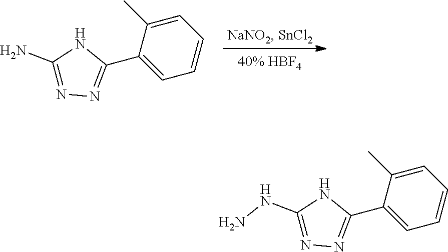

To a stirred solution of 5-(o-tolyl)-4H-1,2,4-triazol-3-amine (348 mg, 2.0 mmol) in HBF4 (5 ml, wt: 30% in water) was added a solution of NaNO₂ (276 mg, 4.0 mmol) in water (0.5 mL) dropwise at 0° C. The resulting mixture was stirred for 30 min before dropwise added a solution of SnCl₂.2H₂O (1.8 g, 8.0 mmol) in water (0.5 mL). The reaction mixture was allowed to warm up to at room temperature and stirred for 3 h. The mixture was concentrated under reduced pressure. The residue was purified by Prep-HPLC to give 3-hydrazinyl-5-(o-tolyl)-4H-1,2,4-triazole (30 mg, 4.0% yield). ¹H NMR (400 MHZ, CD3OD) δ: 7.55-7.52 (m, 1H), 7.34-7.21 (m, 3H), 2.45 (s, 3H); LCMS (M+1)⁺: 190.

### Example 3

### Synthesis of 3-cyclobutyl-5-hydrazinyl-4H-1,2,4-triazole

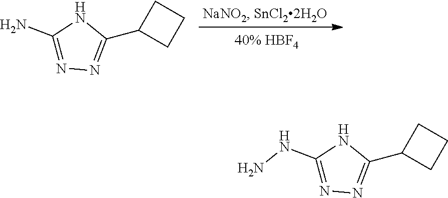 [SMILES: Nc1nnc(C2CCC2)[nH]1 ; NNc1nnc(C2CCC2)[nH]1]

To a solution of 5-cyclobutyl-4H-1,2,4-triazol-3-amine (200 mg, 1.45 mmol) in 40% HBF4 (3 ml) was added NaNO₂(188 mg, 2.72 mmol) dropwise over 5 min. at 0° C. The mixture was stirred at 0° C.for 40 min and SnCl₂.2H₂O (922 mg, 4.08 mmol) was added dropwise. The reaction was stirred at 0° C. for 3h. The mixture was concentrated under reduced pressure and the residue was purified by Prep-HPLC to afford 3-cyclobutyl-5-hydrazinyl-4H-1,2,4-triazole (4.2 mg, 2% yield) as white solid. ¹H NMR (400 MHZ, CD₃OD) δ 8.10 (s, 1H), 3.55-3.48 (m, 1H), 2.36-2.30 (m, 4H), 2.11-2.04 (m, 1H), 1.97-1.93 (m, 1H); LCMS (M+1)⁺: 154

### Example 4

### Synthesis of 3-hydrazinyl-5-propyl-4H-1,2,4-triazole

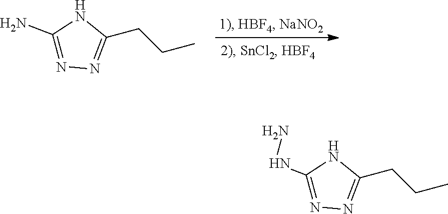

To a solution of 5-propyl-4H-1,2,4-triazol-3-amine (100 mg, 0.79 mmol) in HBF₄ (1 mL) at −10° C.was added NaNO₂ (55 mg, 0.79 mmol, in water (0.5 mL)) dropwise. Then the reaction mixture was added to a solution of SnCl₂ (451 mg, 2.38 mmol) in HBF₄ (2 mL) at −10° C. slowly and the reaction mixture was stirred and allowed to warm to room temperature over 60 minutes. The reaction mixture was concentrated and the residue was purified by Prep-HPLC to give 3-hydrazinyl-5-propyl-4H-1,2,4-triazole (12.1 mg, 10.8% yield) as a pale oil. ¹H NMR (400 MHZ, CD₃OD) δ 2.73 (t, J=7.6 Hz, 1H), 2.68 (t, J=7.6 Hz, 1H), 1.80-1.722 (m, 2H), 1.02-0.96 (m, 3H); LC-MS (M+1)⁺: 142.

### Example 5

### Synthesis of 4-(5-hydrazinyl-1H-1,2,4-triazol-3-yl)morpholine

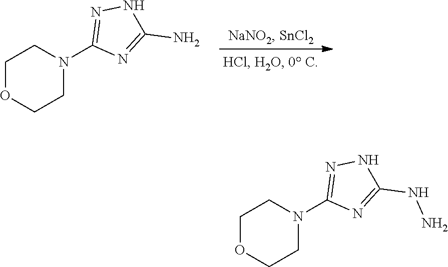 [SMILES: Nc1nc(N2CCOCC2)n[nH]1 ; NNc1nc(N2CCOCC2)n[nH]1]

To a solution of 3-morpholino-1H-1,2,4-triazol-5-amine (200 mg, 1.18 mmol) in HCl (2 M, 3 mL) was added NaNO₂ (163 mg, 2.36 mmol) dropwise over 5 min at 0° C. The mixture was stirred at 0° C.for 40 min and SnCl2.2H₂O (673 mg, 3.54 mmol) was added dropwise. The reaction was stirred at 0° C. for 3 h. The mixture was concentrated under reduced pressure and the residue was purified by Pre-HPLC to afford 4-(5-hydrazinyl-1H-1,2,4-triazol-3-yl)morpholine (18 mg, 8% yield) as a white solid. ¹H NMR (400 MHZ, CD₃OD) δ 3.70 (t, J=4.8 Hz, 4H), 3.39-3.33 (t, J=4.8 Hz, 4H); LC-MS (M+1)⁺: 185.

### Example 6

### Synthesis of 3-hydrazinyl-1-(2,2,2-trifluoroethyl)-1H-pyrazole

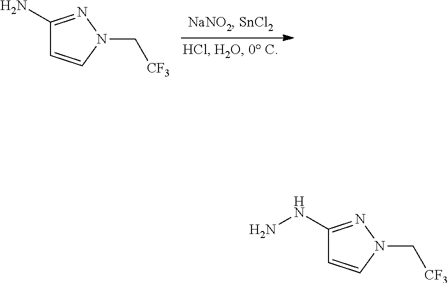 [SMILES: Nc1ccn(CC(F)(F)F)n1 ; NNc1ccn(CC(F)(F)F)n1]

To a solution of 1-(2,2,2-trifluoroethyl)-1H-pyrazol-3-amine (100 mg, 0.61 mmol) in HCl (2 M, 3 mL) was added NaNO₂ (84.2 mg, 1.22 mmol) dropwise over 5 min at 0° C. The mixture was stirred at 0° C.for 40 min and SnCl₂.2H₂O (346 mg, 1.83 mmol) was added dropwise. The reaction was stirred at 0° C. for 3 h. The mixture was concentrated under reduced pressure and the residue was purified by Pre-HPLC to afford 3-hydrazinyl-1-(2,2,2-trifluoroethyl)-1H-pyrazole (20 mg, 18% yield) as a white solid. ¹H NMR (400 MHZ, CD₃OD) δ 8.21 (s, 1H), 7.57 (d, J=2.8 Hz, 1H), 5.89 (d, J=2.4 Hz, 1H), 4.76 (q, J=8.8 Hz, 2H); LC-MS (M+1)⁺: 181.

### Example 7

### Synthesis of 3-hydrazinyl-1-isopropyl-1H-pyrazole

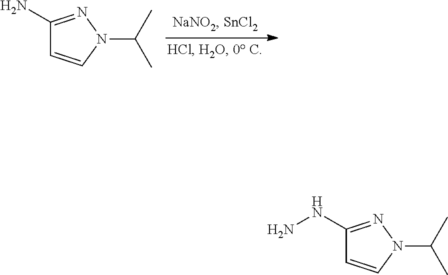

To a solution of 1-isopropyl-1H-pyrazol-3-amine (100 mg, 0.80 mmol) in HCl (2 M, 3 mL) was added NaNO₂ (110.4 mg, 1.60 mmol) dropwise over 5 min at 0° C. The mixture was stirred at 0° C.for 40 min and SnCl₂.2H₂O (456 mg, 2.40 mmol) was added dropwise. The reaction was stirred at 0° C.for 3 h. The mixture was concentrated under reduced pressure and the residue was purified by Pre-HPLC to afford 3-hydrazinyl-1-isopropyl-1H-pyrazole (33 mg, 28% yield) as a white solid. ¹H NMR (400 MHZ, CD₃OD) 8 7.50 (d, J=1.6 Hz, 1H), 5.80 (d, J=2.0 Hz, 1H), 4.42-4.33 (m, 1H), 1.44 (d, J=6.8 Hz, 6H); LC-MS (M+1)⁺: 141.

### Example 8

### Synthesis of 1-(2,2-difluoroethyl)-3-hydrazinyl-1H-pyrazole

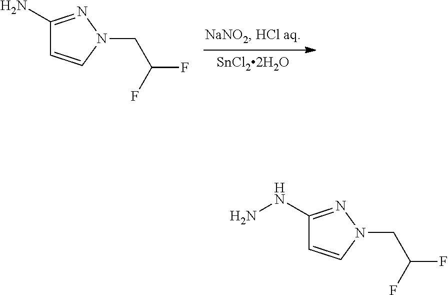 [SMILES: Nc1ccn(CC(F)F)n1 ; NNc1ccn(CC(F)F)n1]

To a solution of 1-(2,2-difluoroethyl)-1H-pyrazol-3-amine (200 mg, 1.36 mmol) in HCl (2 M, 3 mL) was added NaNO₂ (187.7 mg, 2.72 mmol) dropwise over 5 min at 0° C. The mixture was stirred at 0° C.for 40 min and SnCl₂.2H₂O (775 mg, 4.08 mmol) was added dropwise. The reaction was stirred at 0° C.for 3 h. The mixture was concentrated under reduced pressure and the residue was purified by Pre-HPLC to afford 1-(2,2-difluoroethyl)-3-hydrazinyl-1H-pyrazole (35 mg, 16% yield) as a white solid. ¹H NMR (400 MHZ, D20) δ 7.56 (d, J=2.8 Hz, 1H), 6.31-5.93 (m, 2H), 4.42 (td, J=15.2, 3.6 Hz, 2H); LC-MS (M+1)⁺: 163.

### Example 9

### Synthesis of 3-hydrazinyl-5-phenyl-4H-1,2,4-triazole

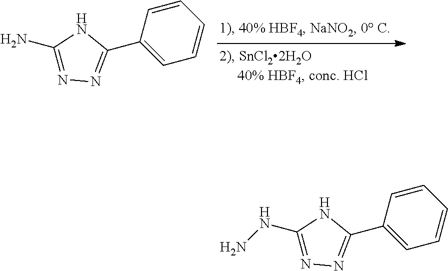 [SMILES: Nc1nnc(-c2ccccc2)[nH]1 ; NNc1nnc(-c2ccccc2)[nH]1]

To a stirred suspension of 5-phenyl-4H-1,2,4-triazol-3-amine (160 mg, 1 mmol) and water (0.3 mL) was added aq. tetrafluoroboric acid (3 mL, 48%) in small portions. After the final suspension had been cooled to 0° C., a concentrated aqueous sodium nitrite (69 mg, 1 mmol) was added dropwise, and the mixture was stirred for 10 min at 0° C. The white solid was collected by filtration which was used immediately in the next step. The solid was added in small portions to a stirred solution of stannous chloride (563 mg, 2.5 mmol) in concentrated hydrochloric acid (2 mL) and 48% tetrafluoroboric acid (2 mL) at 0° C. After the mixture had been stirred for 5 min, the solid was collected by filtration and further purified by prep-HPLC to give pure 3-hydrazinyl-5-phenyl-4H-1,2,4-triazole (50 mg, 28.6%) as a white solid. ¹H NMR (400 MHZ, CD₃OD) δ 8.03-7.81 (m, 2H), 7.57-7.51 (m, 3H); LC-MS (M+1)⁺: 176.1.

### Example 10

### Synthesis of 2-(3-hydrazinyl-1H-pyrazol-1-yl)acetic Acid

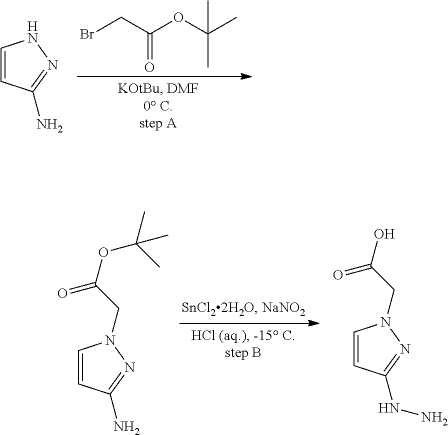

Step A. Synthesis of tert-butyl 2-(3-amino-1H-pyrazol-1-yl) acetate

At 0° C., tBuOK (1.9 g, 16.6 mmol) was added to a solution of 1H-pyrazol-3-amine (1.2 g, 14.5 mmol) in DMF (15 mL). The mixture was stired for 40 min before tert-butyl 2-bromoacetate (3.2 g, 16.6 mmol) in DMF (2 mL) was added. The reaction was stirred at 0° C. for 10 min and then warmed up to RT and stirred for 1 h. H₂O (50 mL) was added and extracted with EtOAc (50 mL×3). The organic phase was concentrated, the residue was puried by FC to give tert-butyl 2-(3-amino-1H-pyrazol-1-yl) acetate (1.1 g, 38.6% yield) as a white solid. LC-MS (M+1)⁺: 198.1.

Step B. Synthesis of 2-(3-hydrazinyl-1H-pyrazol-1-yl)acetic acid

To a solution of tert-butyl 2-(3-amino-1H-pyrazol-1-yl)acetate (1.1 g, 5.6 mmol) in conc.HCl (10 mL) and MeOH (10 mL) was added NaNO₂ (390 mg, 5.6 mmol) in H₂O (1 mL) at −5° C. The mixture was stired at −5° C.for 1 h before SnCl.2H₂O (3.8 g, 16.8 mmol) in conc.HCl (20 mL) was dropwise added. The reaction was stired for another 1 h at −5° C. The solvent was removed under reduced pressure. The residue was puried by Prep-HPLC to give 2-(3-hydrazinyl-1H-pyrazol-1-yl)acetic acid as a white solid (380 mg, 43.5% yield). ¹H NMR (400 MHZ, D₂O) δ 7.52 (d, J=2.5 Hz, 1H), 5.97 (d, J=2.5 Hz, 1H), 4.88 (s, 2H); LCMS (M+1)⁺: 157.

### Example 1

### Synthesis of 3-(1-methylhydrazinyl)-5-(trifluoromethyl)-4H-1,2,4-triazole

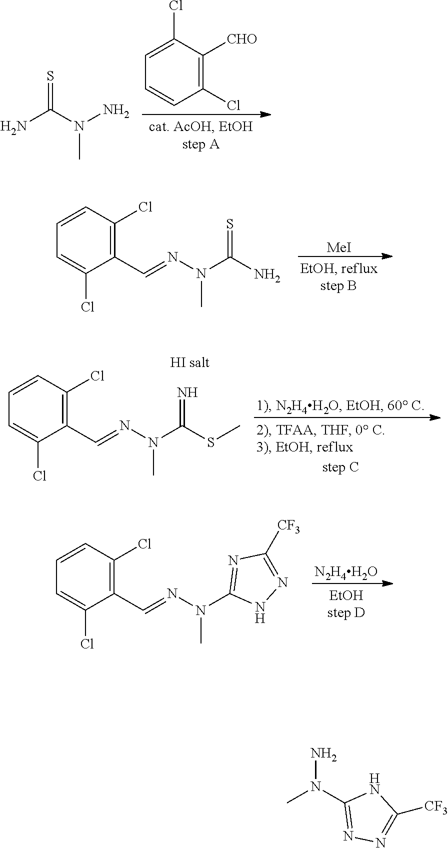

Step A. Synthesis of (E)-2-(2,6-dichlorobenzylidene)-1-methylhydrazine-1-carbothioamide

To a stirred solution of 2,6-dichlorobenzaldehyde (1.74 g, 9.98 mmol) in EtOH (20 mL) was added 1-methylhydrazine-1-carbothioamide (1.0 g, 9.51 mmol) and a drop of AcOH. The resulting mixture was heated to reflux and stirred for 6 hrs. After cooling to 0° C., the resulting precipitates were collected by filtration, washed with PE and dried in vacuo to give (E)-2-(2,6-dichlorobenzylidene)-1-methylhydrazine-1-carbothioamide (2.3 g, 93%yield) as a white solid. LCMS (M+1)⁺: 262.

Step B. Synthesis of methyl (E)-2-(2,6-dichlorobenzylidene)-1-methylhydrazine-1-carbimidothioate HI salt.

To a mixture of (E)-2-(2,6-dichlorobenzylidene)-1-methylhydrazine-1-carbothioamide (1.2 g, 4.57 mmol) in EtOH (30 mL) was added Mel (714 mg, 5.03 mmol). The resulting mixture was stirred at 65° C. under N₂ for 3 hr. After cooling to r.t, the reaction solution was concentrated in vacuo to dryness to give the desired product methyl (E)-2-(2,6-dichlorobenzylidene)-1-methylhydrazine-1-carbimidothioate HI salt as a white solid (1.8 g, 100% yield). LC-MS (M+1) : 276.0.

Step C. Synthesis of (E)-5-(2-(2,6-dichlorobenzylidene)-1-methylhydrazinyl)-3-(trifluoromethyl)-1H-1,2,4-triazole.

To a mixture of methyl (E)-2-(2,6-dichlorobenzylidene)-1-methylhydrazine-1-carbimidothioate HI salt (900 mg, 2.22 mmol) in EtOH (50 mL) was added N₂H₄.H₂O (334 mg, 6.68 mmol). The resulting mixture was stirred at 60° C. under N₂ for 3 hr till LCMS showed most of starting material was converted into hydrazine substituted intermediate. After cooling to r.t, the reaction solution was concentrated in vacuo to dryness and then dissolved in THF (50 mL). TFAA (1.2 g, 5.77 mmol) was added dropwise at 0° C. After the addition, the resulting mixture was stirred at r.t for 0.5 hr till LCMS showed the acylation was almost complete. Then, the mixture was concentrated in vacuo and the residue was diluted with EtOH (50 mL), followed by the addition of conc. H₂SO₄ (4 drops). The resulting solution was heated to reflux and stirred for 24 hr. After cooling to 0° C., the mixture was poured into ice water and extracted with EtOAc twice, the combined extracts were washed with brine, concentrated in vacuo and the residue was purified by column chromatography (Silica gel 60-120, 1:5, ethyl acetate/hexane) to give (E)-5-(2-(2,6-dichlorobenzylidene)-1-methylhydrazinyl)-3-(trifluoromethyl)-1H-1,2,4-triazole as a white solid (200 mg, 18.1% yield). LC-MS (M+1)⁺: 338.0.

Step D. Synthesis of 3-(1-methylhydrazinyl)-5-(trifluoromethyl)-4H-1,2,4-triazole.

To a mixture of (E)-5-(2-(2,6-dichlorobenzylidene)-1-methylhydrazinyl)-3-(trifluoromethyl)-1H-1,2,4-triazole (100 mg, 0.295 mmol) in EtOH (3 mL) was added N₂H_{4.}H₂O (0.1 mL). The resulting mixture was stirred at 100° C.in a sealed tube for 14 hr till LCMS showed the completion of the reaction. After concentration in vacuo, the residue was purified by prep-HPLC to give 3-(1-methylhydrazinyl)-5-(trifluoromethyl)-4H-1,2,4-triazole as a white solid (12 mg, 27% yield). ¹H NMR (400 MHZ, DMSO-d₆) δ 13.04 (s, 1H), 4.84 (s, 2H), 3.11 (s, 3H); LCMS (M+1)⁺: 182.1

### Example 12

### Synthesis of 4-(1-methylhydrazinyl)-5-(trifluoromethyl)-2H-1,2,3-triazole

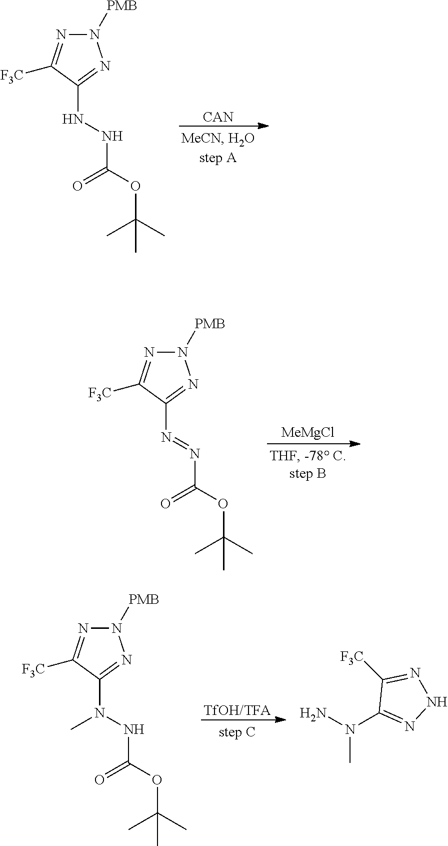

Step A. Synthesis of (E)-tert-butyl 2-(2-(4-methoxybenzyl)-5-(trifluoromethyl)-2H-1,2,3-triazol-4-yl)diazenecarboxylate.

To a solution of tert-butyl 2-(2-(4-methoxybenzyl)-5-(trifluoromethyl)-2H-1,2,3-triazol-4-yl)hydrazinecarboxylate (116 mg, 0.3 mmol) in MeCN (9 mL) was added dropwise a solution of CAN (550 mg, 1 mmol) at 0° C. After the addition, the solution was stirred at 0° C. for 0.5 h till TLC indicated the completion of the reaction. Then, the reaction solution was diluted with water and extracted with EtOAc twice. The combined extracts were washed with brine, dried over Na₂SO₄, concentrated in vacuo and purified by prep-TLC to give (E)-tert-butyl 2-(2-(4-methoxybenzyl)-5-(trifluoromethyl)-2H-1,2,3-triazol-4yldiazenecarboxylate (80 mg, 69.3%) as a yellow oil. LCMS (M+1)⁺: 386.1.

Step B. Synthesis of tert-butyl 2-(2-(4-methoxybenzyl)-5-(trifluoromethyl)-2H-1,2,3-triazol-4-yl)-2-methylhydrazinecarboxylate.

To a solution of (E)-tert-butyl 2-(2-(4-methoxybenzyl)-5-(trifluoromethyl)-2H-1,2,3-triazol-4-yl)diazenecarboxylate (77 mg, 0.2 mmol) in dry THF (10 mL) was added dropwise MeMgCl (1.0 mL, 1 mmol, 1.0 N in THF) at −78° C. over 3 minutes. After the addition, the mixture was stirred at −78° C. for 10 minutes before it was quenched by the addition of sat. NH₄Cl. The final mixture was partitioned between water and EtOAc. The organic layer was separated, washed with brine, concentrated in vacuo and purified by prep-TLC to give tert-butyl 2-(2-(4-methoxybenzyl)-5-(trifluoromethyl)-2H-1,2,3-triazol-4-yl)-2-methylhydrazinecarboxylate (50 mg, 62.5%) as a white solid. ¹H NMR (400 MHZ, DMSO-d₆) 8 9.38 (s, 1H), 7.34 (d, J=8.4 Hz, 2H), 6.99 (d, J=8.4 Hz, 2H), 5.50 (s, 2H), 3.79 (d, J=4.3 Hz, 3H), 3.08 (s, 3H), 1.43 (s, 9H); LCMS (M+1)⁺:402.2.

Step C. Synthesis of 4-(1-methylhydrazinyl)-5-(trifluoromethyl)-2H-1,2,3-triazole.

To a solution of tert-butyl 2-(2-(4-methoxybenzyl)-5-(trifluoromethyl)-2H-1,2,3-triazol-4-yl)-2-methylhydrazinecarboxylate (40 mg, 0.1 mmol) in TFA (0.8 mL) was added TfOH (0.2 mL), The resulting solution was stirred at 25° C. overnight. LCMS showed the reaction was complete. The solvents were removed by evaporation and the residue was directly purified by prep-HPLC to give 4-(1-methylhydrazinyl)-5-(trifluoromethyl)-2H-1,2,3-triazole (7 mg, 41.9%) as a white solid. ¹H NMR (600 MHZ, DMSO-d₆) δ 14.68 (s, 1H), 4.65 (s, 2H), 3.02 (s, 3H); LC-MS (M+1)⁺: 182.1.

### Example 13

### Synthesis of 4-hydrazinvl-5-(trifluoromethvl)-2H-1.2.3-triazole

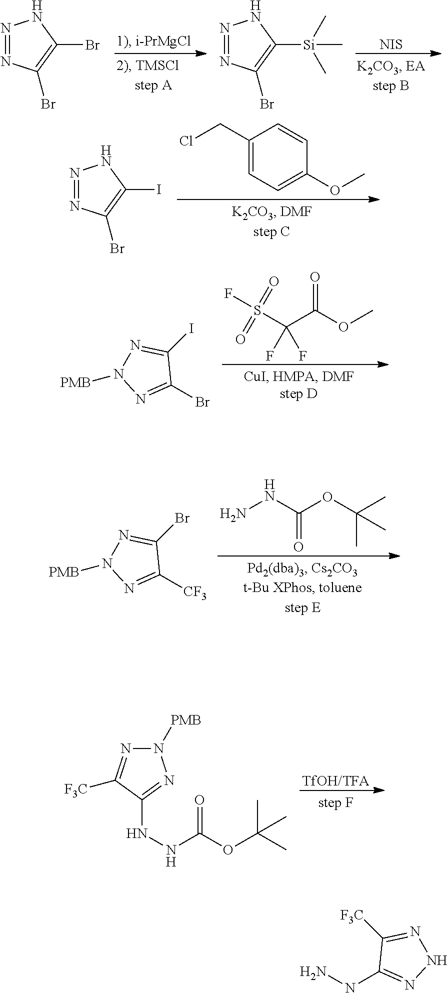

Step A. Synthesis of 4-bromo-5-(trimethylsilyl)-1H-1,2,3-triazole.

To a mixture of 4,5-dibromo-1H-1,2,3-triazole (2.27 g, 10 mmol) in dry THF (50 mL) was added i-PrMgCl (11 mL, 22 mmol, 2.0 N in THF) dropwise at −10° C. After the addition, the mixture was stirred at −5° C. for 1 h before TMSCI (2.16 g, 20 mmol) was added dropwise at −10 oC over 5 minutes. Then the reaction mixture was warmed to r.t. and stirred for 2 hours. After the reaction completed, the mixture was treated with sat. NH₄Cl, extracted with EA, dried over Na₂SO₄ and concentrated in vacuo. The residue was purified by flash chromatography (silica gel, 2% -10% EtOAc in PE) to give 4-bromo-5-(trimethylsilyl)-1H-1,2,3-triazole (1.3 g, 59.4%) as a yellow oil. LCMS (M+1)⁺: 220.

StepB. Synthesis of 4-bromo-5-iodo-1H-1,2,3-triazole

To a slurry of 4-bromo-5-trimethylsilyl-1,2,3-triazole 2 (1.3 g, 5.9 mmol) and K₂CO₃ (138 mg, 1 mmol) in EtOAc (20 mL) was added NIS (1.46 g, 6.5 mmol) at r.t. The mixture was allowed to react for 2 h at r.t. and quenched with 1% Na₂SO₃ (20 mL) and EtOAc (30 mL). The organic layer was washed with H₂O (30 mL) and dried over MgSO₄. The solvent in organic layer was evaporated under vacuum. The crude product was purified by flash chromatography (silica gel, 5% -20% EtOAc in PE) to give 4-bromo-5-iodo-1H-1,2,3-triazole (0.7 g, 43.7%) as a pale yellow solid. LCMS (M+1)⁺: 274.

Step C. Synthesis of 4-bromo-5-iodo-2-(4-methoxybenzyl)-2H-1,2,3-triazole

To a solution of 4-bromo-5-iodo-1H-1,2,3-triazole (0.7 g, 2.55 mmol) in dry DMF (15 mL) was added K2CO₃ (1.06 g, 7.66 mmol), follow by the addition of PMBCI (0.48 g, 3.06 mmol) at 0° C. After the addition, the mixture was stirred at 25° C. for 12 h until LCMS showed the reaction was complete. Then the reaction mixture was treated with ice-water and extracted with EA twice. The combined extracts were washed with brine, dried over Na₂SO₄ and concentrated in vacuo. The residue was purified by flash chromatography (silica gel, 5% -10% EtOAc in PE) to give 4-bromo-5-iodo-2-(4-methoxybenzyl)-2H-1,2,3-triazole (650 mg, 65%) as a pale yellow solid. ¹H NMR (400 MHZ, CDC₁₃) δ 7.24 (d, J=8.7 Hz, 2H), 6.81 (d, J=8.7 Hz, 2H), 5.40 (s, 2H), 3.73 (s, 3H); LCMS (M+1) : 394.

Step D. Synthesis of 4-bromo-2-(4-methoxybenzyl)-5-(trifluoromethyl)-2H-1,2,3-triazole

A mixture consisting of 4-bromo-5-iodo-2-(4-methoxybenzyl)-2H-1,2,3-triazole (197 mg, 0.5 mmol), methyl 2,2-difluoro-2-(fluorosulfonyl)acetate (288 mg, 1.5 mmol), CuI (95 mg, 0.5 mmol), HMPA (270 mg, 1.5 mmol) and DMF (6 mL) was stirred in a sealed tube under N₂ at 100° C. for 6 h. TLC showed ˜40% of desired product, ˜30% of starting material and ˜30% of byproduct with much more polarity. After cooling to r.t, the reaction mixture was treated with water and extracted with EA twice. The combined extracts were washed with brine, dried over Na₂SO₄ and concentrated in vacuo. The residue was purified by flash chromatography (silica gel, 2% -10% EA in PE) to give 4-bromo-2-(4-methoxybenzyl)-5-(trifluoromethyl)-2H-1,2,3-triazole (50 mg, 30%) as a white solid. ¹H NMR (400 MHZ, CDCl3) δ 7.24 (d, J=8.7 Hz, 2H), 6.81 (d, J=8.7 Hz, 2H), 5.41 (s, 2H), 3.73 (s, 3H); LCMS (M+1)⁺: 336.

Step E. Synthesis of tert-butyl 2-(2-(4-methoxybenzyl)-5-(trifluoromethyl)-2H-1,2,3-triazol-4-yl)hydrazinecarboxylate

A mixture consisting of 4-bromo-2-(4-methoxybenzyl)-5-(trifluoromethyl)-2H-1,2,3-triazole (34 mg, 0.1 mmol), tert-butyl hydrazinecarboxylate (16 mg, 0.12 mmol), Pd2(dba)3 (8 mg, 0.009 mmol), Di-tert-butyl(2′,4′,6′-triisopropylbiphenyl-2-yl)phosphine (6 mg, 0.014 mmol), Cs2CO3 (65 mg, 0.2 mmol) and dry toluene (3 mL) was stirred in a sealed tube under N2 at 100° C. for 4 h. LCMS showed the reaction was complete. After cooling to r.t, the reaction mixture was diluted with EA and filtered through celite. The filtrate was concentrated in vacuo and the residue was purified by preparative TLC to give tert-butyl 2-(2-(4-methoxybenzyl)-5-(trifluoromethyl)-2H-1,2,3-triazol-4-yl)hydrazinecarboxylate (26 mg, 67%) as a white solid. LCMS (M+1)⁺: 388.

Step F. Synthesis of 4-hydrazinyl-5-(trifluoromethyl)-2H-1,2,3-triazole

To a solution of tert-butyl 2-(2-(4-methoxybenzyl)-5-(trifluoromethyl)-2H-1,2,3-triazol-4-yl)hydrazinecarboxylate (25 mg, 0.064 mmol) in TFA (1.2 mL) was added TfOH (0.2 mL) at 0° C. After the addition, the mixture was stirred at 25° C. for 16 h and LCMS showed the reaction was complete. Then the reaction mixture was concentrated in vacuo to remove TFA and TfOH, the residue was co-evaporated with EtOH several times to remove residual TfOH as fully as possible. The final residue was purified by preparative HPLC to give 4-hydrazinyl-5-(trifluoromethyl)-2H-1,2,3-triazole (TfOH salt) (4 mg, 37.2%) as colorless oil. ¹H NMR (400 MHZ, DMSO-d₆) δ 7.20 (s, 1H), 7.07 (s, 1H), 6.95 (s, 1H); LCMS (M+1)⁺: 168.

### Example 14

### Synthesis of 2-hydrazinyl-5-methylthiazole

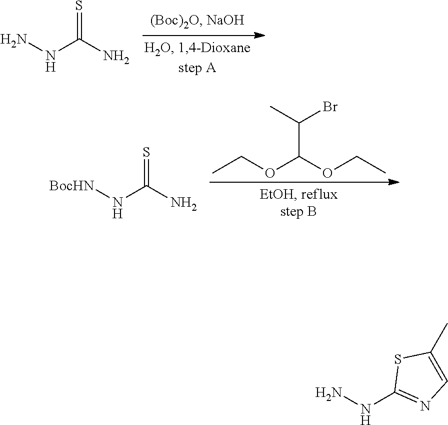

Step A. Synthesis of tert-butyl 2-carbamothioylhydrazine-1-carboxylate.

To a solution of hydrazinecarbothioamide (2.0 g, 21.96 mmol) in 1,4-dioxane (20 mL) and H₂O (20 mL) at 0° C.was added NaOH (877 mg, 21.94 mmol) and (Boc)₂O (5.75 g, 26.33 mmol). The reaction mixture was stirred at R.T for 2 hours. The reaction mixture was concentrated under reduced pressure, and purified by column chromatography (Silica gel 10-120, 1:1, ethyl acetate/hexane) to give tert-butyl 2-carbamothioylhydrazine-1-carboxylate as a white solid (1.2 g, 28% yield). LC-MS (M+1)⁺: 192.1.

Step B. Synthesis of 2-hydrazinyl-5-methylthiazole.

To a solution of tert-butyl 2-carbamothioylhydrazine-1-carboxylate (300 mg, 1.56 mmol) in EtOH (10 mL) and H₂O (10 mL), was added 2-bromo-1,1-diethoxypropane (500 mg, 2.34 mmol) and CH₃COOH(1drop). The reaction mixture was stirred at 100° C. overnight. The reaction mixture was concentrated under reduced pressure, and purified by column chromatography (dichloromethane/methanol=15:1) followed by Prep-HPLC to give 2-hydrazinyl-5-methylthiazole (4 mg, 2% yield) as a white solid. ¹H NMR (400 MHZ, DMSO-d₆) δ 6.97 (d, J=1.2 Hz, 1H), 2.25 (d, J=1.2 Hz, 3H); LCMS (M+1)⁺: 130.0.

### Example 15

### Synthesis of 2-hydrazinyl-5-(methoxymethyl)thiazole

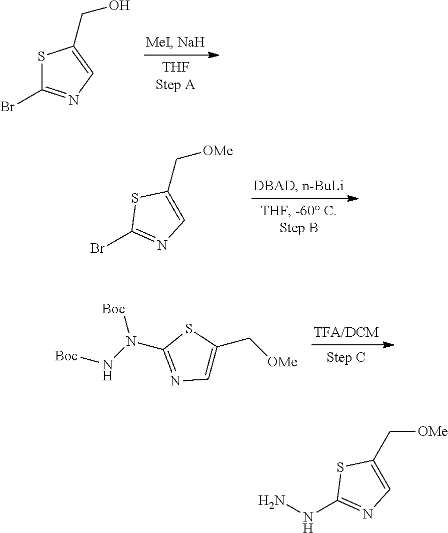 [SMILES: OCc1cnc(Br)s1 ; COCc1cnc(Br)s1 ; COCc1cnc(N(NC(=O)OC(C)(C)C)C(=O)OC(C)(C)C)s1 ; COCc1cnc(NN)s1]

Step A. Synthesis of 2-bromo-5-(methoxymethyl)thiazole.

To a suspension of NaH (412 mg, 10.3 mmol, 60% in oil) in anhydrous THF (10 mL) was added a solution of (2-bromothiazol-5-yl)methanol (1.0 g, 5.15 mmol) in THF dropwise at 0° C.and stirred at room temperature for 30 min. Then Mel (1 g, 7.73 mmol) was added to the reaction mixture at 0° C. The resulting mixture was stirred for 30 min at room temperature. After completion of the reaction, the mixture was poured into ice water and extracted with EtOAc (2×50 mL). The combined organic layers were washed with brine (1×50 mL), dried over Na₂SO₄, concentrated under reduced pressure and purified by column chromatography (Silica gel 100-200, 1:10, ethyl acetate/hexane) to give 2-bromo-5-(methoxymethyl)thiazole as a colorless oil (500 mg, 50% yield). LC-MS (M+1)⁺: 208.

Step B. Synthesis of di-tert-butyl 1-(5-(methoxymethyl)thiazol-2-yl)hydrazine-1,2-dicarboxylate.

To a solution of 2-bromo-5-(methoxymethyl)thiazole (500 mg, 2.4 mmol) in anhydrous THF (20 mL) was added n-BuLi (1.5 mL, 2.5 M in THF) dropwise at −60° C. under N₂ and stirred at this temperature for 20 min. Then a solution of DBAD (111 mg, 4.8 mmol) in THF was added dropwise at −60° C. The resulting mixture was stirred for 30 min at room temperature. After completion of the reaction, the mixture was quenched with aqueous NH₄Cl and extracted with EtOAc (2×60 mL). The combined organic layers were washed with brine (1×50 mL), dried over Na₂SO₄, concentrated under reduced pressure and purified by column chromatography (Silica gel 100-200, 1:5, ethyl acetate/hexane) to give the desired product di-tert-butyl 1-(5-(methoxymethyl)thiazol-2-yl)hydrazine-1,2-dicarboxylate as a colorless oil (500 mg, 34.7% yield). LC-MS (M+1)⁺: 360.

Step C. Synthesis of 2-hydrazinyl-5-(methoxymethyl)thiazole.

To a solution of di-tert-butyl 1-(5-(methoxymethyl)thiazol-2-yl)hydrazine-1,2-dicarboxylate (100 mg, 0.28 mmol) in DCM (2 mL) was added TFA (1 mL). The reaction mixture was stirred at room temperature for 2 h. After completion of the reaction, the mixture was concentrated under reduced pressure to furnish the desired compound 2-hydrazinyl-5-(methoxymethyl)thiazole as a yellow solid (30 mg, 72.8% yield). ¹H NMR (400 MHZ, CD₃OD) δ: 6.97 (s, 1H), 4.45 (s, 2H), 3.34 (s, 3H); LC-MS (M+1)⁺: 160.

### Example 16

### Synthesis of 3-hydrazinyl-5-(trifluoromethyl)-4H-1,2,4-triazole

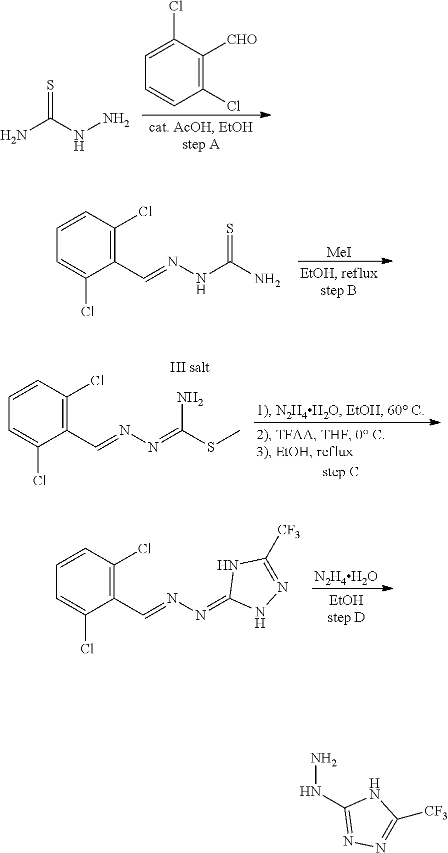

Step A. Synthesis of (E)-2-(2,6-dichlorobenzylidene)hydrazinecarbothioamide.

To a stirred solution of 2,6-dichlorobenzaldehyde (1.74 g, 10 mmol) in EtOH (20 mL) was added hydrazinecarbothioamide (0.91 g, 10 mmol) and a drop of AcOH. The resulting mixture was heated to reflux and stirred for 6 hr. After cooling to 0° C., the resulting precipitates were collected by filtration, washed with PE and dried in vacuo to give the desired compound, (E)-2-(2,6-dichlorobenzylidene)hydrazinecarbothioamide, as a gray solid (2.3 g, 93%yield). LCMS (M+1)⁺: 248.

Step B. Synthesis of (1E,N′E)-methyl N′-2,6-dichlorobenzylidenecarbamohydrazonothioate HI salt.

To a mixture of (E)-2-(2,6-dichlorobenzylidene)hydrazinecarbothioamide (1.8 g, 7.3 mmol) in EtOH (30 mL) was added Mel (1.14 g, 8 mmol). The resulting mixture was stirred at 65° C. under N₂ for 3 hr. The progress of the reaction was monitored by LCMS. After cooling to r.t, the reaction solution was concentrated in vacuo to dryness to give (1E,N′E)-methyl N′-2,6-dichlorobenzylidenecarbamohydrazonothioate HI salt as a yellow solid (2.85 g, 100% yield). LC-MS (M+1)⁺: 262.0.

Step C. Synthesis of (E)-5-((E)-(2,6-dichlorobenzylidene)hydrazono)-3-(trifluoromethyl)-4,5-dihydro-1H-1,2,4-triazole.

To a mixture of (1E,N′E)-methyl N′-2,6-dichlorobenzylidenecarbamohydrazonothioate HI salt (390 mg, 1.0 mmol) in EtOH (10 mL) was added N₂H₄.H₂O (150 mg, 3 mmol). The resulting mixture was stirred at 60° C. under N₂ for 3 hr till LCMS showed most of starting material was converted into hydrazine substituted intermediate. After cooling to r.t, the reaction solution was concentrated in vacuo to dryness and then dissolved in THF (10 mL). TFAA (630 mg, 3 mmol) was added dropwise at 0° C. After the addition, the resulting mixture was stirred at r.t for 0.5 hr till LCMS showed the acylation was almost complete. Then, the mixture was concentrated in vacuo and the residue was diluted with EtOH (10 mL), followed by the addition of conc. H₂SO₄ (0.5 mL). The resulting solution was heated to reflux and stirred for 24 hr. After cooling to 0° C., the mixture was poured into ice water and extracted with EtOAc twice, the combined extracts were washed with brine, concentrated in vacuo and the residue was purified by column chromatography (Silica gel 60-120, 1:5, ethyl acetate/hexane) to give (E)-5-((E)-(2,6-dichlorobenzylidene)hydrazono)-3-(trifluoromethyl)-4,5-dihydro-1H-1,2,4-triazole as a white solid (60 mg, 18.6% yield). LC-MS (M+1)⁺: 324.0.

Step D. Synthesis of 3-hydrazinyl-5-(trifluoromethyl)-4H-1,2,4-triazole.

To a mixture of (E)-5-((E)-(2,6-dichlorobenzylidene)hydrazono)-3-(trifluoromethyl)-4,5-dihydro-1H-1,2,4-triazole (50 mg, 0.15 mmol) in EtOH (3 mL) was added N₂H₄.H₂O (46 mg, 0.9 mmol). The resulting mixture was stirred at 100° C. in a sealed tube for 14 hr till LCMS showed the completion of the reaction. After concentration in vacuo, the residue was purified by prep-HPLC to give the desired product 3-hydrazinyl-5-(trifluoromethyl)-4H-1,2,4-triazole as a pale yellow solid (6 mg, 24.1% yield). ¹H NMR (400 MHz, DMSO-d₆) δ: 8.11 (d, J=4.6 Hz, 2H), 4.52 (br, 2H); LC-MS (M+1)⁺: 168.0.

### Example 17

### Synthesis of 3-(1-methylhydrazinyl)-1,2,4-oxadiazol-5(4H)-one

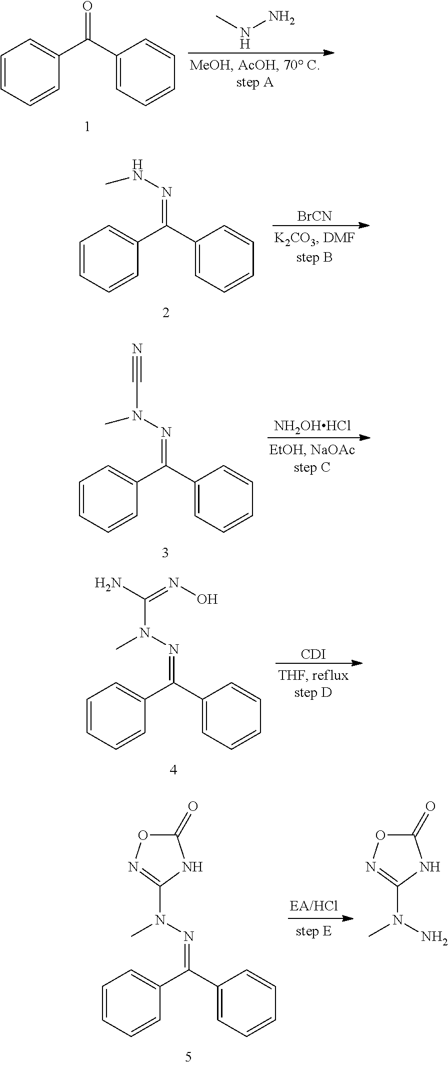

Step A. Synthesis of 1-(diphenylmethylene)-2-methylhydrazine

Methylhydrazine (17.4 mL, 0.33 mol) and glacial acetic acid (30 mL) were added to a solution of benzophenone (54.6 g, 0.30 mol) in methanol (100 mL). The mixture was heated at reflux for 2 h under a nitrogen atmosphere and then allowed to warm to room temperature. Half of the solvent was removed in vacuo. A saturated solution of NaHCO₃ was added and the mixture was extracted twice with EtOAc. The organic layer was dried with magnesium sulfate andconcentrated in vacuo. Addition of a minimum amount of methanol and storage in the freezer gave, after a few days, light yellow crystals which were collected by filtration, washed with methanol and dried in vacuo to give 1-(diphenylmethylene)-2-methylhydrazine as a white solid (20.1 g, 32% yield). LCMS (M+1)⁺: 211.

Step B. Synthesis of 2-(diphenylmethylene)-1-methylhydrazine-1-carbonitrile

To a mixture of 1-(diphenylmethylene)-2-methylhydrazine (0.9 g, 4.29 mmol) and K₂CO₃ (888 mg, 6.44 mmol) in DMF (10 mL) was added BrCN (683 mg, 6.44 mmol) in DCM at 0° C. Then the mixture was stirred at room temperature for 30 mins. The reaction mixture was diluted with water (50 mL) and extracted with DCM (2×50 mL). The combined organic layers were washed with brine (2×50 mL), dried over Na₂SO₄, concentrated under reduced pressure and purified by column chromatography (Silica gel 100-200, 1:4, ethyl acetate/hexane) to give 2-(diphenylmethylene)-1-methylhydrazine-1-carbonitrile as a colorless oil (750 mg, 74% yield). LCMS (M+1)⁺: 236.

Step C. Synthesis of (Z)-2-(diphenylmethylene)-N′-hydroxy-1-methylhydrazine-1-carboximidamide

To a mixture of 2-(diphenylmethylene)-1-methylhydrazine-1-carbonitrile (150 mg, 0.64 mmol) and hydroxylamine hydrochloride (66 mg, 0.96 mmol) in EtOH (3 ml) was added AcONa (78.7 mg, 0.96 mmol) at RT. Then the mixture was stirred at rt overnight. After completion of the reaction, the reaction mixture was extracted with EtOAc (2×50 mL). The combined organic layers were washed with brine (30 mL), died over anhydrous Na₂SO₄, filtered and concentrated under reduced pressure to give (Z)-2-(diphenylmethylene)-N′-hydroxy-1-methylhydrazine-1-carboximidamide (130 mg, 71% yield) as a white solid which was used in the next step without further purification. LCMS (M+1)⁺: 269.

Step D. Synthesis of 3-(2-(diphenylmethylene)-1-methylhydrazinyl)-1,2,4-oxadiazol-5(4H)-one

To a solution of (Z)-2-(diphenylmethylene)-N′-hydroxy-1-methylhydrazine-1-carboximidamide (130 mg, 0.49 mmol) in THF (3 ml) was added CDI (118 mg, 0.73 mmol). Then the mixture was stirred at 70° C.for 5 h. The reaction was concentrated under reduced pressure and purified by FCC to give 3-(2-(diphenylmethylene)-1-methylhydrazinyl)-1,2,4-oxadiazol-5(4H)-one (45 mg, 28% yield) as a white solid. LCMS (M+1)⁺: 295.

Step E. Synthesis of 3-(1-methylhydrazinyl)-1,2,4-oxadiazol-5(4H)-one

A solution of 3-(2-(diphenylmethylene)-1-methylhydrazinyl)-1,2,4-oxadiazol-5(4H)-one (45 mg, 0.15 mmol) in 2 M HCl in EtOAc (10 ml) was stirred at rt overnight. The reaction was concentrated under reduced pressure and purified by Prep-HPLC to give 3-(1-methylhydrazinyl)-1,2,4-oxadiazol-5(4H)-one (10 mg, 50% yield) as a white solid. ¹H NMR (400 MHZ, DMSO-d₆) § 2.95 (s, 3H); LC-MS (M+1)⁺: 131.1.

### Example 18

### Synthesis of 2-hydrazinyl-1,3,4-oxadiazole

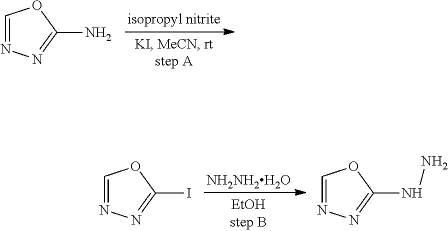

Step A. Synthesis of 2-iodo-1,3,4-oxadiazole.

To a solution of 1,3,4-oxadiazol-2-amine (200 mg, 2.35 mmol) in MeCN (5 mL) was added isopropyl nitrite (419 mg, 4.70 mmol) and KI (781 mg, 4.70 mmol) at 0° C. The reaction solution was stirred at room temperature for 2 hrs. Then the reaction solution was concentrated and the reisdue was purified by column chromatography to afford 2-iodo-1,3,4-oxadiazole (150 mg, 33%yield) as a yellow oil. LC-MS (M+1)⁺: 196.1

Step B. Synthesis of 2-hydrazinyl-1,3,4-oxadiazole.

To a solution of 2-iodo-1,3,4-oxadiazole (30 mg, 0.16 mmol) in EtOH (3 mL) was added hydrazine hydrate (0.2 mL, 98%). The reaction solution was stirred at room temperature for 2 hrs. Then the reaction solution was concentrated to dryness and the reisdue was purified by Prep-HPLC to afford 2-hydrazinyl-1,3,4-oxadiazole (10 mg, 27% yield) as a white solid. ¹H NMR (400 MHZ, DMSO-d₆) δ 8.54 (s, 1H), 8.47 (s, 1H), 4.42 (s, 2H); LCMS (M+1): 101.

### Example 19

### Synthesis of 2-hydrazinvlthiazole-5-carbonitrile

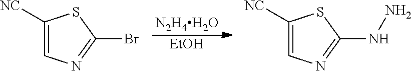

To a stirred solution of 2-bromothiazole-5-carbonitrile (50 mg, 0.27 mmol) in EtOH (5 mL) was added hydrazine hydrate (27 mg, 0.53 mmol) and stirred at 50° C.for 2 hrs. After completion of the reaction, the resulting mixture was concentrated and purified by Preparative HPLC to afford the desired compound, 2-hydrazinylthiazole-5-carbonitrile, as a solid (10 mg, 27% yield). ¹H NMR (400 MHZ, DMSO-d₆) δ 9.78 (s, 1H), 7.88 (s, 1H), 5.34 (s, 2H); LCMS (M+1): 141.

### Example 20

### Synthesis of 2-hydrazinylthiazole-5-carboxylic Acid

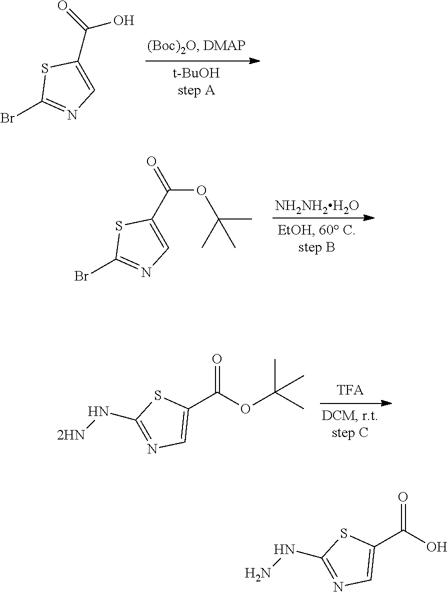

Step A. Synthesis of tert-butyl 2-bromothiazole-5-carboxylate.

To a stirred solution of 2-bromothiazole-5-carboxylic acid (200 mg, 1.0 mmol) in t-BuOH (5 mL) was added (Boc)20 (220 mg, 1.0 mmol), followed by DMAP (47 mg , 0.39 mmol) at room temperature. The reaction mixture was stirred at 50° C. overnight. The reaction mixture was partitioned between EtOAc (30 mL) and water (30 mL). The organic phase was washed with brine (20 mL), dried over Na₂SO₄, concentrated under reduced pressure and purified by column chromatography (Silica gel 100-200, 1:100, ethyl acetate/hexane) to give the desired product tert-butyl 2-bromothiazole-5-carboxylate as a yellow oil (100 mg, 38.1% yield). LC-MS (M+1)⁺: 264.

Step B. Synthesis of tert-butyl 2-hydrazinylthiazole-5-carboxylate.

To a stirred solution of tert-butyl 2-bromothiazole-5-carboxylate (50 mg, 0.17 mmol) in EtOH (3 mL) was added Hydrazinium hydroxide (34.1 mg , 0.68 mmol). The reaction mixture was stirred at 60° C.overnight. The reaction mixture was partitioned between EtOAc (10 mL) and water (10 mL). The organic phase was washed with brine (10 mL), dried over Na₂SO₄, concentrated under reduced pressure and purified by column chromatography (Silica gel 100-200, 1:1, ethyl acetate/hexane) to give the desired product tert-butyl 2-hydrazinylthiazole-5-carboxylate as a white solid (15 mg, 40.8% yield). LC-MS (M+1)⁺: 216.

Step C. Synthesis of 2-hydrazinylthiazole-5-carboxylic Acid.

To a stirred solution of tert-butyl 2-hydrazinylthiazole-5-carboxylate (15 mg, 0.07 mmol) in DCM (3 mL) was added TFA (1 mL). The reaction mixture was stirred at room temperature overnight and concentrated under reduced pressure to give the desired compound 2-hydrazinylthiazole-5-carboxylic acid (9 mg, 82.0% yield) as yellow solid. ¹H NMR (400 MHz, DMSO-d₆) δ: 9.31 (brs, 1H), 7.67 (s, 1H), 5.17 (brs, 1H); LC-MS (M+1)⁺: 160.

### Example 21

### Synthesis of 5-hydrazinyl-4H-1,2,4-triazol-3-ol

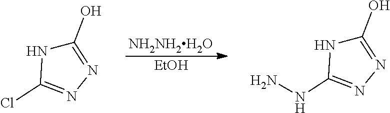

To a stirred solution of 5-chloro-4H-1,2,4-triazol-3-ol (20 mg, 0.17 mmol) in EtOH (1 mL) was added Hydrazine hydrate (1 mL). The reaction mixture was stirred at 80° C. for 3h and concentrated under reduced pressure to give the desired compound 5-hydrazinyl-4H-1,2,4-triazol-3-ol (8.7 mg, 45.0% yield) as yellow solid. ¹H NMR (400 MHZ, DMSO-d₆) δ 10.27 (s, 1H), 6.87 (m, 1H), 4.03 (brs, 2H); LC-MS (M+1)⁺: 116.

### Example 22

### Synthesis of 5-hydrazinyl-2-methyloxazole-4-carbonitrile

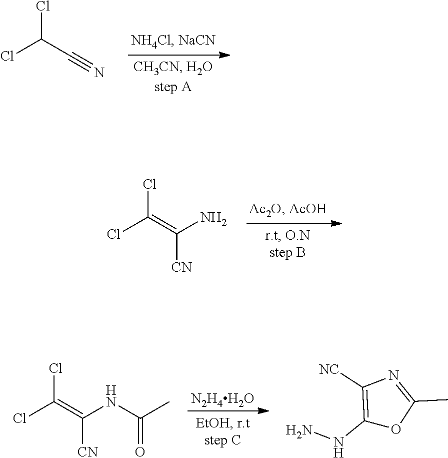

Step A. Synthesis of 2-amino-3,3-dichloroacrylonitrile.

To a stirred solution of 2,2-dichloroacetonitrile (500 mg, 4.59 mmol) in CH₃CN (10 mL), water (2 mL) was added NH₄Cl (486 mg, 9.17 mmol) and NaCN (450 mg, 9.17 mmol) and stirred at room temperature for 16 h. After completion of the reaction, the mixture was concentrated and washed with water to afford 2-amino-3,3-dichloroacrylonitrile (560 mg, 80%yield) as a white solid. LCMS (M+1): 137.

Step B. Synthesis of N-(2,2-dichloro-1-cyanovinyl)acetamide.

To a stirred solution of 2-amino-3,3-dichloroacrylonitrile (560 mg, 4.12 mmol) in HOAc (5 mL) was added Acetic anhydride (504 mg, 4.94 mmol) at room temperature and stirred for 16 h. After completion of the reaction, the mixture was quenched with NaHCO₃ solution and extracted with EtOAc (2×50 mL). The combined organic layers were concentrated under reduced pressure and purified by chromatography column to afford N-(2,2-dichloro-1-cyanovinyl)acetamide (500 mg, 68.2%yield) as a white solid. LCMS (M+1): 179.

Step C. Synthesis of 5-hydrazinyl-2-methyloxazole-4-carbonitrile.

To a stirred solution of N-(2,2-dichloro-1-cyanovinyl)acetamide (500 mg, 2.81 mmol) in EtOH (5 mL) was added hydrazine hydrate (281 mg, 5.62 mmol) at room temperature and stirred for 4 h. After completion of the reaction, the mixture was concentrated and purified by preparative HPLC to furnish the desired compound, 5-hydrazinyl-2-methyloxazole-4-carbonitrile (100 mg, 26% yield) as a white solid. ¹H NMR (400 MHZ, DMSO-d₆) δ 8.88 (brs, 1H), 4.72 (brs, 2H), 2.22(s, 3H). LCMS (M+1): 138.

### Example 23

### Synthesis of (2-hydrazinylthiazol-4-yl)methanol

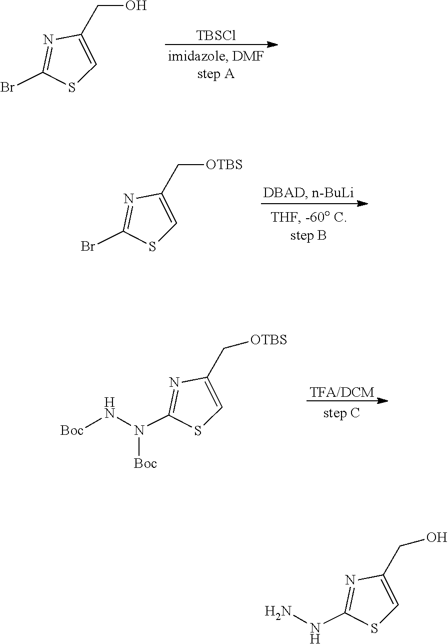

Step A. Synthesis of 2-bromo-4-(((tert-butyldimethylsilyl)oxy)methyl)thiazole.

To a stirred solution of (2-bromothiazol-4-yl)methanol (500 mg, 2.59 mmol) in DMF (10 mL) was added imidazole (530 mg, 7.77 mmol), followed by TBSCl (590 mg, 3.89 mmol) at room temperature. Then the reaction mixture was stirred at room temperature for 30 min. The reaction mixture was diluted with water (30 mL) and extracted with ethyl acetate (2×40 mL). The combined organic layers were washed with brine (2×40 mL), dried over Na₂SO₄, concentrated under reduced pressure and purified by column chromatography (Silica gel 100-200, 100:1, ethyl acetate/hexane) to give the desired product 2-bromo-4-(((tert-butyldimethylsilyl)oxy)methyl)thiazole as a colorless oil (400 mg, 50% yield). LCMS (M+1): 308.

Step B. Synthesis of di-tert-butyl 1-(4-(((tert-butyldimethylsilyl)oxy)methyl)thiazol-2-yl)hydrazine-1,2-dicarboxylate.

To a solution of 2-bromo-4-(((tert-butyldimethylsilyl)oxy)methyl)thiazole (200 mg, 0.65 mmol) in anhydrous THF (10 mL) was added n-BuLi (0.3 mL, 0.71 mmol, 2.5 M in THF) dropwise under N₂ atmosphere at −60° C. Then the mixture was stirred at this temperature for 30 mins. A solution of DBAD (230 mg, 0.98 mmol) in THF was added dropwise at −60° C. The resulting mixture was stirred at −60° C.for 30 mins. The mixture was quenched with aqueous NH₄Cl and extracted with ethyl acetate (2×50 mL). The combined organic layers were washed with brine (1×40 mL), dried over Na₂SO₄, concentrated under reduced pressure and purified by column chromatography (Silica gel 100-200, 3:1, ethyl acetate/hexane) to give the desired product di-tert-butyl 1-(4-(((tert-butyldimethylsilyl)oxy)methyl)thiazol-2-yl)hydrazine-1,2-dicarboxylate as a yellow oil (150 mg, 50% yield). LCMS (M+1): 460.

Step B. Synthesis of (2-hydrazinylthiazol-4-yl)methanol.

To a solution of di-tert-butyl 1-(4-(((tert-butyldimethylsilyl)oxy)methyl)thiazol-2-yl)hydrazine-1,2-dicarboxylate (70 mg, 0.15 mmol) in DCM (2 mL) was added TFA (0.5 mL). The mixture was stirred at room temperature for 16 hrs and concentrated under reduced pressure. The residue was purified by prep-HPLC to give desired product (2-hydrazinylthiazol-4-yl)methanol as a yellow oil (4 mg, 19.1% yield). ¹H NMR (400 MHz,CD₃OD) δ: 6.52 (s, 1H), 4.46 (s, 2H); LCMS (M+1): 146.

### Example 24

### Synthesis of 2-hydrazinyl-4-methylthiazole-5-carboxylic Acid

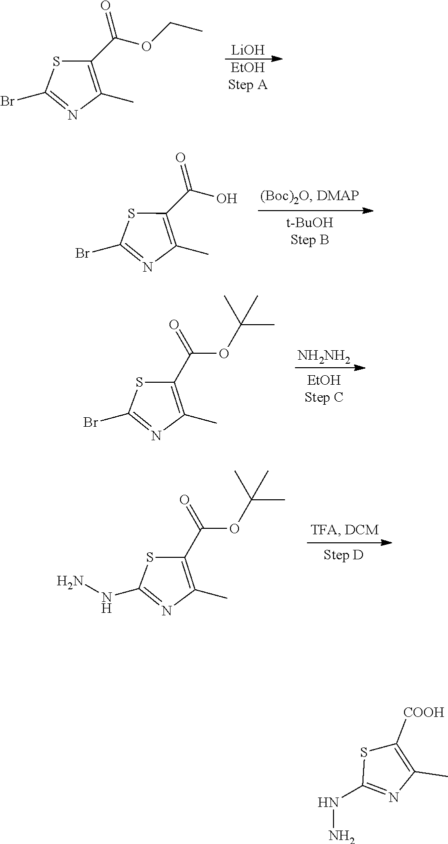

Step A. Synthesis of 2-bromo-4-methylthiazole-5-carboxylic acid.

To a solution of ethyl 2-bromo-4-methylthiazole-5-carboxylate (500 mg, 2 mmol) in EtOH (10 mL) and water (1 mL) was added LiOH (479 mg, 20 mmol), The resulting mixture was stirred at room temperature for 3 hrs. The mixture was neutralized with 1 N HCl, extracted with EtOAc (60 mL). The organic phase was washed with brine (20 mL×2), dried over Na₂SO₄, filtered, concentrated to afford 2-bromo-4-methylthiazole-5-carboxylic acid (320 mg, 69% yield) as a white solid. LCMS (M+1): 223.

Step B. Synthesis of tert-butyl 2-bromo-4-methylthiazole-5-carboxylate.

To a solution of 2-bromo-4-methylthiazole-5-carboxylic acid (80 mg 0.36 mmol) in t-BuOH (10 mL) was added (Boc)₂O (157 mg 0.72 mmol) and DMAP(13 mg, 0.108 mmol). The reaction mixture was stirred at 30° C. for 3 hrs. The reaction mixture was partitioned between EtOAc (50 mL) and water (15 mL). The organic layer was washed with brine (20 mL), dried over Na₂SO₄, filtered, concentrated to dryness and the residue was purified by by flash column chromatography on silica gel (PE/EA=12:1) to afford tert-butyl 2-bromo-4-methylthiazole-5-carboxylate (40mg, 39.7% yield) as a white solid. LCMS (M+1): 279.

Step C. Synthesis of tert-butyl 2-hydrazinyl-4-methylthiazole-5-carboxylate.

To a solution of Tert-butyl 2-bromo-4-methylthiazole-5-carboxylate (40 mg, 0.143mmol) in EtOH (3 mL) was added NH₂NH₂ H₂O (0.1 mL). The resulting mixture and stirred at 70° C. for 16 hrs. The reaction mixture was evaporated to dryness and the residue was purified by flash column chromatography on silica gel (PE/EA=3:1) to afford tert-butyl 2-hydrazinyl-4-methylthiazole-5-carboxylate (20 mg, 61% yield) as a white solid. LCMS (M+1): 230.

Step D. Synthesis of 4-((1-(aminooxy)cyclobutane-1-carbonyl)oxy)butanoicacid.

To a solution of tert-butyl 2-hydrazinyl-4-methylthiazole-5-carboxylate (20 mg, 0.087 mmol) in DCM (2 mL) was added TFA (2 mL). The resulting mixture was stirred at room temperature for 2 h. The reaction solution was then evaporated to afford to 4-((1-(aminooxy)cyclobutane-1-carbonyl)oxy)butanoicacid (15 mg, 99%yield) as a white solid. ¹H NMR (400 MHZ, DMSO-d₆) δ 10.16-9.61 (m, 1H), 2.41 (s, 3H); LCMS (M+1): 174.

### Example 25

### Synthesis of 5-hydrazinyl-1-methyl-1H-tetrazole

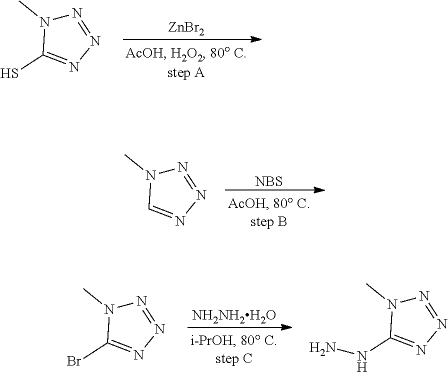

Step A. Synthesis of 1-methyl-1H-tetrazole.

To a stirred solution of 1-methyl-1H-tetrazole-5-thiol (500 mg, 4.3 mmol) in AcOH (10 mL) was added ZnBr2 (1.9 g, 8.6 mmol) at 40° C. Then H₂O₂ (975 mg, 8.6 mmol, wt=30%) was added to the mixture dropwise. The reaction mixture was stirred at 80° C. overnight. Then the reaction mixture was quenched with water (30 mL) and extracted with ethyl acetate (2×40 mL). The combined organic layers were washed with brine (1×40 mL), dried over Na₂SO₄, concentrated under reduced pressure to give the desired compound 1-methyl-1H-tetrazole (50 mg, 13.8% yield) as a white solid which was used in the next step without further purification. ¹H NMR (400 MHZ, DMSO-d₆) δ: 9.48 (s, 1H), 4.21 (s, 3H); LC-MS (M+1)⁺: 85.

Step B. Synthesis of 5-bromo-1-methyl-1H-tetrazole.

To a stirred solution of 1-methyl-1H-tetrazole (50 mg, 0.60 mmol) in AcOH (5 mL) was added NBS (127 mg, 0.71 mmol). The reaction mixture was stirred at 80° C. overnight and concentrated under reduced pressure. The residue was purified by column chromatography (Silica gel 100-200, 1:3, ethyl acetate/hexane) to give the desired product 5-bromo-1-methyl-1H-tetrazole as a white solid (30 mg, 31.1% yield). ¹H NMR (400 MHZ, DMSO-d₆) δ: 4.05 (s, 3H); LC-MS (M+1)⁺: 163.

Step C. Synthesis of 5-hydrazinyl-1-methyl-1H-tetrazole.

To a stirred solution of 5-bromo-1-methyl-1H-tetrazole (30 mg, 0.186 mmol) in iPrOH(1 mL) was added Hydrazine hydrate (1 ml). The reaction mixture was stirred at 80° C. overnight. The reaction mixture was partitioned between EtOAc (10 mL) and water (10 mL). The liquid phase was concentrated to give the desired compound 5-hydrazinyl-1-methyl-1H-tetrazole (7.3 mg, 34.4% yield) as yellow solid. ¹H NMR (400 MHZ, DMSO-d₆) δ: 8.96 (brs, ¹H), 4.12 (d, J=4.0 Hz, 1H), 2.24 (s, 3H); LC-MS (M+1)⁺: 115.

### Example 26

### Synthesis of 2-(1-(1H-tetrazol-5-yl)hydrazinyl)ethan-1-ol

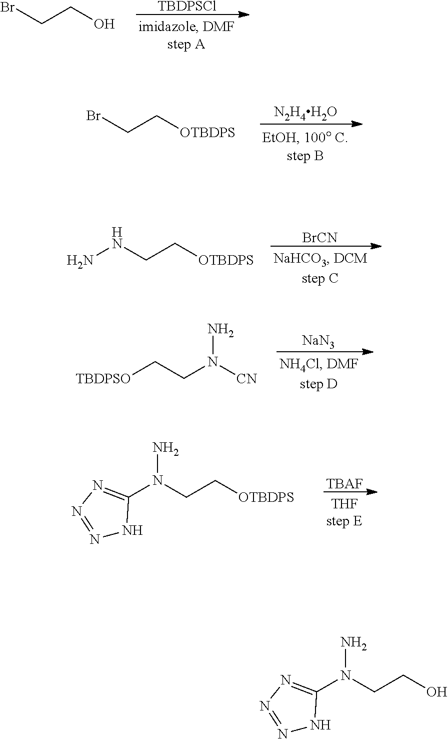

Step A. Synthesis of (2-bromoethoxy)(tert-butyl)diphenylsilane.

A solution of 2-bromoethan-1-ol (1.0 g, 8.0 mmol), imidazole (1.1 g, 16.0 mmol) and TBDPSCI (2.42 g, 8.8 mmol) in DMF (20 mL) was stirred at room temperature for 8 h. The reaction mixture was diluted with water (80 mL) and extracted with ethyl acetate (2×80 mL). The combined organic layers were washed with brine (2×80 mL), dried over Na₂SO₄, concentrated under reduced pressure and purified by column chromatography (Silica gel 100-200, 100:1, ethyl acetate/hexane) to give the desired product (2-bromoethoxy)(tert-butyl)diphenylsilane as a colorless oil (3.0 g, 95% yield). LCMS (M+1)⁺: 363.

Step B. Synthesis of (2-((tert-butyldiphenylsilyl)oxy)ethyl)hydrazine.

A solution of (2-bromoethoxy)(tert-butyl)diphenylsilane (3.0 g, 8.3 mmol) and N₂H_{4.}H₂O (5.3 g, 82.8 mmol, wt:80% in H₂O) in EtOH (10 mL) was stirred at 100° C. for 16 h. The reaction mixture was diluted with aqueous NaHCO₃ (80 mL) and extracted with DCM (2×80 mL). The combined organic layers were washed with brine (2×80 mL), dried over Na₂SO₄, concentrated under reduced pressure and purified by column chromatography (Silica gel 100-200, 1:1, ethyl acetate/hexane) to give the desired product (2-((tert-butyldiphenylsilyl)oxy)ethyl)hydrazine as a yellow oil (1.4 g, 42% yield). LCMS (M+1)⁺: 315.

Step C. Synthesis of (2-((tert-butyldiphenylsilyl)oxy)ethyl)hydrazine.

To a mixture of (2-((tert-butyldiphenylsilyl)oxy)ethyl)hydrazine (1.4 g, 4.5 mmol) and aqueous NaHCO₃ (750 mg, 8.9 mmol, 2mmol/mL) in DCM (10 mL) was added BrCN (470 mg, 4.5 mmol) in DCM at 0° C. Then the mixture was stirred at room temperature for 30 mins. The reaction mixture was diluted with water (50 mL) and extracted with DCM (2×50 mL). The combined organic layers were washed with brine (2×50 mL), dried over Na₂SO₄, concentrated under reduced pressure and purified by column chromatography (Silica gel 100-200, 1:2, ethyl acetate/hexane) to give the desired product 1-(2-((tert-butyldiphenylsilyl)oxy)ethyl)hydrazine-1-carbonitrile as a colorless oil (900 mg, 60% yield). LCMS (M+1)⁺: 340.

Step D. Synthesis of 5-(1-(2-((tert-butyldiphenylsilyl)oxy)ethyl)hydrazinyl)-1H-tetrazole.

A mixture of 1-(2-((tert-butyldiphenylsilyl)oxy)ethyl)hydrazine-1-carbonitrile (950 mg, 2.8 mmol), NH₄Cl (450 mg, 8.4 mmol) and NaN₃ (365 mg, 5.6 mmol) in DMF (10 mL) was stirred at 90° C. for 16 h. After completion of the reaction, aqueous critic acid solution was added and extracted with ethyl acetate (2×30 mL). The combined organic layers were washed with brine (1×30 mL), dried over Na₂SO₄, concentrated under reduced pressure and purified by prep-HPLC to give the desired compound 5-(1-(2-((tert-butyldiphenylsilyl)oxy)ethyl)hydrazinyl)-1H-tetrazole as a white solid (350 mg, 35%yield). LCMS (M+1)⁺: 383.

### Step D. Synthesis of 2-(1-(1H-tetrazol-5-yl)hydrazinyl)ethan-1-ol.

To a solution of 5-(1-(2-((tert-butyldiphenylsilyl)oxy)ethyl)hydrazinyl)-1H-tetrazole (350 mg, 0.92 mmol) in THF was added TBAF (1 mL, 1.0 mmol, 1.0 M in THF). Then the mixture was stirred at room temperature for 2 h. Then the mixture was concentrated under reduced pressure and purified by prep-HPLC to give the desired product 2-(1-(1H-tetrazol-5-yl)hydrazinyl)ethan-1-ol as a white solid (50 mg, 35% yield). ¹H NMR (400 MHZ, DMSO-d₆) δ: 8.37 (s, 1H), 3.66 (t, J=6.0 Hz, 1H), 3.49 (t, J=6.0 Hz, 1H); LCMS (M+1)⁺: 145.

### Example 27

### Synthesis of tert-butyl ethyl 2-hydrazinyl-4-methylthiazole-5-carboxylate

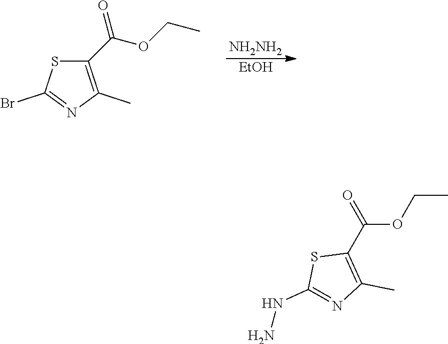

To a solution of ethyl 2-bromo-4-methylthiazole-5-carboxylate (500 mg ,2 mmol) in EtOH (20 mL) was added Hydrazine hydrate (0.5 mL). The resulting mixture and stirred at 70° C. for 16 hrs. The reaction mixture was evaporated to dryness and the residue was purified by flash column chromatography on silica gel (PE/EA=3:1) to afford tert-butyl ethyl 2-hydrazinyl-4-methylthiazole-5-carboxylate (200 mg, 50% yield) as a white solid. ¹H NMR (400 MHZ, DMSO-d₆) δ 9.27 (s, 1H), 5.05 (s, 2H), 4.14 (d, J=7.1 Hz, 2H), 2.37 (s, 3H), 1.23 (t, J=7.1 Hz, 3H); LCMS (M+1)⁺: 202.

### Example 28

### Synthesis of 5-(1-propylhydrazinyl)-1H-tetrazole

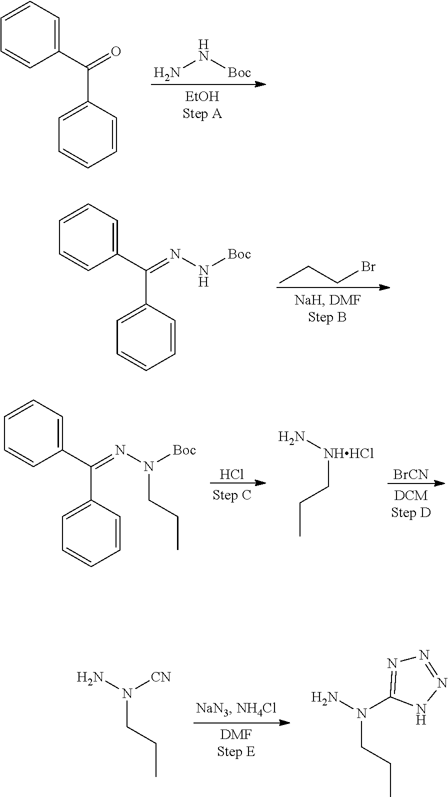

Step A. Synthesis of tert-butyl 2-(diphenylmethylene)hydrazinecarboxylate

The mixture of benzophenon (2.0 g, 10.9 mmol), tert-butyl hydrazinecarboxylate (1.45 g, 10.9 mmol) and AcOH (1 ml) in EtOH (10 ml) was heated at 50° C.for 2 h. The mixture was concentrated to remove solvent, then the residue was purified by column chromatography to give tert-butyl 2-(diphenylmethylene)hydrazinecarboxylate as a white solid (3.0 g, 93.0% yield). LCMS (M+1)⁺: 297.

Step B. Synthesis of tert-butyl 2-(diphenylmethylene)-1-propylhydrazine carboxylate

To a solution of tert-butyl 2-(diphenylmethylene)hydrazinecarboxylate (500 mg, 1.68 mmol) in DMF (3 mL) was added NaH (101 mg, 2.53 mmol) at 0° C.for 5 min, then 1-bromopropane (311 mg , 2.53 mmol) was added to the solution. The solution was stirred at 50° C. for 2 h. The solution was poured into ice water and extracted with EtOAc twice. The combined organic phase was washed with brine, dried over NaSO₄, concentrated and purified by column chromatography to give tert-butyl 2-(diphenylmethylene)-1-propylhydrazinecarboxylate (550 mg, 96.8% yield). LCMS (M+1)⁺: 339.

Step C. Synthesis of propylhydrazine hydrochloride

A solution of tert-butyl 2-(diphenylmethylene)-1-propylhydrazinecarboxylate (3 g, 8.87 mmol) in MeOH (30 mL) was added con. HCl (10 ml) was stirred at r.t for 1 h. The mixture was concentrated to remove solvent and EtOAc was added to the mixture. The mixture was stirred for 10 min, then filtrated to give propylhydrazine hydrochloride as a white solid (810 mg, 83.0% yield). LCMS (M+1)⁺: 75.

Step D. Synthesis of 1-propylhydrazinecarbonitrile.

To a solution of propylhydrazine hydrochloride (500 mg, 4.54 mmol) in DCM was added a solution of Na₂CO₃ (1.25 g, 9.08 mmol) and BrCN (481 mg, 4.54 mmol) in H₂O (5 ml) solwly at 0° C., then the mixture was stirred at 0° C.for 1 h. The mixture was added H₂O and extracted with DCM twice, then the combined organic phase was concentrated to afford 1-propylhydrazinecarbonitrile (300 mg, 67% yield) as a yellow oil. LCMS (M+1)⁺: 100.

Step D. Synthesis of 5-(1-propylhydrazinyl)-1H-tetrazole

To a solution of 1-propylhydrazinecarbonitrile (300 mg, 3.03 mmol) in DMF (3 mL) was added NaN₃ (196 mg, 3.03 mmol) and NH₄Cl (160 mg, 3.03 mmol). Then the mixture was heated at 90° C. for 2 h. The mixture was filtrated and the filtrate was concentrated to removed solvent, then purified by Al2O₃ column chromatography (DCM/MeOH/NH₃.H₂O=4/1/0.2) to give 5-(1-propylhydrazinyl)-1H-tetrazole (150 mg, 34.8% yield) as a white solid. ¹H NMR (400 MHZ, DMSO-d₆) δ 3.41 (t, J=7.2 Hz, 2H), 2.50 (dt, J=7.2Hz, 7.6 Hz, 1H), 0.87 (t, J=7.2 Hz, 3H); LCMS (M+1)⁺: 143.

### Example 29

### Synthesis of 5-(1-cvclopropvlhydrazinvl)-1H-tetrazole

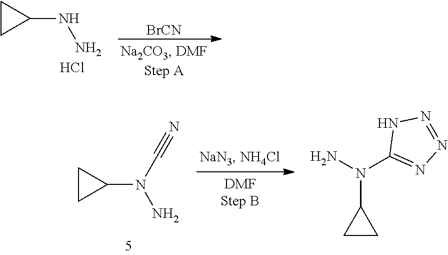

Step A. Synthesis of 1-cyclopropylhydrazine-1-carbonitrile.

To a solution of cyclopropylhydrazine hydrochloride (300 mg, 2.76 mmol) in DMF (3 mL) was added a Na₂CO₃ (439 mg, 4.14 mmol) and BrCN (439 mg, 4.14 mmol) solwly at 0° C., then the mixture was stirred at rt for 1 h. The mixture was added H₂O and extracted with DCM twice, then the combined organic phase was concentrated to afford 1-cyclopropylhydrazine-1-carbonitrile (150 mg, 56% yield) as a yellow oil. LCMS (M+1)⁺: 98.

Step B. Synthesis of 5-(1-cyclopropylhydrazinyl)-1H-tetrazole

To a solution of 1-cyclopropylhydrazine-1-carbonitrile (150 mg, 1.55 mmol) in DMF (3 mL) was added NaN₃ (202 mg, 3.10 mmol) and NH₄Cl (166 mg, 3.10 mmol). Then the mixture was heated at 90° C. for 2 h. The mixture was filtrated and the filtrate was concentrated to removed solvent, then purified by Prep-HPLC to give 5-(1-cyclopropylhydrazinyl)-1H-tetrazole (10 mg, 5% yield) as a white solid. ¹H NMR (400 MHZ, DMSO-d₆) δ 4.99 (s, 2H), 2.83-2.80 (m, 1H), 0.80-0.71 (m, 4H); LCMS (M+1)⁺: 141.1.

### Example 30

### Synthesis of 5-(1-isopropylhydrazinyl)-1H-tetrazole

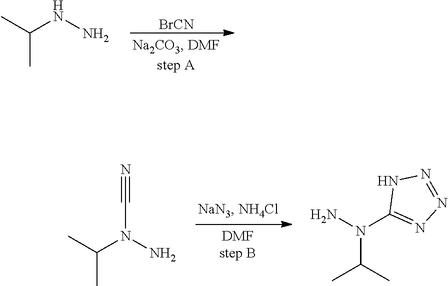

Step A. Synthesis of 1-isopropylhydrazine-1-carbonitrile

To a solution of isopropylhydrazine (3.0 g, 40.47 mmol) in DMF (4 mL) was added a Na₂CO₃ (6.4 g, 60.70 mmol) and BrCN (6.4 g, 60.70 mmol) solwly at 0° C., then the mixture was stirred at rt for 3 h. The mixture was added H₂O and extracted with DCM twice, then the combined organic phase was concentrated to afford 1-isopropylhydrazine-1-carbonitrile (1.4 g, 35% yield) as a yellow oil. LCMS (M+1)⁺: 101.1.

Step B. Synthesis of 5-(1-isopropylhydrazinyl)-1H-tetrazole

To a solution of 1-isopropylhydrazine-1-carbonitrile (300 mg, 3.03 mmol) in DMF (3 mL) was added NaN₃ (394 mg, 6.06 mmol) and NH₄Cl (324 mg, 6.06 mmol). Then the mixture was heated at 90° C. for 2 h. The mixture was filtrated and the filtrate was concentrated to removed solvent, then purified by Prep-HPLC to afford 5-(1-isopropylhydrazinyl)-1H-tetrazole (50 mg, 12% yield) as a white solid. ¹H NMR (400 MHZ, DMSO-d₆) δ 14.53 (s, 1H), 4.60 (s, 2H), 4.22-4.15 (m, 1H), 1.11 (d, J=6.4 Hz, 6H); LCMS (M+1)⁺: 143.1.

### Example 31

### Synthesis of 5-(1-ethylhydrazinyl)-1H-tetrazole

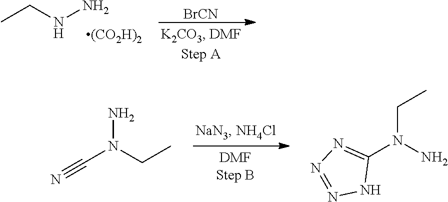

Step A. Synthesis of 1-ethylhydrazine-1-carbonitrile.

To a solution of cyanogen bromide (500 mg, 4.72 mmol) in DMF (10 mL) was added ethylhydrazine Oxalate (709 mg, 4.72 mmol) and sodium carbonate (1.50 g, 14.16 mmol) at 0° C. The reaction mixture was stirred at this temperature for 3 h. Then diluted with EtOAc (60 mL). The organic phase was washed with water (15 mL) and brine (15 mL), dried over Na₂SO₄, filtered, concentrated to dryness and the residue was purified by flash column chromatography on silica gel (EtOAc/PE=1:1) to afford 1-ethylhydrazine-1-carbonitrile (300 mg, 75% yield) as a colorless oil. LCMS (M+1)⁺: 86.

Step B. Synthesis of 5-(1-ethylhydrazinyl)-1H-tetrazole.

A mixture of 1-ethylhydrazine-1-carbonitrile (300 mg, 3.52 mmol), sodium azide (458 mg, 7.05 mmol) and ammonium chloride (566 mg, 10.57 mmol) in DMF (6 mL) was stirred at 90° C. for 1 h. The resulting mixture was filtered and evaporated. The residue was purified by Prep-TLC (DCM:MeOH:NH₃OH=4:1:0.2) to afford 5-(1-ethylhydrazinyl)-1H-tetrazole (25 mg, 5.54%yield) as a white solid. ¹H NMR (400 MHZ, DMSO-d₆) δ 3.48 (q, J=8.0 Hz, 2H), 1.14 (t, J=8.0 Hz, 3H). LCMS (M+1)⁺: 129.

### Example 32

### Synthesis of 3-(1-(1H-tetrazol-5-yl)hydrazinyl)propan-1-ol

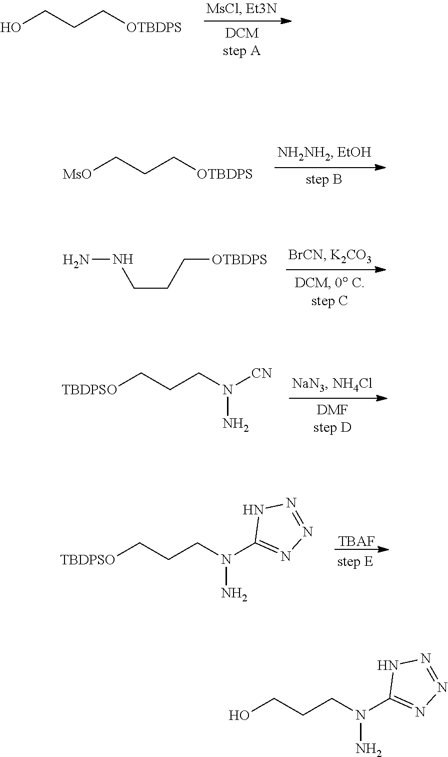

Step A. Synthesis of 3-((tert-butyldiphenylsilyl)oxy)propyl methanesulfonate

To a solution of 3-((tert-butyldiphenylsilyl)oxy)propan-1-ol (7.0 g, 22.26 mmol) in DCM (50 mL) was added Et3N (3.37 g, 33.39 mmol) and MsCl (2.8 g, 24.48 mmol) at 0° C. The reaction mixture was stirred at rt for 3 h. The mixture was washed with H₂O and extracted with EtOAc. Evaporated and purified by flash chromatography (Silica gel 60-120, ethyl acetate/hexane=1:4) to afford 3-((tert-butyldiphenylsilyl)oxy)propyl methanesulfonate as a yellow oil (8.0 g, 92% yield). LCMS (M+1) : 393.

Step B. Synthesis of (3-((tert-butyldiphenylsilyl)oxy)propyl)hydrazine

To a solution of 3-((tert-butyldiphenylsilyl)oxy)propyl methanesulfonate (8.0 g, 25 mmol) in ethanol (15 mL) was added hydrazine monohydrate (15 mL) and the mixture was heated to 60° C.for 2 h then concentrated to afford (3-((tert-butyldiphenylsilyl)oxy)propyl)hydrazine (5.0 g, 75% yield). LCMS (M+1)⁺: 329.

Step C. Synthesis of 1-(3-((tert-butyldiphenylsilyl)oxy)propyl)hydrazine-1-carbonitrile

The solution of BrCN (2.42 g, 22.83 mmol) in DCM (20 ml) was cooled to 0° C., A mixture of (3-((tert-butyldiphenylsilyl)oxy)propyl)hydrazine (5.0 g, 15.22 mmol), Na₂CO₃ (2.42 g, 22.83 mmol) and 10 ml H₂O was added dropwise under vigorous stirring for 1 h. The solution was extracted with DCM, concentrated and purified by column chromatography to give 1-(3-((tert-butyldiphenylsilyl)oxy)propyl)hydrazinecarbonitrile as a yellow oil (3.5 g, 65% yield). LCMS (M+1)⁺: 354.

Step D. Synthesis of 5-(1-(3-((tert-butyldiphenylsilyl)oxy)propyl)hydrazinyl)-1H-tetrazole

To a solution of 1-(3-((tert-butyldiphenylsilyl)oxy)propyl)hydrazinecarbonitrile (0.5 g, 1.41 mmol) in DMF (5 mL) was added NaN₃ (92 mg, 1.41 mmol) and NH₄Cl(75 mg, 1.41 mmol). Then the reaction mixture was heated at 90° C.for 1.5 h. The mixture was filtrated, the filtrate was concentrated. The residue was purified by column chromatography to give 5-(1-(3-((tert-butyldiphenylsilyl)oxy)propyl)hydrazinyl)-1H-tetrazole as a white solid (180 mg, 32% yield). LC-MS (M+1)⁺: 397.

### Step E. Synthesis of 3-(1-(1H-tetrazol-5-yl)hydrazinyl)propan-1-ol

To a solution of 5-(1-(3-((tert-butyldiphenylsilyl)oxy)propyl)hydrazinyl)-1H-tetrazole (100 mg, 0.25 mmol) in THF (3 mL) was added TBAF (0.5 mL, 1 M) and then stirred overnight at rt. The mixture was evaporated and purified by Prep-HPLC to afford 3-(1-(1H-tetrazol-5-yl)hydrazinyl)propan-1-ol (10 mg, 25% yield). ¹H NMR (400 MHZ, D₂O) 8: 3.61 (t, J=6.4 Hz, 2 H), 3.22 (t, J=7.2 Hz, 2 H), 1.77-1.73 (m, 2 H); LC-MS (M+1)⁺: 159.

### Example 33

### Synthesis of 4-(1-(1H-tetrazol-5-yl)hydrazinyl)butan-1-ol

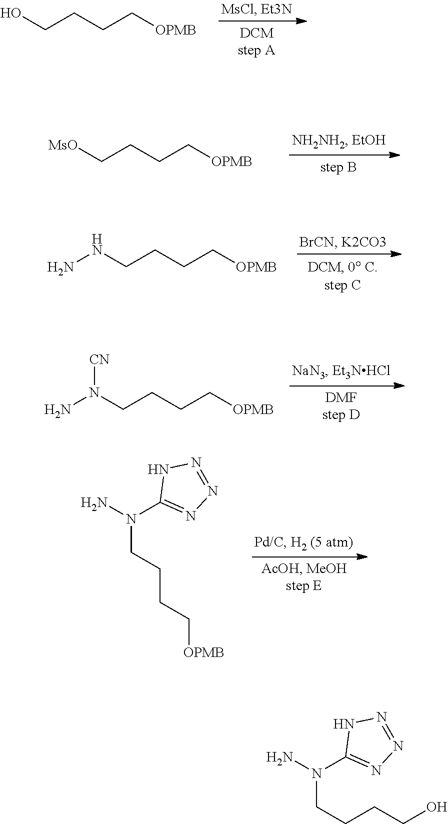

Step A. Synthesis of 4-((4-methoxybenzyl)oxy)butyl methanesulfonate

To a solution of 4-((4-methoxybenzyl)oxy)butan-1-ol (4.0 g, 19.0 mmol) in DCM (50 mL) was added Et3N (2.88 g, 28.5 mmol) and MsCl (2.4 g, 20.9 mmol) at 0° C. The reaction mixture was stirred at rt for 3 h. The mixture was washed with H₂O and extracted with EtOAc. Evaporated and purified by flash chromatography (Silica gel 60-120, ethyl acetate/hexane=1:4) to afford 4-((4-methoxybenzyl)oxy)butyl methanesulfonate as a yellow oil (4.0 g, 73% yield). LC-MS (M+1)⁺: 289.

Step B. Synthesis of (4-((4-methoxybenzyl)oxy)butyl)hydrazine

To a solution of afford 4-((4-methoxybenzyl)oxy)butyl methanesulfonate (4.0 g, 13.9 mmol) in ethanol (15 mL) was added hydrazine monohydrate (15 mL) and the mixture was heated to 60° C.for 2 h then concentrated to afford (4-((4-methoxybenzyl)oxy)butyl)hydrazine (1.6 g, 52% yield) as a yellow oil. LCMS (M+1)⁺: 225.

Step C. Synthesis of 1-(4-((4-methoxybenzyl)oxy)butyl)hydrazinecarbonitrile

The solution of BrCN (1.13 g, 10.7 mmol) in DCM (20 ml) was cooled to 0° C., A mixture of (4-((4-methoxybenzyl)oxy)butyl)hydrazine (1.6 g, 7.1 mmol), Na₂CO₃ (1.13 g, 10.7 mmol) and 10 ml H₂O was added dropwise under vigorous stirring for 1 h. The solution was extracted with DCM, concentrated and purified by column chromatography to give 1-(4-((4-methoxybenzyl)oxy)butyl)hydrazinecarbonitrile as a yellow oil (1.0 g, 56% yield). LC-MS (M+1)⁺: 250.

Step D. Synthesis of 5-(1-(4-((4-methoxybenzyl)oxy)butyl)hydrazinyl)-1H-tetrazole

To a solution of 1-(4-((4-methoxybenzyl)oxy)butyl)hydrazinecarbonitrile (1.0 g, 4.01 mmol) in DMF (10 mL) was added NaN₃ (391 mg, 6.02 mmol) and NH₄Cl (322 mg, 6.02 mmol). Then the reaction mixture was heated at 90° C.for 1.5 h. The mixture was filtrated, the filtrate was concentrated. The residue was purified by column chromatography to give 5-(1-(4-((4-methoxybenzyl)oxy)butyl)hydrazinyl)-1H-tetrazole as a white solid(250 mg, 21% yield). LC-MS (M+1)⁺: 293.1.

### Step E. Synthesis of 4-(1-(1H-tetrazol-5-yl)hydrazinyl)butan-1-ol

To a solution of 5-(1-(4-((4-methoxybenzyl)oxy)butyl)hydrazinyl)-1H-tetrazole (60 mg, 0.21 mmol) in MeOH (3 mL) and AcOH (1 mL) was added Pd/C (10 mg). The resulting mixture was stirred overnight at rt under H₂ (6 atm) atmosphere. The mixture was filtered, the filtrate was concentrated and purified by Prep-HPLC to afford 4-(1-(1H-tetrazol-5-yl)hydrazinyl)butan-1-ol (5 mg, 14% yield). ¹H NMR (400 MHZ, D2O) δ: 3.56-3.49 (m, 4 H), 1.66-1.63 (m, 2 H), 1.51-1.47 (m, 2 H); LC-MS (M+1)⁺: 173.

### Example 34

### Synthesis of 5-(1-methylhydrazinyl)-1H-tetrazole

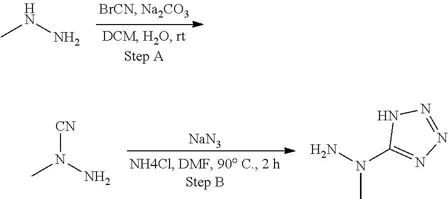

Step A. Synthesis of 1-(methyl-d3)hydrazine-1-carbonitrile

The solution of BrCN (13.8 g, 130 mmol) in DCM (300 ml) was cooled to 0ºC, A mixture of methyl hydrazine (6 g, 40% in H₂O), Na₂CO₃ (2.07 g, 195 mmol) and 60 ml H₂O was added dropwise under vigorous stirring. The solution was extracted with DCM, concentrated and purified by column chromatography to give 1-(methyl-d3)hydrazine-1-carbonitrile as a yellow oil (2.7 g, 29.2% yield). LCMS (M+1)⁺: 72.

Step B. Synthesis of 5-(1-methylhydrazinyl)-1H-tetrazole

To a solution of 1-(methyl-d3)hydrazine-1-carbonitrile (4.55 g, 64.1 mmol) in DMF (15 mL) was added NaN₃(4.16 g, 64.1 mmol) and NH₄Cl(3.43 g, 64.1 mmol). Then the reaction mixture was heated at 90° C.for 1.5 h. The mixture was filtrated, the filtrate was concentrated. The residue was recrystallize from EtOH/H₂O=1/1 to give desired product as a white solid (3.5 g, 47.8% yield). ¹H NMR (400 MHZ, DMSO-d₆) δ 14.62 (s, 1H), 4.98 (s, 2H), 3.14 (s, 5H); LCMS (M+1)⁺: 115.

### Example 35

### Synthesis of 1-benzyl-5-(1-methylhydrazinyl)-1 H-tetrazole

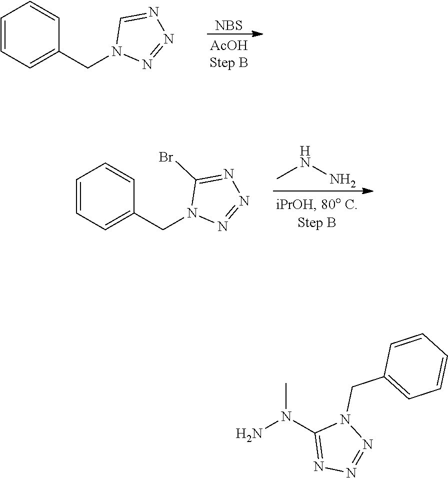

Step A. Synthesis of 1-benzyl-5-bromo-1H-tetrazole.

To a stirred solution of 1-benzyl-1H-tetrazole (600 mg, 3.7 mmol) in AcOH (5 mL) was added NBS (796 mg, 4.5 mol) at room temperature. The reaction mixture was stirred at 85° C.for 6 h and concentrated under reduced pressure. The residue was purified by column chromatography (Silica gel 60-120, 1:3, ethyl acetate/hexane) to give the desired product 1-benzyl-5-bromo-1H-tetrazole as a white solid (560 mg, 63.0% yield). LC-MS (M+1)⁺: 239.

Step B. Synthesis of 1-benzyl-5-(1-methylhydrazinyl)-1H-tetrazole.

To a stirred solution of 1-benzyl-5-bromo-1H-tetrazole (560 mg, 2.4 mmol) in iPrOH (5 mL) was added Methylhydrazine (5 ml, wt: 30% in water) at room temperature. The reaction mixture was stirred at 80° C.overnight. After completion of the reaction, the reaction mixture was diluted with ice water and extracted with DCM (2×20 mL). The combined organic layers were washed with brine (1×20 mL), dried over Na₂SO₄ and concentrated under reduced pressure to give the desired compound, 1-benzyl-5-(1-methylhydrazinyl)-1H-tetrazole as a white solid (340 mg, 70.9% yield). ¹H NMR (400 MHZ, DMSO-d₆) δ: 7.35-7.23 (m, 5H), 5.80 (s, 2H), 4.91 (s, 2H), 3.15 (s, 3H); LCMS (M+1) : 205.

### Example 36

### Validation of ALAS2 as a Target for Inhibition for Treatment Of Porphyrias

The objective of this series of experiments was to explore and validate ALAs2 inhibition as an effective intervention strategy to treat erythroporphyrias (XLP, EPP and CEP). This was achieved by experimental verification of ALAS2 being the rate-limiting enzyme in the heme biosynthetic pathway. Thus, inhibition of ALAS2 or reduction in ALAS2 protein level is expected to reduce pathway flux that should suppress the production of toxin metabolites such as PPIX, uroporphyrin I or coproporphyrin I, or heme and help ameliorate all three erythroporphyrias as well as Del5q MDS and DBA, two anemia diseases that are thought to be caused by accumulation of excess heme. Additionally, in vivo target validation was achieved by genetic knockdown of ALAS2 with delAT mutation in a novel XLP mouse model. Thus, ALAS2 could be inhibited by a small molecule in the XLP mouse model to decrease bone marrow ALA and PPIX.

### 36.1. Mass Isotopomer Distribution of PPIX Using ¹³C-glycine

TF-1 cells, maintained in 10% FBS RPMI medium with 2ng/ml GM-CSF (R&D Systems), were transduced with a lentivirus to introduce a shRNA against human FECH with a puromycin marker (shRNA sequence: GACCATATTGAAACGCTGTAT). Erythroid differentiation of these TF-1 cells with FECH knocked down was induced by maintaining the cells in 10% FBS RPMI medium with 5U/ml erythropoietin (R&D Systems). After 6 days of in vitro differentiation, cells were placed in specially formulated RPMI medium without glycine, supplemented with 10% dialized FBS, 0.2 mM ¹³C₂-glycine, and 5U/ml erythropoietin. At different timepoints, 0.5×10⁶ cells were spun down at 14,000 rpm at 4° C. and lyzed immediately with 80% methanol/20% water on dry ice. Insoluble matters were removed by centrifugation. The liquid layer was dried down under vacuum prior to LC-MS analysis. Porphyrins were separated using a Thermo Hypersil Gold Column (50×2.1 mm, 1.9 um particle size). Mobile phase A consisted of H₂₀ with 0.1% formic acid and mobile B consisted of acetonitrile with 0.1% formic acid. The gradient at a flow rate of 400 AL min-1 was applied as follows: 0-1 min 30% B, 1-6 min 98% B, 6-7 min 98% B, 7-7.1 min 30%B, 7.1-8.5 min 30% B. LC/MS was conducted using a Thermo Vanquish Flex pump coupled to a Thermo QExactive Mass Spectrometer operated in positive ESI, full scan mode. Amino-levulinic acid was separated using a Thermo Vanquish Flex pump delivered a gradient of 0.025% heptafluorobutyric acid, 0.1% formic acid in water and acetonitrile at 400 μl min−1 (PMID: 20349993). The stationary phase was an Atlantis T3, 3 μm, 2.1 mm×150 mm column. Data was acquired on a QExactive mass spectrometer operated at 70,000 resolving power in full-scan ESI positive mode (PMID: 20349993).

36.2. Generation of XLP Mouse Model with ALAS2-delAT Mutation

Materials for microinjection: For in vitro transcription of sgRNA, a DNA template was amplified from a plasmid containing the sgRNA sequence by PCR, using T7 promoter tagged primers (T7-sgRNA_F: ttaatacgactcactataggCTTTGAACTTATGAGCGAGT, sgRNA_R: AAAAGCACCGACTCGGTGCC (SEQ ID NO:4)). After purification, in vitro transcription was performed using the MEGAshortscript T7 kit and MEGAclear kit according to the protocol (Ambion). Cas9 mRNA was in vitro transcribed from pUC-cas9 vector using mMESSAGE mMACHINE T7 kit (Ambion). To introduce the mutation, a ssDNA oligo with the desired alteration flanked on each side by ˜80 bases homologous to the sequence adjoining the double-stranded break was synthesized. To prevent DNA being cut after HDR and provide a SacI restriction site for genotyping, several additional silent mutations were also introduced: ssDNA sequence

(SEQ ID NO: 5)
CTGGCCTGGACTGAGGTGGGGCTGCCCCTCCAAGATGTGTCTGTG
GCTGCATGCAACTTCTGTCATCGTCCTGTGCACTTTGAgCTcGAa
tGAGTGGGAGCGATCCTACTTTGGGAACATGGGACCCCAATATGT
TACCACCTATGCTTAAGGAGCCAGCTGCCTTGGATGCCAGCTCCA

Pronuclear injection: 10˜15 C₅₇BL/6 females (4-week-old) were injected with PMSG (5IU-10IU) on day 1. 48 hours later, the mice were injected with HCG (5IU-10IU) and then housed with C₅₇BL/6 male overnight. In the morning of day 4, female mice with copulation plugs were collected for zygote preparation. Zygote-cumulus complexes from the oviduct were collected from euthanized mice and suspended in hyaluronidase solution for several minutes until the cumulus cells fell off, which were then washed several times in M2 medium. The embryos were then placed in equilibrated M16 medium (medium covered with mineral oil) at 37° C.in a 5% CO₂ incubator. Cas9 mRNA, sgRNA, and ssDNA (all 10ng/ul) were injected into the pronucleus of the zygotes. Injected zygotes were then cultured in M16 or KSOM medium at 37° C.in a 5% CO₂ incubator until two-cell stage, at which point they were implanted into the oviduct of pseudo-pregnant foster mothers at 0.5 dpc. As ALAS2 is an X-linked gene, the following symbols are used to describe mice carrying the ALAS2-delAT mutation: ALAS2^{delAT/Y} (male); ALAS2^{delAT/WT} or ALAS2^{delAT/delAT} (female).

Genotyping: Genomic DNAs were extracted from tails and toes of the 7-day-old pups and used in PCR amplification of the sequence around the targeting site. The resultant PCR products were TA cloned and sequenced to identify Fo mice, which were back crossed with wildtype C₅₇BL/6 mice to identify F1 mice with germline transmitted mutation.

### 36.3. Generation of a Mouse Line in Which ALAS2 Protein Can be Knocked Down in an Inducible Manner Via Doxycycline Treatment.

This mouse line was custom ordered from Mirimus, Inc (NY; https://www.mirimus.com/). The following shRNA sequence was used to target both wildtype mouse ALAS2 and mutant ALAS2 with delAT mutation: UGAAAAAUUGGUCAUAACCGAA (SEQ ID NO:6). This mouse line carries the expression cassette of the reverse tetracycline-controlled transactivator (rtTA) under a Rosa26 promoter on chromosome 6. In addition, the expression cassette for the ALAS2-shRNA (shALAS2) under the TRE promoter was knocked into chromosome 11 at the ColAl locus. Expression of ALAS2 shRNA could be induced by treatment of the mice with doxycycline in food to knock down ALAS2 or ALAS2-delAT protein expression. The following symbols are used to described mice carrying these two transgenes: rtTA^{+/-} (heterozygous); rtTA^{+/+} (homozygous); shALAS2^{+/-} (heterozygous); shALAS2^{+/+} (homozygous).

Mice were first bred to homozygosity (rtTA^{+/-}/shALAS2^{+/+}). To demonstrate shRNA-mediated ALAS2 protein knockdown, 8 mice were randomized into two groups based on weight (n=4 each). One group was fed with regular chow and the other with chow containing 500 mg/kg doxycycline for 4 days. Mice were then euthanized. To collect bone marrow tissues, both femurs and tibias were harvested. Muscles and all connective tissues were removed. With both ends of the bones snipped off, each femur/tibia set (2 sets per mouse) was placed in a 2 ml Eppendorf tube stacked end-to-end and spun at 4° C. for 10 mins at ×13,000 g. Bones were then carefully removed. The bone marrow tissue pellets was used for western blot to measure ALAS2-delAT protein level. Briefly, the pellets were dissolved in RIPA buffer. Equal volume of lysates were separated by electrophoresis on a NuPAGE 4-12% Bis-Tris gel (Invitrogen). Proteins were visualized by western blotting using the following antibodies. The ALAS2 antibody is a mouse monoclonal antibody against an internal region of ALAS2 protein (DPDHLKKLLEKSNPKI (SEQ ID NO:7)). This ALAS2 antibody was validated to only recognize ALAS2 but not ALAS1. The actin antibody is a rabbit antibody from Cell Signaling.

To demonstrate shRNA gene dosage effect on ALAS2 protein knockdown, the ALAS2 protein level in mice was compared with one or two copies of shALAS2 after doxycycline treatment. In one experiment, 3 WT C₅₇B6 mice and 4 rtTA^{+/-}/shALAS2^{+/-} mice were fed with doxycycline-containing chow for 16 days. Another experiment was set with 3 WT C₅₇B6 mice and 4 rtTA^{+/+}/shALAS2^{+/+} mice also being fed with doxycycline-containing chow for 16 days. Animals were euthanized at the end of the experiment. Bone marrow ALAS2 protein was analyzed as above.

### 36.4. In Vivo Experiments.

The mouse line generated from Examples 36.2 and 36.3 were crossed to generate a mouse line with the following genotype: ALAS2^{delAT/Y}; rtTA^{+/-}; shALAS2^{+/-}. This XLP line expressed the disease causing ALAS2-delAT mutation but also harbored one copy of the rtTA and shALAS2 transgene. Upon doxycycline treatment, enough of shALAS2 was expressed to knock down ˜50% of ALAS2-delAT protein.

Briefly, 17 ALAS2^{delAT/Y}; rtTA^{+/-}; shALAS2^{+/-} were randomized into two groups by weight and blood PPIX fluorescence. Eight were kept on normal chow and nine on chow with 500 mg/kg doxycycline. A control group of age-matched WT C₅₇B6 mice on dox diet was included to provide a baseline ALA and PPIX values in non-disease mice. The study was carried out for 39 day. Mice were then euthanized. Whole blood was collected and analyzed for PPIX as described in Example 36.6. To collect bone marrow tissues, both femurs and tibias were harvested. Muscles and all connective tissues were removed. With both ends of the bones snipped off, each femur/tibia set (2 sets per mouse) was placed in a 2 ml Eppendorf tube stacked end-to-end and spun at 4 oC for 10 mins at ×13,000 g. Bones were then carefully removed. One set of the bone marrow tissue pellets was used for measuring ALA. The other set of the bone marrow tissue pellets was used for western blot to measure ALAS2-delAT protein level as described in Example 36.3.

### 36.5 Hydrazine Compounds Inhibits PPIX Production in EPP and XLP In Vitro Cell Models.

### In Vitro EPP Cell Model Using Engineered A549 Cells

Endogenous ALAS1 in A549 cells was knocked out by CRISPR/CAS9 using the Targeting System from SBI System Biosciences. The following two homologous arms were cloned into pHR⁵¹⁰PA-1 vector to target exon 4 of human ALAS1:

homologous arm 1:
(SEQ ID NO: 8)
GTGAGGCTGGGCACAGTAGCTCATACCTGTAATCCCAGCACTTTG
GGAGGCTGAAGCGGGAGAAGATCACTTGAGGCCAGGAGTTTGAGG
CCAGCCTTGGCAACATAGCAAGACACTATCTCTACCAAAACAGTT
TTTTTAATTAGCCAGGCATGATGGCTTGCATCTATAGTCCCAGCT
ACTCAGGAGGCTAAGGTAGGAAGATTGCTTAAGCCCAAGACTTCA
AGGTTCAGTGAGCTATGATCACGCTATTGCACTCCAGCCTGAATG
ACAGACAGAGACTCAGTTTCTAGGAAAAACAAAAAAATGTATGGT
GAGTTGAGGCTTGAAAGCCATATCCCTTGCTTGCGTGAGCAGGTG
CTTTTGGTTGTGGTGACTACAGGTGCTGTTGGTAGCCTGCCTTCT
GTCCTTTAATACTTACCGTCTACACATGGCACTGAGCCAAACACG
ACATACAGTCTTATTTACTTACAAGATGAGGACATCAAATAACCT
GCCCAGGATCTCCCTCAGTTGCCAAGCTGAGACTTGTTGTATTTC
ACCAAAATGCAGCTGTGTTTCACAACCACCATTCTGTACTGTCTT
TTGTTCAATTTTTAGCTGTTTCCACTTTTCAGTATGATCGTTTCT
TTGAGAAAAAAATTGATGAGAAAAAGAATGACCACACCTATCGAG
TTTTTAAAACTGTGAACCGGCGAGCACACATCTTCCCCATGGCAG
ATGACTATTCAGACTCCCTCATCACCAAAAAGCAAGTGTCAGTCT
GGTGCAGTAATGACTACCTAGGAATGAGTCGC
homologous arm 2:
(SEQ ID NO: 9)
TGTGGGGCAGTTATGTAAGTAGCCCTTGGCTTTCAAATATTACTG
TTGTTATTTGGCAAGCCAATGATGATGTATAGGGGTTGGATCTTT
TATGGAGGGAACATTCAGTAGCTGAAAGTGTGCCATAGCAAAATA
CTATTCTTAGCTTCTGAAAAATATCTACAGATTACTTTTAAAGGA
ACTCTAATATGCAGGTAGCTGCTGGAGCCCCTTAACTTGTGAGGG
TTCAAGCTTATAGGCTCAATGACCACACACGTCAGTCCACTTTCT
ATCACTCTGCAAGTGAGTGTGCCTTTAACACCAGGCAGTCTTTCA
CAGAGACATCCTTGGTTGTGGCTGTGAGTGGAGAAATACGAATCT
AGCAGCGCTAGAGAAAAAGCTACTCCAAAAGACATGTTAAAAAGG
ATGGTAGATCCCATCACTCGTCCTTCAGGAGGCTCACCATCCTAG
GGATGCTGGAGAGAGTTGCTGGCCAGATTCTCCAAAGGCTTGCTA
AAGAGGGTCCCGGTTTGAGGTTTAATGATAGTGATATATCCAAAC
ATTACATGACTGGCTTGCAGAGGGTAACCACCATCCAGGATGTCC
CTCTAGAGTTTGCTTTTTCTTAAGCTAACATGTTATTGGAAAGAA
TAATGTTTCTCAGAAAATAAACCTTAGTTTCTTAGAAAAGAAACT
CAGCTAATACCAAGCACTTACTGACTGTTAAATGAACACTTGTTC
TTGATGATTCCTGGAGGTATCATCACCCTAGCATTGACACCTTCT
CCCACCTAGTCTGAAGATGAAATCAGTTG

The following guide sequence was cloned into the SmartNuclease vector (pSN): GATGGCACACAGCT TCCGTC (SEQ ID NO:10). The resultant pHR and pSN plasmids were co-transfected into A549 cells by Lipofectamine. Cells were selected by hygromycin. Western blot for ALAS1 protein was performed to identify individual clones that had endogenous ALAS1 knocked out.

Next, full-length human ALAS2 was re-expressed in these cells via lentiviral transduction (pL VX-EF1a-human-ALAS2-IRES-geneticin). After geneticin selection, cells were transduced with a lentivirus to introduce a shRNA against human FECH with a puromycin marker (shRNA sequence: GACCATATTGAAACGCTGTAT (SEQ ID NO:11)). The resultant cells have endogenous ALAS1 knocked out, ALAS2 overexpressed, and FECH knocked down. These cells will be referred as A549-ALAS2 cells.

For PPIX cell assays, on day 1, 10,000 cells/100 ul RPMI (10% FBS) were seeded in each well of a 96-well plate. On day 3, medium was replaced with fresh RPMI (10% FBS) supplemented with 2.5 mM glycine and different concentrations of compounds. On day 4, PPIX fluorescence, used as a surrogate for PPIX level, was measured using a fluorescence plate reader (ex 410 nm and em 690 nm). Cell viability was then determined via CellTiter-Glo assay (Promega). Data were background subtracted and normalized to values from DMSO-treated cells, which was arbitrarily set as 100%.

### In Vitro XLP Cell Model

On day 1, hindlegs from ALAS2^{delAT/Y} mice were harvested. The tips of the bones were cut. Bone marrow cells were flushed out using FBS, strained through a 70-um cell strainer, and spun down at 300 g for 5 mins. Lineage positive cells were depleted using the Direct Lineage Cell Depletion kit (mouse) from Miltenylbiotec. Cell pellets were resuspended in 1 ml MACS buffer supplemented with 50 ul/ml rat serum. Samples were transferred to a 14 ml round-bottom tube and 50 uL/ml of isolation cocktail was added each samples and were then incubated on ice for 15 mins, followed by addition of 75 ul of vortexed RapidSpheres. After a 10-min incubation on ice, sample volume was brought up to 5 ml with MAC buffer. Cells were magnetically separated and re-suspended in differentiation medium (IMDM medium supplemented with 200 ug/ml holo-transferrin, 10 ug/ml insulin, 2 mM glutamine, 0.1 mM BME, 100 ng/ml IGF1, 10 uM dexamethasone, 80 ng/ml mouse EPO, 10 ng/ml mouse SCF, 15% v/v FBS, and 1% v/v detoxified albumin) at 0.5 million cells/ml. 100 ul of cells were plated into one well of a type-1 collagen-coated 96-well plate.

On day 2, differentiation medium was removed. Cells were then treated with 100 ul of EDM medium (IMDM medium with 20% FBS, 2 mM glutamine, and 0.1 mM BME) containing different concentrations of compounds for 48 hours, at which point PPIX fluorescence was measured and cell viability determined via Cell-TiterGlo.

36.6. Treating XLP Mice with Compound E1.

ALAS2^{delAT/Y} mice were randomized based on weight and blood PPIX fluorescence into two groups. Group 1 (n=15) was dosed with Compound E1 suspended in saline at 10mg/ml at 12mg/kg BID via oral gavage 8 hours apart. 0.5, 6, and 24 hours after first dose, mice (n=5 per time point) were euthanized by cardiac puncture. Group 2 (ALAS2^{delAT/Y}, n=4) and Group 3 (WT C₅₇/B6, n=5) were mocked treated with vehicle as controls. Plasma was collected by centrifugation of whole blood and used for measuring of plasma exposure of Compound El. Briefly, 40 μl of plasma was mixed with 200 ul of methanol containing 4-nitrobenzaldehyde (10 mg/mL). The derivatization was carried out at room temperature for 1 hour. The mixture was then centrifugated at 5800 rpm for 10 min. An aliquot of 100 μl was mixed with 10 μl of acetonitrile:water (1:1, v/v) containing Oslamid (2 ug/mL, IS). The sample was subsequently mixed and 1 μl of the supernatant was injected into the UPLC-MS/MS system. The LC separation was carried out using a Thermo Hypersil Gold (2.1×50 mm, 1.9 um) set at 40° C., and a gradient starting at 80% water (with 0.1% formic acid) and 20% methanol (with 0.1% formic acid) that reached 95% methanol (with 0.1% formic acid) in 2.5 min with a flow rate of 0.4 ml/min. The MRM transition, 246 to 98, was monitored for Compound E1. Calibration standards and quality control samples were prepared in blank mouse plasma. The standard curve had a coefficient of determination (R²) value>0.98 in a linear regression. The quality control samples had a precision and accuracy within +20% of theoretical values. The peak area ratios of analyte relative to the internal standard (IS) were used for Compound E1 quantitation. To collect bone marrow tissues, both femurs and tibias were harvested. Muscles and all connective tissues were removed. With both ends of the bones snipped off, each femur/tibia set (2 sets per mouse) was placed in a 2 ml Eppendorf tube stacked end-to-end and spun at 4° C. for 10 mins at ×13,000 g. Bones were then carefully removed. The bone marrow was used to measure three different endpoints: Compound E1, 5-aminolevulinic acid (5-ALA) and protoporphyrin IX (PPIX). The bone marrow tissues pellets were homogenized with 4 volume of PBS (pH_{7.4}) to allow the parallel measurements of our different endpoints. A 40 μl aliquot of bone marrow homogenate was then derivatized using the same method described for plasma. Subsequently, 1 μl of the derivatized bone marrow extract was analyzed using the same UPLC-MS/MS method than previously described to quantify Compound E1. A second aliquot of bone marrow homogenate was further diluted in in 3 volume of PBS (v/v) to achieve and 20 fold dilution factor. 80 μl of the 20 fold diluted bone marrow extract was combine with 80 μl of a label internal standard ¹³C_{5, 15}N-5-aminlevulinic acid prepared in PBS. Subsequently the 160 μl solution was loaded on an Oasis MCX solid phase extraction. Using standard procedure recommended by the manufacture, the 5-ALA and the label IS was extracted. Then the extract was further derivatized using 200 μl of 3N HCl in N-butanol for 1 hour at room temperature. Following the derivatized step, the sample was dried using nitrogen gas. The final sample was reconstituted in 150 μl of 20% methanol in water and 5 μl was injected into the UPLC/MS-MS system. The LC separation was carried out using a Thermo Hypersil Gold AQ (3×100 mm, 3 μm) set at 40° C., and a gradient starting at 80% water (with 0.1% formic acid) and 20% methanol (with 0.1% formic acid) that reached 95% methanol (with 0.1% formic acid) in 2.5 min with a flow rate of 0.4 ml/min. The MRM transition, 188.1 to 114, was monitored for to quantified derivatized 5-ALA. The last aliquot of bone marrow homogenate used to measure PPIX was extracted using a liquid:liquid extraction. The extracting solution was composed of acetonitrile:methanol:formic acid:water (40:40:40:10:10, v/v/v/v) and 4 volumes were added to the bone marrow homogenate to obtain a diluted-homogenized bone marrow sample. Subsequently the sample was vortex and centrifuged at 14,000 rpm for 5 minutes. 10 μl of the diluted-homogenized bone marrow was further mixed with 10 μl the previous extract solution to which an additional 200 μl of acetonitrile:methanol:formic acid (50:50:5, v/v/v) containing 5 ng/ml candesartan ciloexetil (IS) was added. The sample was mixed well and centrifuged at 5800 rpm, 4 C for 10 min. A final step dilution was carried out by combining 50 μl of the supernatant with 50 μl of 0.1% formic acid in methanol:water (1:1. v/v), and 5 μl was injected into the UPLC/MS-MS system. The LC separation was carried out using a Thermo Hypersil Gold C8 (2.1×50 mm, 1.9 um) set at 40° C., and a gradient starting at 50% mobile phase A (0.2% formic acid, methanol:water, 1:1, v/v) and 50% mobile phase B (0.2% formic acid, acetonitrile:methanol:water, 40:50:10, v/v/v) that reached 95% mobile phase B in 2 min with a flow rate of 0.6 ml/min. The MRM transition, 563.2 to 431.2, was monitored for to quantified derivatized PPIX.

### Example 37

### Expression and Purification

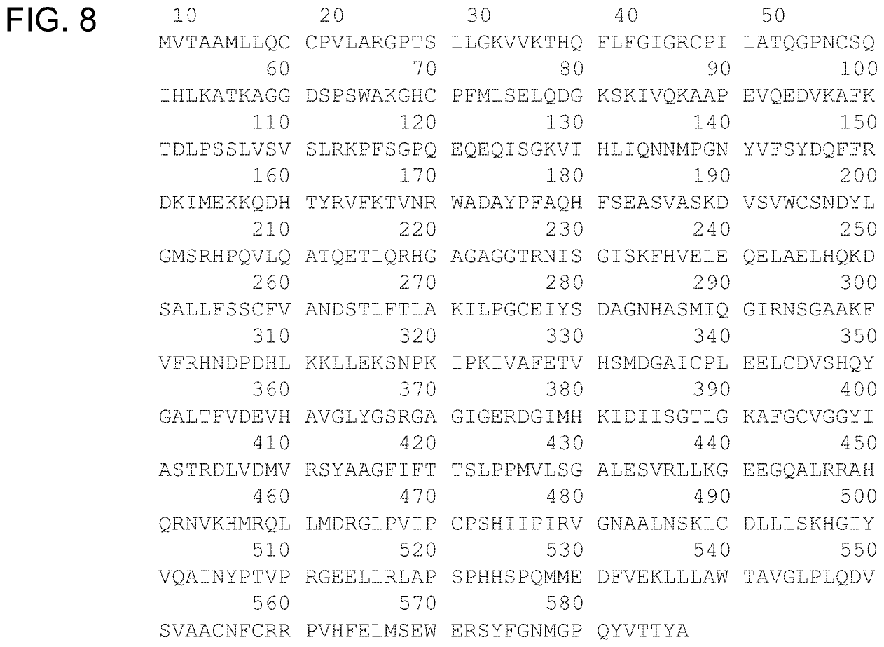

ALAS2 sequence (see FIG. 8) (residues 79-587) was expressed in E. coli as a TEV cleavable C-terminally MBP tagged construct. Purification steps included an amylose column, followed by MBP tag cleavage and passage through a Ni-NTA column to remove cleaved MBP. ALAS2 was further purified and exchanged into storage buffer (50 mM Tris, 500 mM NaCl, 1 mM DTT, 10 uM PLP, pH 8.0) by size exclusion through a Superdex 200 column and stored at −80° C.

### Example 38

### General IC₅₀ Assay Protocol

Preparation of stock solutions: All solutions were prepared by standard methods with the exception of glycine and succinyl CoA. A 1 M solution of glycine was prepared and the pH adjusted to 8.0 by addition of 1 N HCl. A 1 mM solution of succinyl CoA was prepared in 20 mM phosphoric acid, pH 2.0 to ensure its stability to hydrolysis.

Routine compound testing for IC₅₀ measurements: Assays were carried out in 96-well V-bottom polypropylene plates (Greiner-Bio cat. no. 651201) at room temperature. Test compound dilution series and DMSO blanks (1 μL) were dispensed into the 96-well plates. A solution of 1.1× assay mix was prepared to contain, in the final 1× assay conditions: 5 μg/mL ALAS2 (for the version 1 assay) or 2 ug/mL (for the version 2 assay), 20 mM sodium phosphate pH 8.0, 3.5 mM glycine, 1 mM MgCl₂, 30 mM NaCl, 0.03% BSA, 1 mM EDTA. Assay mix (45 μL) was added to each well, and compound and assay mix were pre-incubated for 1 h at room temperature. Assays were then initiated by addition of 5 μL 1 mM succinyl CoA to each well to give a final concentration of 100 μM. Reactions proceeded for 1 h (version 1 assay) or 2 h (version 2 assay) at room temperature, then were quenched by addition of 200 μL acetonitrile containing 1% formic acid and 0.2 μg/mL ¹³C, ¹⁵N aminolevulinic acid (ALA) as an internal standard. The plates were centrifuged at 4° C.for 1 min at 3500 rpm, then 200 μL of supernatant was transferred to a fresh 96-well plate for detection of ALA by high throughput mass spectrometry.

Measurement of ALA concentration by high throughput mass spectrometry: ALA was monitored in positive mode on a Sciex QTRAP 6500 connected to a Shimadzu 20AD LC using the conditions outlined in the table below. Samples were transferred from the assay plate and loaded onto a ZIC HILIC Trap column (Optimize Technologies, 4 μL, 10 μM) at a flow rate of 1.2 mL/min in 99% acetonitrile, 1% ddH₂O containing 0.5% formic acid to remove nonvolatile components with a 6 s desalt cycle. Analytes were eluted with 95% acetonitrile, 5% ddH₂O containing 0.5% formic acid in 12 s elution cycles. Concentrations of ALA in each sample were measured by comparison to an authentic standard curve, and IC₅₀s were determined by fitting to the log(compound) vs. response equation Y=bottom+(top-bottom)/(1+10\hat{ }((logIC₅₀-X)*HillSlope)).

TABLE 3
Mass spectrometry settings for analysis of ALA.

| | Q1 Mass | Q3 Mass | Time | DP | CE |
|---|---|---|---|---|---|
| ID | (m/z) | (m/z) | (ms) | (V) | (V) |
| ALA_1 | 132.0 | 114.0 | 100 | 10 | 10 |
| ALA_2 | 132.0 | 86.0 | 100 | 10 | 20 |
| 13C, 15N | 138.0 | 120.0 | 100 | 10 | 11 |
| ALA_1 | | | | | |
| 13C, 15N | 138.0 | 91.0 | 100 | 10 | 20 |
| ALA_2 | | | | | |

While a number of embodiments have been described, the scope of this disclosure is to be defined by the appended claims, and not by the specific embodiments that have been represented by way of example. The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1-35. (canceled)

36. A mouse (Mus musculus) as an animal model for X-linked protoporphyria (XLP), wherein the mouse comprises a genomic ALAS2 (5-AminoLevulinic Acid Synthase 2)-delAT mutation at the mouse ALAS2 locus, wherein the genomic ALAS2-delAT mutation encodes a mutant ALAS2 protein with a C-terminal deletion, and corresponds to or recapitulates the human ALAS2-delAT (c. 1699_1670AAT) mutation in an XLP human patient.

37. The mouse of claim 36, which has an elevated level of blood protoporphyrin IX (PPIX) and Zn-PPIX compared to a syngeneic wild-type mouse.

38. The mouse of claim 36, which is a C₅₇BL/6 mouse.

39. The mouse of claim 36, which is a male (ALAS2^{delAT/Y}).

40. The mouse of 38 claim 36, which is a female.

41. The mouse of claim 40, which is homozygous for the genomic ALAS2-delAT mutation (ALAS2^{delAT/delAT}).

42. The mouse of claim 40, which is heterozygous for the genomic ALAS2-delAT mutation.

43. The mouse of claim 36, which comprises the genomic DNA sequence of SEQ ID NO: 5.

44. The mouse of claim 36, which is generated by CRISPR/Cas9-mediated homology-directed repair (HDR) that deletes the AT dinucleotide in the genomic ALAS2-delAT mutation.

45. The mouse of claim 44, wherein the CRISPR/Cas9-mediated HDR utilizes a single guide RNA (sgRNA) comprising the nucleotide sequence of SEQ ID NO: 4.

46. The mouse of claim 44, wherein the CRISPR/Cas9-mediated HDR utilizes a donor DNA having the polynucleotide sequence of SEQ ID NO: 5.

47. The mouse of claim 44, wherein the CRISPR/Cas9-mediated HDR is carried out by microinjecting into the pronucleus of a mouse zygote an mRNA encoding Cas9, an sgRNA, and a single-stranded DNA (ssDNA).

48. The mouse of claim 36, wherein the mouse further comprises (i) at least one expression cassette comprising a polynucleotide encoding a reverse tetracycline-controlled transactivator (rtTA) under transcriptional control of a Rosa26 promoter and (ii) at least one expression cassette comprising a polynucleotide encoding an ALAS2 shRNA sequence (shALAS2) under transcriptional control of a TRE promoter.

49. The mouse of claim 48, wherein the expression cassette comprising a polynucleotide encoding an rtTA is inserted into chromosome 6.

50. The mouse of claim 48, wherein the expression cassette comprising a polynucleotide encoding shALAS2 is inserted into chromosome 11 at the ColAl locus.

51. The mouse of claim 48, wherein the mouse is heterozygous for the ALAS2-delAT mutation, heterozygous for rtTA (rtTA+/−) and heterozygous for shALAS2 (rtTA+/−).

52. The mouse of claim 48, wherein the shALAS2 comprises the sequence UGAAAAAUUGGUCAUAACCGAA (SEQ ID NO: 6).

53. A mouse comprising (i) at least one expression cassette comprising a polynucleotide encoding a reverse tetracycline-controlled transactivator (rtTA) under transcriptional control of a Rosa26 promoter and (ii) at least one expression cassette comprising a polynucleotide encoding an ALAS2 shRNA (shALAS2) under transcriptional control of a TRE promoter.

54. The mouse of claim 53, wherein the expression cassette comprising a polynucleotide encoding an rtTA is inserted into chromosome 6.

55. The mouse of claim 53, wherein the expression cassette comprising the polynucleotide encoding shALAS2 is inserted into chromosome 11 at the ColAl locus.

56. The mouse of claim 53, wherein the mouse is heterozygous for rtTA (rtTA+/−) or homozygous for rtTA (rtTA+/+).

57. The mouse of claim 53, wherein the mouse is heterozygous for shALAS2 (shALAS2|/−) or homozygous for shALAS2 (shALAS2|/|).

58. The mouse of claim 53, wherein the shALAS2 comprises the sequence UGAAAAAUUGGUCAUAACCGAA (SEQ ID NO: 6).
